(19) United States
(12) Patent Application Publication
(10) Pub. No.: US 2022/0175766 A1
(43) Pub. Date: Jun. 9, 2022

(54) COMPOSITIONS AND METHODS OF TREATMENT

(71) Applicant: GB004, Inc., San Diego, CA (US)

(72) Inventors: Barrett Levesque, San Diego, CA (US); Derek Moe, San Diego, CA (US); Nathaniel Santos Martinez, San Diego, CA (US)

(21) Appl. No.: 17/379,855

(22) Filed: Jul. 19, 2021

Related U.S. Application Data
(63) Continuation of application No. 17/115,655, filed on Dec. 8, 2020, now abandoned.
(60) Provisional application No. 63/065,374, filed on Aug. 13, 2020.
(60) Provisional application No. 62/971,824, filed on Feb. 7, 2020.
(60) Provisional application No. 62/945,753, filed on Dec. 9, 2019.

(51) Int. Cl. A61K 31/496; A61K 9/20

(52) U.S. Cl. CPC A61K 31/496; A61K 9/2095; A61K 9/2045; A61K 9/2013

(57) ABSTRACT

Pharmaceutical compositions are provided containing tert-butyl-{[1-(4-chlorobenzyl)-3-hydroxy-2-oxo-1,2-dihydropyridin-4-yl]methyl}piperazine-1-carboxylate or a pharmaceutically acceptable salt thereof, more particularly to certain orally deliverable immediate release solid pharmaceutical compositions containing a pharmaceutically acceptable salt of tert-butyl-{[1-(4-chlorobenzyl)-3-hydroxy-2-oxo-1,2-dihydropyridin-4-yl]methyl}piperazine-1-carboxylate and orally deliverable delayed release solid pharmaceutical compositions containing tert-butyl-{[1-(4-chlorobenzyl)-3-hydroxy-2-oxo-1,2-dihydropyridin-4-yl]methyl}piperazine-1-carboxylate or a pharmaceutically acceptable salt of. Also provided are methods and uses related to said compositions, as well as processes for the preparation of said compositions, and certain dosage regimens for the administration thereof.

This application claims the benefit of priority to U.S. Provisional Application Nos. 62/945,753, filed Dec. 9, 2019; 62/971,824, filed Feb. 7, 2020; and 63/065,374, filed Aug. 13, 2020, which applications are hereby incorporated by reference in their entireties.

### TECHNICAL FIELD

The present invention relates to pharmaceutical compositions containing tert-butyl-{[1-(4-chlorobenzyl)-3-hydroxy-2-oxo-1,2-dihydropyridin-4-yl]methyl}piperazine-1-carboxylate (hereinafter also referred to as the “Compound 1”) or a pharmaceutically acceptable salt thereof, and more particularly to certain orally deliverable solid pharmaceutical compositions containing Compound 1 or a pharmaceutically acceptable salt thereof; to the use of said compositions as a medicament; to processes for the preparation of said compositions; and to certain dosage regimens.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Inflammatory Bowel Disease (IBD) is characterized by chronic inflammation of the digestive tract. It is the result of an unrelenting, severe inflammatory response mounted against an environmental trigger in a genetically susceptible host. The inflammatory response causes a breach in intestinal barrier integrity which allows an influx of luminal antigens, establishing a cycle of inflammation and epithelial injury that perpetuates the disease process. Symptoms of IBD include severe, persistent diarrhea, rectal bleeding, abdominal pain, fatigue and weight loss. IBD disorders include Ulcerative Colitis (UC), which causes long-lasting inflammation and sores (ulcers) in the innermost lining of the large intestine (colon) and rectum; and Crohn's disease, which involves inflammation of the digestive tract lining, often spreading deep into the affected tissues. IBD is associated with a significantly reduced quality of life and increased mortality, especially among patients diagnosed at early ages.

The incidence and prevalence of IBD is highest in Europe and North America, with rates rising in other geographies. Thus, the already considerable socioeconomic burden of the disease will grow in the absence of more effective approaches to treatment and prevention.

Current management of IBD generally depends on the nature and severity of disease at presentation. For IBD not responding to 5-aminosalicylates (5-ASA), immunosuppression with corticosteroids, azathioprine and anti-tumor necrosis factor (TNF) antibodies has been the mainstay of treatment.

Despite the improved efficacy of IBD treatment with anti-TNF agents, a large subset of patients either do not respond adequately (˜30%) or do not achieve long-term remission (another ˜30%). Surgery is required for 20-30% of UC patients. In addition, side effects of immunosuppressive and anti-TNF agents, including opportunistic infection and malignancy, limit the risk-benefit equation. Moreover, the expense and inconvenience of administration of anti-TNF agents adds to the socioeconomic burden of the disease.

Newer immune-targeted therapies, especially those targeting gut selective leukocyte trafficking, have shown promising efficacy and acceptable safety profiles. Vedolizumab, the first of these gut selective agents, targets the activated form of α4β7 integrin on T cells blocking the gut-homing interaction with MAdCAM-1 (mucosal vascular addressing cell adhesion molecule 1). Clinical trial data suggest the efficacy of Vedolizumab is comparable to the anti-TNF agents though with lower rates of opportunistic infection. However, this still leaves many patients with IBD inadequately treated and dependent on the expense and inconvenience of a biological treatment.

While advances have been made in the treatment of IBD, there remains a significant need for new methods of treating this condition.

Prolyl-4-hydroxylase (PHD) inhibition has been shown to reduce disease severity in murine models of colitis (Robinson A., et al., Gastroenterology (2008), 145-155; Cummins E. P., et al., Gastroenterology (2008), 156-165). The proposed mechanism by which prolyl hydroxylase inhibitors exert their therapeutic activity is through hypoxia-inducible factor (HIF)-1α stabilization, which stimulates the augmentation and healing of the intestinal epithelial barrier (Keely S., et al., FASEB J. (2009), 1338-1346).

Compound 1 is a potent inhibitor of PHD. It is believed Compound 1 is selective for PHD-2 over PHD-1, resulting in specific stabilization of (HIF)-1a and reduced risk of off-target effects. Compound 1 is tert-butyl-{[1-(4-chlorobenzyl)-3-hydroxy-2-oxo-1,2-dihydropyridin-4-yl]methyl}piperazine-1-carboxylate and has the following structure:

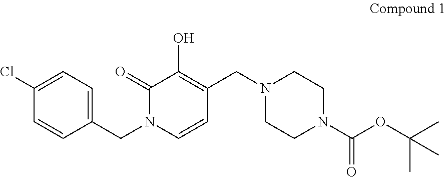 [SMILES: CC(C)(C)OC(=O)N1CCN(Cc2ccn(Cc3ccc(Cl)cc3)c(=O)c2O)CC1]

Compound 1 is disclosed in International Patent Application WO 2011/057121 (Example 1).

Whilst Compound 1 has been found to be particularly potent and effective in preclinical models of IBD, the unique physicochemical and biopharmaceutical properties of the molecule present particular challenges for development into a suitably acceptable dosage form.

Compound 1 can be classified as a BCS Class 2 compound (according to the Biopharmaceutical Classification System as defined by the “Guidance for Industry: Waiver of In Vivo Bioavailability and Bioequivalence studies for immediate release solid oral dosage forms based on a Biopharmaceutics Classification System”) which indicates it has a low solubility/dissolution rate and high permeability. Such compounds with low solubility can typically exhibit low and/or variable bioavailability and indeed the bioavailability of Compound 1 from a conventional solution formulation is poor.

Compound 1 is a weakly basic compound and has two basic groups with pKa's of 6.5 and 9.8. The pKa value expresses the strength of acids and base, i.e. the tendency for an acid to lose a proton or a base to accept a proton (Bronsted J. N. Rec. trav. Chim. (47), 718, 1923) and the protonation and deprotonation of the basic groups in the compound has a marked effect upon the solubility of Compound 1 in aqueous media. Consequently, the solubility of Compound 1 is highly dependent upon pH. For example, Compound 1 is soluble at pH 1 but is practically insoluble above pH 4, with limited solubility regained at very high pH values (i.e. above 10), due to the weakly acidic hydroxy moiety on the pyridone ring.

Compounds which have pH-dependent solubility, particularly basic compounds, may exhibit undesirable pharmacokinetic properties such as problems in their absorption, possibly producing low or variable bioavailability between patients and between doses.

A factor which can affect the absorption of an orally-administered drug is the changing pH experienced by the drug as it passes through the GI tract. Typically a drug may be absorbed in a number of different sites along the GI tract following oral administration for example, the cheek lining, stomach, duodenum, jejunum, ileum and colon. The pH may be different at each site of absorption with the pH significantly different from the stomach (pH 1 to 3.5) to the small intestine and the colon (pH 4-8). When the solubility of a drug varies with pH the drug may precipitate from solution as it passes through the GI tract. This can result in variability in the extent and/or rate of absorption between doses and between patients, since the drug needs to be in solution to be absorbed.

Although Compound 1 has a reasonably high solubility in the acidic environment of the stomach, it is not significantly absorbed from this area. The site of highest intrinsic absorption for Compound 1 is thought to be the upper intestine. However, in this region of the GI tract the pH is relatively high compared to that in the stomach and Compound 1 has a reduced solubility at this higher pH. As a result Compound 1 is prone to precipitate from solution as it passes from the acidic environment of the stomach to the higher pH environment of the upper GI tract (such as the upper intestine), resulting in reduced and/or variable absorption of Compound 1.

Furthermore, in view of the particular pH sensitivity of Compound 1, even small variations in local pH may have a significant effect upon its pharmacokinetic profile. The pH of the GI tract can also vary as a result of, for example, whether a patient is in a fed or fasted state and the rate of gastric emptying. The combination of the pH-sensitive solubility profile of Compound 1, together with the variability of the pH in the GI tract, may result in a high degree of inter-patient variability in the bioavailability and/or plasma concentrations of Compound 1, which may lead to possible sub-optimal treatment efficacy in a proportion of patients. There is therefore a need for approaches to improve the pharmacokinetic properties of Compound 1.

The Applicants have identified certain salt forms of Compound 1 with improved solubility and these new forms offer certain benefits. However, in order to formulate a pharmaceutically active compound, such as a salt, into a suitably acceptable dosage form, the active compound should, in addition to possessing acceptable solubility properties, also suitably possess other properties, such as acceptable stability and handling properties. In this respect, particular problems occur with salt forms of Compound 1.

The Applicant has found that a hydrochloride salt of Compound 1 (1:1 drug:HCl of Compound 1) is particularly liable to dissociate into its free-base form during processing and/or storage of formulations comprising the salt. Such conversion is not only undesirable because the free-base form has poorer biopharmaceutical properties (solubility and dissolution rate) but also has consequences for the chemical stability of the compound. Unusually, Compound 1 contains a tert-butyloxycarbonyl (Boc) group which is liable to chemical degradation in the presence of an acidic environment. Therefore, conversion of the hydrochloride salt of Compound 1 to the free-base form should be avoided as it would not only be expected to cause a reduction in bioavailability and/or lead to an increase in inter- and intra-patient variability in plasma concentrations, both of which could lead to less than optimal treatment for patients, but it could even result in hydrolysis of the Boc group and chemical degradation of Compound 1.

To confound things further, the said hydrochloride salt form has been found by the Applicants to possess poor powder flow and compression properties, making it very difficult to formulate as a solid dosage form.

There is, therefore, a need for new pharmaceutical compositions containing Compound 1 or a pharmaceutically acceptable salt thereof, particularly compositions in which the stability of a salt form can be maintained during processing and storage to provide acceptable absorption and/or bioavailability of Compound 1 upon dosing. Such compositions also need to possess suitable stability, handling and processing properties to enable large-scale dosage manufacture. This is a particularly challenging mix of problems to address and the Applicant has found that various attempts to formulate the hydrochloride salt of Compound 1, e.g. as a direct compression tablet dosage form, have proved challenging with issues such as inferior flow, poor processability and chemical stability and poor disintegration in pH 6.8 buffer leading to agglomeration and poor dispersion and dissolution of Compound 1 upon dosing.

### SUMMARY OF INVENTION

Applicants have surprisingly found that stability of a salt form of Compound 1 can be maintained in granule-based compositions of the present invention. Certain granule-based compositions of the present invention display improved disintegration and dissolution properties leading to improved absorption and/or bioavailability upon dosing. Certain compositions also offer improved chemical stability, powder flow and compression properties.

In addition to the above, the Applicants have also found that certain oral delayed release compositions comprising Compound 1 or a pharmaceutically acceptable salt thereof provide benefits in respect to targeted release and biopharmaceutical performance. Surprisingly, certain delayed release compositions achieve colonic tissue concentration levels that are comparable to oral immediate release solution and immediate release tablet compositions without the need for any appreciable systemic exposure. Such delayed release compositions offer a number of advantages in respect to the targeted treatment of IBD.

In a first aspect, the present invention provides an immediate release (IR) solid pharmaceutical composition for oral administration comprising a granulate, wherein the granulate comprises a pharmaceutically acceptable salt of Compound 1:

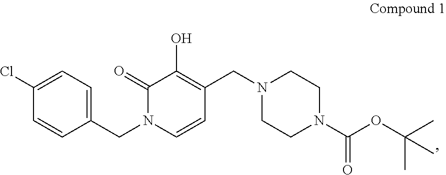 [SMILES: CC(C)(C)OC(=O)N1CCN(Cc2ccn(Cc3ccc(Cl)cc3)c(=O)c2O)CC1]

and one or more pharmaceutically acceptable excipients.

In a second aspect, the present invention provides a process for forming an immediate release solid pharmaceutical composition according to the first aspect, the process comprising the steps of:
a) mixing a pharmaceutically acceptable salt of Compound 1 with one or more acceptable excipients selected from a filler, a diluent, a binder and a disintegrant;
b) adding to the mixture from step a) a wetting agent, water and optionally a binder;
c) subjecting the mixture from step b) to wet granulation;
d) drying the wet granules from step c); and
e) milling the dried granules to a particle size diameter of <1 mm.

In a third aspect, the present invention provides a delayed-release (DR) solid pharmaceutical composition for oral administration comprising Compound 1, or a pharmaceutically acceptable salt thereof:

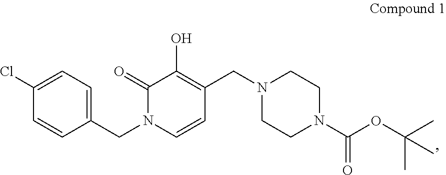 [SMILES: CC(C)(C)OC(=O)N1CCN(Cc2ccn(Cc3ccc(Cl)cc3)c(=O)c2O)CC1]

and one or more pharmaceutically acceptable excipients.

In a fourth aspect, the present invention provides a process for forming a delayed-release solid pharmaceutical composition according to the third aspect.

In a fifth aspect, the present invention provides a method for treating a disease or condition mediated alone or in part by PHD, such as inflammatory bowel disease, by administration to a subject in need thereof an immediate release (IR) solid pharmaceutical composition according to the first aspect, or a delayed-release (DR) solid pharmaceutical composition according to the third aspect, and in a related aspect provides such compositions for use in the treatment of diseases or conditions mediated alone or in part by PHD, such as inflammatory bowel disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graphical plot of Compound 1 solubility (log₁₀ ng/mL) vs pH at 22° C., as described in Example 1

FIG. 2 is a Flow diagram for a representative Compound 1⋅HCl Direct Compression Manufacturing Procedure

FIG. 3 is a Flow diagram for a representative Compound 1⋅HCl High Shear Granulation Manufacturing Procedure

FIG. 4 is a Flow diagram for the manufacturing process for Compound 1⋅HCl IR Tablets as described in Example 17

FIG. 5 is a Flow diagram for the manufacturing process for Compound 1⋅HCl DR Tablets as described in Example 18

FIG. 6 is a Comparison of dissolution rates for Compound 1⋅HCl from 60 mg IR and DR Tablets, as described in Example 19

FIG. 7 is a Flow diagram for the Compound 1⋅HCl High Shear Granulation Manufacturing Procedure used for Formulations F25-F28

FIG. 8 is a graphical plot of Compound 1 mean plasma concentration, over time 0-16 hr for the clinical trial described in example 23 (all subjects)

FIG. 9 is a graphical plot of Compound 1 mean plasma concentration, over time 0-16 hr for the clinical trial described in example 23 (without vomiting subjects)

FIG. 10 is a graphical representation of the Study Scheme for the clinical trial described in example 24

FIG. 11 is a graphical plot of Compound 1 mean plasma concentration, over time 0-25 hr for the clinical trial described in example 24, on day 1

FIG. 12 is a graphical plot of Compound 1 mean plasma concentration, over time 0-25 hr for the clinical trial described in example 24, on day 7

FIG. 13 shows the box plot of the proportions of HIF-1a positive cells evaluated at baseline and Day 8.

FIG. 14 is a graphical representation of the Study Scheme for the clinical trial described in example 25

FIG. 15 is a is a graphical representation of the Study Scheme for the clinical trial described in Example 26

FIG. 16 is a graphical plot showing Compound 1 mean plasma concentrations following single (day 1) and multiple (day 7) dosing of: (top) 120 mg solution, 120 mg immediate release tablet and 120 mg & 240 mg delayed release tablets in fasted subjects; and (bottom) 120 mg immediate release tablet in fasted subjects and 240 mg immediate release tablet in fed subjects; over time 0-24 hr for the clinical trial described in Example 26.

FIG. 17 is a graphical plot of median colon tissue concentrations (rectum—left; sigmoid—right) for the dosing cohorts in the clinical trial described in Example 26.

FIG. 18 is a plot of Compound 1 total colon concentrations for the dosing cohorts in the clinical trial described in Example 26.

### DETAILED DESCRIPTION

### Definitions

‘Immediate Release’ or ‘IR’ as used in the present application in its conventional sense refers to a dosage form that provides for release of a compound immediately after administration. For example, an immediate release formulation means a formulation in which the dissolution rate of the drug from the formulation is 75% or more after 30 minutes from the beginning a dissolution test, which is carried out in accordance with a dissolution test (paddle method) described in the United States Pharmacopoeia (USP) under the conditions that 900 mL of an appropriate test fluid (such as 0.01N hydrochloric acid) is used and the paddle rotation speed is 75 rpm. Conveniently, ‘Immediate Release’ or ‘IR’ as used in the present application refers to a formulation or composition which rapidly disintegrates and disperses to release a drug compound after oral administration to a subject. More conveniently, it refers to a formulation or composition for which the dissolution rate of the drug from the formulation is 75% or more after 30 minutes (such as after 15 minutes) from the beginning a dissolution test, which is carried out in accordance with a USP 2 dissolution test (paddle method) under the conditions that 900 mL of 0.01 N hydrochloric acid is used, the temperature is 37° C. and the paddle rotation speed is 75 rpm. In terms of in vivo pharmacokinetics, immediate release refers to a formulation or composition which typically provides systemic (plasma) levels of active pharmaceutical agent shortly after oral dosing. More conveniently, it refers to a formulation or composition which provides a geometric mean maximum plasma concentration (Cₘₐₓ) of the compound after oral dosing of at least 5 ng/ml, such as at least 10 ng/ml within at least 120 minutes, conveniently within at least 60 minutes or 30 minutes.

‘Delayed Release’ or ‘DR’ as used in the present application refers to a dosage form that provides for release of a compound after administration at a slower rate than that from an immediate release formulation, or release of the active compound starts at a later point in time compared with an immediate release composition (such as at 30 min or more later such as, e.g., 1 hour or more later or 2 hours or more later or 3 hours or more later than an immediate release composition). Conveniently, ‘Delayed Release’ or ‘DR’ as used in the present application refers to a formulation or composition which, after oral administration to a subject, does not undergo disintegration and dissolution in the acidic environment of the stomach. In terms of in vitro dissolution testing, delayed release refers to a formulation or composition which typically undergoes less than 5% dissolution in acidic aqueous media. More conveniently, it refers to a formulation or composition in which the dissolution rate of the drug from the formulation is 5% or less (such as 2% or less) after 30 minutes from the beginning a dissolution test, which is carried out in accordance with a USP 2 dissolution test (paddle method) under the conditions that 900 mL of 0.01 N hydrochloric acid is used, the temperature is 37° C. and the paddle rotation speed is 75 rpm.

‘Granulate’ as used in the present application refers to a granular material, which is the result of the agglomeration of fine particles into larger granules. The granules may be packaged, for example, in a capsule for administration; dosed as a suspension or slurry in a liquid; dosed in a sachet; formed into pellets or beads by processes known in the art; or they may be formed into a tablet (e.g. a caplet), mini-tablet, micro-tablet or pill by compression or other processes known in the art. Granules may typically be sized in the range 0.1 to 2.0 mm. Conveniently, the granules may have an average particle size in the range 0.2 to 0.5 mm. Conveniently, the granules are formed into a mini-tablet or micro-tablet. Micro-tablets may typically be sized in the range 1 to 4 mm.

The term ‘pharmaceutically acceptable excipient’ is used herein to refer to an essentially pharmacologically inert, non-toxic substance, e.g. that has been approved for inclusion in pharmaceutical products. Examples of excipients classes includes fillers and diluents, binders, disintegrants, surfactants, wetting agents, lubricants, preservatives, colorants, flavouring agents, sweeteners and coatings.

As used herein, the term ‘intimate association’ refers to at least two components which are intimately mixed together. Compound 1 has a propensity to self-agglomerate in aqueous media such as pH 6.8 buffer. It has been found that co-processing of the Compound 1 or a pharmaceutically acceptable salt thereof and one or more excipients (such as a wetting agent) results in these components being intimately associated with one another leading to improved dispersion, and it is postulated that surface-to-surface interactions between particles of the Compound 1 or a pharmaceutically acceptable salt thereof are reduced in the intimate mixture such that dispersal is improved on exposure to aqueous media.

### Compound 1

As mentioned above, the present invention relates to orally deliverable solid pharmaceutical compositions containing Compound 1 or a pharmaceutically acceptable salt thereof, to the use of the compositions as a medicament; to processes for the preparation of said compositions; and to certain dosage regimens.

Compound 1, as referred to in the present invention, is tert-butyl 4-((1-(4-chlorobenzyl)-3-hydroxy-2-oxo-1,2-dihydropyridin-4-yl)methyl)piperazine-1-carboxylate, which has the following structure:

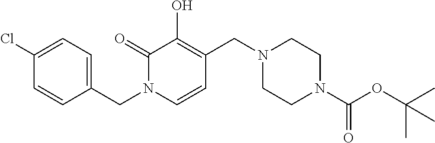 [SMILES: CC(C)(C)OC(=O)N1CCN(Cc2ccn(Cc3ccc(Cl)cc3)c(=O)c2O)CC1]

As depicted above, Compound 1 is shown as a “free base”. In some embodiments, a pharmaceutically acceptable salt of Compound 1 is used. Conveniently, the pharmaceutically acceptable salt of Compound 1 is a HCl salt of Compound 1 (in a 1:1 molar ratio). The HCl salt of Compound 1 is depicted below:

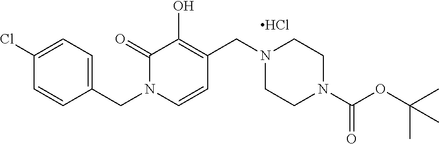 [SMILES: CC(C)(C)OC(=O)N1CCN(Cc2ccn(Cc3ccc(Cl)cc3)c(=O)c2O)CC1 ; Cl]

The molecular weight of Compound 1 is 433.93. The molecular weight of the HCl salt of Compound 1 is 470.39. 1.0 g of Compound 1-HCl salt contains 0.92 g Compound 1 free base. 1.08 g Compound 1-HCl salt contains 1.0 g Compound 1 free base:

| | | | |
|---|---|---|---|
| Compound 1-HCl | MW = 470.39 | 1.0 g | 1.08 g |
| Compound 1 | MW = 433.93 | 0.92 g | 1.0 g |
| HCl | MW = 36.46 | 0.08 g | 0.08 g |

Throughout the specification, unless specified otherwise, references to the amount of Compound 1 will be understood to refer to the amount of the parent compound (free base equivalent), even if the compound is present as a salt of Compound 1. Purely by way of example, reference to 120 mg of Compound 1 or a salt thereof, will be understood to refer to 120 mg of the free base, or a salt of Compound 1 with 120 mg of free base equivalent; in the context of the anhydrous mono-hydrochloride salt of Compound 1, 130 mg of the salt provides 120 mg of Compound 1 (free base equivalent).

Compound 1 is disclosed in U.S. Pat. Nos. 8,536,181 and 8,999,971 (incorporated herein in their entirety). Compound 1 is a prolyl-hydroxylase (PHD) inhibitor. Inhibition of PHD enzymes stabilizes hypoxia inducible factor (HIF) transcription. HIFs are a family of transcription factors that modulates the body's response to stress. Compound 1 selectively stabilizes HIF-1a, which plays a key protective role in the intestinal wall. In preclinical models of IBD, Compound 1 promotes both resolution of intestinal inflammation and restoration of intestinal epithelial barrier function. Compound 1 is being developed as a treatment of IBD.

The following references provide details about PHD inhibitors: (a) Marks et al, “Regulation of IL-12p40 by HIF controls Th1/Th17 responses to prevent mucosal inflammation”, Mucosal Immunol. 2017, 10(5), 1224-1236; (b) Feinman et al, “Inhibition of HIF Prolyl Hydroxylases Mitigate Gut Graft-Versus-Host Disease”, Blood 2016, 128(22), 3349; (c) Marks et al, “Oral delivery of prolyl hydroxylase inhibitor: AKB-4924 promotes localized mucosal healing in a mouse model of colitis”, Inflamm Bowel Dis. 2015, 21(2), 267-75; (d) Keely et al, “Contribution of epithelial innate immunity to systemic protection afforded by prolyl hydroxylase inhibition in murine colitis”, Mucosal Immunol. 2014, 7(1), 114-23; (e) Campbell et al, “Transmigrating Neutrophils Shape the Mucosal Microenvironment through Localized Oxygen Depletion to Influence Resolution of Inflammation”, Immunity 2014, 40(1), 66-77; and (f) Okumura et al, “A New Pharmacological Agent (AKB-4924) Stabilizes Hypoxia Inducible Factor (HIF) and Increases Skin Innate Defenses Against Bacterial Infection”, J Mol Med (Berl), 2012, 90(9), 1079-89.

### Immediate Release (IR) Solid Compositions

In a first aspect, the present invention provides an immediate release solid pharmaceutical composition for oral administration comprising a granulate, wherein the granulate comprises a pharmaceutically acceptable salt of Compound 1:

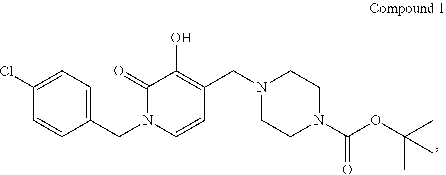 [SMILES: CC(C)(C)OC(=O)N1CCN(Cc2ccn(Cc3ccc(Cl)cc3)c(=O)c2O)CC1]

and one or more pharmaceutically acceptable excipients.

The Applicants have surprisingly found that stability of a salt form of Compound 1 can be maintained in certain granule-based compositions of the present invention and dissociation into the free-base form and/or chemical degradation of the compound can be avoided or reduced. Certain granule-based compositions of the present invention display improved disintegration and dissolution properties leading to good absorption and/or bioavailability upon dosing. Certain immediate release solid compositions according to the present invention have been found to give comparable systemic exposure of Compound 1 after 7 days of oral dosing as observed with an equivalent dose formulated as a HP-βCD solution (see Example 27). Furthermore, certain immediate release compositions have been shown to deliver higher colonic tissue levels of Compound 1 than were achieved via oral solution dosing (see Example 28). Certain compositions also possess improved chemical stability, powder flow and compression properties.

In the first aspect of the present invention a pharmaceutically acceptable salt of Compound 1 is used. Suitable pharmaceutically acceptable salts include acid-addition salts of the basic piperazine nitrogen in Compound 1 and also metal salts of the weakly acidic hydroxyl group in Compound 1.

Acid-addition salts include salts with inorganic or organic acids. Inorganic acid salts include hydrochloric, hydrobromic, sulfuric and phosphoric acid salts. Organic acid salts include trifluoroacetic, acetic, formic, citric, maleic, succinic, lactic, glycolic, tartaric, methanesulfonic and p-toluenesulfonic acid salts. Conveniently, the pharmaceutically acceptable salt is an inorganic acid salt. More conveniently, the pharmaceutically acceptable salt is a hydrochloric acid salt.

Metal salts include alkali metal salts and alkali earth metal salts, such as sodium, potassium, calcium or magnesium salts. Conveniently, the pharmaceutically acceptable salt is a calcium salt.

In an embodiment, the pharmaceutically acceptable salt is a hydrochloride salt. In an embodiment, the pharmaceutically acceptable salt is a hydrochloride salt and the ratio of Compound 1 to HCl is about 1:1.

In an embodiment, the hydrochloride salt is a hydrate. In a convenient embodiment, the hydrochloride salt is a monohydrate. In a convenient embodiment, the hydrochloride salt is a crystalline monohydrate, which is characterized by X-Ray (Cu K radiation in transmission mode using 40 kV/40 mA generator settings) diffraction peaks at 15.1, 17.4, 19.8, 20.0 and 20.6±0.2 degrees 29 (Form A). In a convenient embodiment, the hydrochloride salt is a crystalline monohydrate, which is characterized by a melting point of 191-194° C.

In an embodiment, the hydrochloride salt is anhydrous. In a convenient embodiment, the hydrochloride salt is a crystalline anhydrous compound, which is characterized by X-Ray (Cu K radiation in transmission mode using 40 kV/40 mA generator settings) diffraction peaks at 9.0, 15.2, 16.8, 18.6 and 20.3±0.2 degrees 29 (Form B). In a convenient embodiment, the hydrochloride salt is a crystalline anhydrous compound, which is characterized by a melting point of 195-198° C.

In an embodiment, the granulate comprises a mixture of the hydrochloride salt as a crystalline monohydrate and the hydrochloride salt as an amorphous compound. Analysis of the physical form of Compound 1 in the granulate may be carried out by techniques known to the person of skill in the art, e.g. XRPD, FTIR, Raman, or solid-state NMR. In a particular embodiment, the granulate comprises a mixture of the Compound 1 hydrochloride salt as a crystalline monohydrate and the Compound 1 hydrochloride salt as an amorphous compound, wherein the amount of amorphous compound present is less than 10% by weight of the total amount of Compound 1 hydrochloride salt present in the granulate. Conveniently, the amount of amorphous compound present is less than 9% (such as less than 8%, less than 7%, less than 6%, less than 5%, less than 4%, less than 3%, less than 2%, or less than 1%) by weight of the total amount of Compound 1 hydrochloride salt present in the granulate.

In an alternative embodiment, the granulate comprises a mixture of the hydrochloride salt as a crystalline monohydrate (Form A) and the hydrochloride salt as a crystalline anhydrous (Form B) compound.

In an embodiment, the one or more pharmaceutically acceptable excipients comprise a wetting agent. It is postulated that when a wetting agent is intimately mixed with a pharmaceutically acceptable salt of Compound 1 within a granulate composition, the disintegration of the composition and dispersal of Compound 1 or a pharmaceutically acceptable salt thereof in aqueous media is promoted. In an embodiment, the pharmaceutically acceptable salt of Compound 1 is in intimate association with the wetting agent in the granulate. In an embodiment, the pharmaceutically acceptable salt of Compound 1 is in intimate association with the wetting agent and one or more additional pharmaceutically acceptable excipients (optionally selected from disintegrant, binder and diluent).

The wetting agent is typically a surfactant or an emulsifier. In a convenient embodiment, the wetting agent is a non-ionic surfactant. Non-ionic surfactants can be characterized according to their hydrophilic-lipophilic balance (HLB). HLB values are commonly used to define emulsifiers and/or surfactants and refer to the hydrophilic-lipophilic balance of the given compound. HLB values can be calculated according to the methods of Griffin [Griffin, J. Soc. Cosmetic Chem. (1949), 311-326; Griffin, J. Soc. Cosmetic Chem. (1954), 249-256] as follows:

HLB=20×(MW-H/MW-T)

wherein MW-H is the molecular weight of the hydrophilic portion of the compound and MW-T is the molecular weight of the total compound. For example, for the emulsifier PEG100 stearate, MW-H is the molecular weight of the ethylene glycol portions of the molecule which is 100×44 (MW ethylene oxide monomer=44 g/mol)=4400. Stearic acid has a molecular weight of 284.5 g/mol, so MW-T=4684.5. Therefore, the HLB value for PEG100 stearate is calculated at 18.8. PEG-80 sorbitan monooleate (sold as Tween® 80 or Polysorbate 80) has a HLB value of 15. HLB values for a selection of emulsifiers and commercially available excipients are listed in the table below.

| Material | Trade name | HLB |
|---|---|---|
| Glycerol monooleate | Peceol ® | 3.5 |
| PEG-6 glyceryl oleate | Labrafil ® M1944CS | 4 |
| Glycerol monolinoleate | Maisine ® 35-1 | 4 |
| Diethylene glycol monoethyl ether | Transcutol ®, Carbitol ® | 4.2 |
| Sorbitan monooleate | Span ® 80 | 4.3 |
| Triglycerol diisostearate | Plurol ® diisostearique | 4-5 |
| Propylene glycol monocaprylate | Capmul ® PG8 | 5-6 |
| Medium chain monoglycerides | Akoline ® MCM | 5-6 |
| Glyceryl mono-/dicaprylate | Capmul ® MCM | 5.5-6 |
| Polyglyceryl-3 dioleate | Plurol ® oleique CC497 | 6 |
| Propylene glycol monocaprylate | Capryol ® 90 | 6 |
| Polyglyceryl-6 distearate | Plurol ® stearic WL1009 | 9-10 |
| PEG-35 castor oil | Cremophor ® EL, | 12-14 |
| | Kolliphor ® EL | |
| D-alpha-tocopheryl PEG-1000 | Vitamin E TPGS | 13 |
| succinate | | |
| Caprylocaproyl PEG-8 glycerides | Labrasol ® | 14 |
| PEG-32 glyceryl laureate | Gelucire ® 44/14 | 14 |
| PEG-40 hydrogenated castor oil | Cremophor ® RH40, | 14-16 |
| | Kolliphor ® RH40 | |
| PEG-80 sorbitan monooleate | Tween ® 80 | 15 |
| PEG-20 sorbitan monolaurate | Tween ® 20 | 16.7 |
| PEG-100 stearate | Myrj ™ S100 | 18.8 |
| Poloxamer 407 | Pluracare ® F127 | 21.5 |
| Poloxamer 188 | Pluracare ® F68 | 29 |

Ionic surfactants generally have higher HLB values than non-ionic surfactants. Sodium lauryl sulfate (SLS), for example, has a HLB value of 40.

In an embodiment, the wetting agent is generally regarded as safe for oral administration to humans.

In an embodiment, the wetting agent has a hydrophilic-lipophilic balance (HLB) between 5 and 25. Conveniently, the wetting agent has a hydrophilic-lipophilic balance (HLB) between 8 and 20. More conveniently, the wetting agent has a hydrophilic-lipophilic balance (HLB) between 12 and 18, such as between 13 and 18, between 13 and 17, or about 14 to 16.

In an embodiment the wetting agent is water soluble. In an embodiment, the wetting agent has an aqueous solubility greater than 10 g/litre, such as greater than 10 g/litre, greater than 20 g/litre, greater than 50 g/litre, or greater than 75 g/litre.

In an embodiment, the wetting agent is water soluble and has a hydrophilic-lipophilic balance (HLB) between 12 and 18 In an embodiment, the wetting agent has an aqueous solubility greater than 10 g/litre and a hydrophilic-lipophilic balance (HLB) between 12 and 18. In an embodiment, the wetting agent has an aqueous solubility greater than 10 g/litre and a hydrophilic-lipophilic balance (HLB) between 13 and 18, such as between 13 and 17, or between 14 and 16. In an embodiment, the wetting agent has an aqueous solubility greater than 50 g/litre and a hydrophilic-lipophilic balance (HLB) between 13 and 18, such as between 13 and 17, or between 14 and 16.

In an embodiment the wetting agent is a non-ionic wetting agent selected from polyol esters, polyoxyethylene esters and poloxamers. In an embodiment, the polyol esters are selected from one or more of glycol esters, glycerol esters and sorbitan derivatives. In a convenient embodiment, sorbitan derivates comprise polysorbate esters (such as polysorbate 20, polysorbate 40, polysorbate 60 and polysorbate 80). Most conveniently the wetting agent is PEG-80 sorbitan monooleate (polysorbate 80).

In an embodiment, the wetting agent is present in the composition at about 0.1 to 5.0% w/w of the total weight of the composition. In an embodiment, the wetting agent is present in the composition at about 0.5 to 2.5% w/w. In a convenient embodiment, the wetting agent is present in the composition at about 0.5 to 1.5% w/w, such as about 0.75 to 1.25% w/w, or about 1.0% w/w.

It has been found that acidic environments are capable of cleaving the Boc group of Compound 1. It may therefore be advantageous to avoid incorporating overly acidic excipients in the compositions of the present invention, or carefully selecting grades of excipients such that their acidity is within acceptable ranges. It may be desirable to avoid excipients, such as buffering agents, capable of generating acidic micro-environments (e.g. pH less than 3) within the granulate. In a convenient embodiment, the one or more pharmaceutically acceptable excipients are not acidic. In a convenient embodiment, the one or more pharmaceutically acceptable excipients have a pH of greater than 3.5 when dissolved or slurried in water. In a more convenient embodiment, the one or more pharmaceutically acceptable excipients have a pH of greater than 5 (conveniently greater than 6) when dissolved or slurried in water. In yet a more convenient embodiment, the one or more pharmaceutically acceptable excipients have a pH of greater than 6.5 when dissolved or slurried in water. In an embodiment, the one or more pharmaceutically acceptable excipients have a pH of greater than 6 (such as greater than 6.5), when a 5 g sample of the excipient is dissolved or slurried with 40 ml of water for 20 minutes, centrifuged and the pH of the solution or supernatant is measured.

For example, the excipient microcrystalline cellulose (MCC) is a purified, partially depolymerised cellulose prepared by treating cellulose pulp with mineral acids. As such, the pH of different batches of MCC may vary between approximately 5.0 and 7.5 (when a 5 g sample of MCC is slurried with 40 ml of water for 20 minutes, centrifuged and the pH of the supernatant is measured).

Typically, after formation of the granulate compositions they are dried and milled. It may be desirable to have a low water content. In an embodiment, the composition has a moisture level of less than 6% w/w, such as less than 5% w/w or less than 4% w/w. Conveniently, the composition has a moisture level of less than 3% w/w.

In an embodiment, the one or more pharmaceutically acceptable excipients comprise a binder. A binder helps to hold the granulate mixture together. In an embodiment, the binder is a cellulose ether-based binder (such as hydroxypropyl cellulose or methyl cellulose). In a convenient embodiment, the binder is selected from hydroxy propyl cellulose, hypromellose, povidone, starch, methylcellulose, gelatin, pregelatinized starch, and xanthan gum. In a more convenient embodiment, the binder is hydroxy propyl cellulose.

In an embodiment, the one or more pharmaceutically acceptable excipients comprise a disintegrant. A disintegrant increases water wicking into the granulate core and therefore facilitates disintegration of the solid composition. In an embodiment, the disintegrant is selected from crospovidone, croscarmellose, sodium starch glycolate and low substituted hydroxypropyl cellulose. In a more convenient embodiment, the disintegrant is crospovidone.

In an embodiment, the one or more pharmaceutically acceptable excipients comprise a diluent. In an embodiment, the diluent is selected from lactose, pregelatinized starch, microcrystalline cellulose and silicified microcrystalline cellulose.

In an embodiment, the one or more pharmaceutically acceptable excipients comprise microcrystalline cellulose and lactose within the granulate. Conveniently, the w/w ratio of microcrystalline cellulose:lactose within the granulate is between 1:1 and 3:1 (such as about 2:1).

In an embodiment, the diluent is selected from lactose and pregelatinized starch, or mixtures of the two. In an embodiment, the diluent is selected from lactose monohydrate and pregelatinized starch, or mixtures of the two. Conveniently, the one or more pharmaceutically acceptable excipients comprise lactose monohydrate and pregelatinized starch within the granulate.

In an embodiment, the immediate release composition according to the present invention comprises about 10-40% w/w of the pharmaceutically acceptable salt of Compound 1. Conveniently, the composition comprises 15-30% w/w of the pharmaceutically acceptable salt of Compound 1. In one embodiment, the composition comprises 15-20% (such as 16-18%) w/w of the pharmaceutically acceptable salt of Compound 1. In another embodiment, the composition comprises 25-30% (such as 26-28%) w/w of the pharmaceutically acceptable salt of Compound 1.

In an embodiment, the immediate release composition according to the present invention comprises 20 to 150 mg (such as 50 to 70 mg, about 60 mg, 110 to 130 mg, or about 120 mg) of the pharmaceutically acceptable salt of Compound 1. In an embodiment, the immediate release composition according to the present invention comprises 120 to 360 mg (such as 180 to 300 mg, 200 to 280 mg, 220 to 260 mg, or about 240 mg) of the pharmaceutically acceptable salt of Compound 1. In an embodiment, the immediate release composition according to the present invention comprises 20 to 360 mg (such as about 60 mg, about 120 mg, or about 240 mg) of the hydrochloride salt of Compound 1.

In an embodiment, the one or more pharmaceutically acceptable excipients do not comprise a buffering agent. Buffering agents are weak acids or bases which are capable of buffering the pH micro-environment the composition is exposed to in vivo after oral dosing. Examples of buffering agents include citric acid, lactic acid, tartaric acid.

The granulate is composed of granules, which may typically be sized in the range 0.1 to 2.0 mm. Conveniently, the granules within a granulate have an average particle size in the range 0.2 to 0.5 mm. In an embodiment, the granulate comprises granules having an average particle size between 200 and 500 m, such as between 250 and 400 μm.

In an embodiment, the granulate comprises granules having a particle size distribution of 0-10% less than between 74 μm; 10-20% between 74 and 125 μm; 10-20% between 125 and 177 μm; 30-50% between 177 and 420 μm; 5-30% between 420 and 595 μm; and 0-25% between 595 and 841 μm.

In an embodiment, the granulate comprises granules having a particle size distribution of 30-50% of granules having particle sizes between 177 and 420 μm; and/or less than 20% of granules having particle sizes between 125 and 177 μm. In an embodiment, the granulate comprises granules having a particle size distribution of 30-40% of granules having particle sizes between 177 and 420 μm; and/or 10-20% of granules having particle sizes between 125 and 177 μm.

In contrast to fine powder formulations used in direct compression tabletting, the granulate compositions according to the present invention typically have higher bulk densities, and lower Carr's Index and Hausner ratio values. These properties are indicative that the granulates have superior powder flow and compression properties.

In an embodiment, the composition has one or more of the following properties:
a bulk density of >0.45 g/ml;
a Carr's Index of <35%; and
a Hausner Ratio of <1.5.

In an embodiment, the composition has one or more of the following properties:
a bulk density of >0.5 g/ml;
a Carr's Index of <30%; and
a Hausner Ratio of <1.35.

In an embodiment, the composition has one or more of the following properties:
a bulk density of >0.5 g/ml;
a Carr's Index of <25%; and
a Hausner Ratio of <1.3.

In an embodiment, the composition has one or more of the following properties:
a bulk density of >0.55 g/ml;
a Carr's Index of <20%; and
a Hausner Ratio of <1.25.

The immediate release compositions according to the present invention undergo rapid disintegration and dispersal in aqueous media (such as 0.01N hydrochloric acid or pH 6.8 buffer).

In an embodiment the dissolution rate of the drug from the immediate release composition is 75% or more after 30 minutes from the beginning a dissolution test, which is carried out in accordance with a dissolution test (paddle method) described in the United States Pharmacopoeia (USP) under the conditions that 900 mL of 0.01 N hydrochloric acid is used at 37° C. and the paddle rotation speed is 75 rpm. Conveniently, the dissolution rate of the drug from the immediate release composition is 80% or more after 15 minutes from the beginning a dissolution test, which is carried out in accordance with a dissolution test (paddle method) described in the United States Pharmacopoeia (USP) under the conditions that 900 mL of 0.01N hydrochloric acid is used at 37° C. and the paddle rotation speed is 75 rpm.

In an embodiment, the immediate release composition undergoes substantially complete disintegration and dispersal in 0.01N hydrochloric acid in less than 5 minutes at 37° C. using USP disintegration apparatus. In an embodiment, the immediate release composition undergoes substantially complete disintegration and dispersal in pH 6.8 aqueous media in less than 5 minutes at 37° C. using USP disintegration apparatus. In an embodiment, the immediate release composition undergoes substantially complete disintegration and dispersal in pH 1-7 aqueous media in less than 5 minutes at 37° C. using USP disintegration apparatus.

The immediate release compositions according to the present invention provide systemic (plasma) levels of Compound 1 shortly after oral dosing. In an embodiment, an immediate release composition according to the present invention provides a geometric mean maximum plasma concentration (Cₘₐₓ) of Compound 1 after oral dosing of at least 5 ng/ml, such as at least 10 ng/ml. In an embodiment, an immediate release composition according to the present invention provides a geometric mean maximum plasma concentration (Cₘₐₓ) of Compound 1 of at least 5 ng/ml, such as at least 10 ng/ml, after oral dosing of at least 120 mg of a pharmaceutically acceptable salt of Compound 1. In an embodiment, an immediate release composition according to the present invention provides a geometric mean maximum plasma concentration (Cₘₐₓ) of Compound 1 of at least 5 ng/ml, such as at least 10 ng/ml, after oral dosing of at least 120 mg of a pharmaceutically acceptable salt of Compound 1 to a fasted subject. In an embodiment, an immediate release composition according to the present invention provides a geometric mean maximum plasma concentration (Cₘₐₓ) of Compound 1 of at least 10 ng/ml, after oral dosing of at least 240 mg of a pharmaceutically acceptable salt of Compound 1 to a fed subject. In an embodiment, an immediate release composition according to the present invention provides a geometric mean maximum plasma concentration (Cₘₐₓ) of Compound 1 of 5-25 ng/ml, such as 10-20 ng/ml, after oral dosing of 120-240 mg of a pharmaceutically acceptable salt of Compound 1 to a subject.

The immediate release compositions according to the present invention advantageously provide good colon tissue exposure of Compound 1 after oral dosing. Colon tissue concentrations can be measured by biopsy as described in the Examples section. In an embodiment, an immediate release composition as described herein provides colonic tissue exposure greater than or equal to the systemic exposure of Compound 1 following oral solution dosing of the composition to a subject. Advantageously, the immediate release solid compositions as described herein deliver higher median levels of Compound 1 to the colon after oral dosing, than achieved with a corresponding dose formulated as an oral solution. In an embodiment, an immediate release composition as described herein provides median sigmoid colon tissue concentrations of greater than 50 ng/g (such as greater than 75 ng/g) after oral dosing of at least 120 mg of a pharmaceutically acceptable salt of Compound 1 to a fasted subject. In an embodiment, an immediate release composition as described herein provides median rectum colon tissue concentrations of greater than 25 ng/g (such as greater than 35 ng/g) after oral dosing of the composition comprising at least 120 mg of a pharmaceutically acceptable salt of Compound 1 to a fasted subject. In an embodiment, an immediate release composition as described herein provides median sigmoid colon tissue concentrations of greater than 100 ng/g (such as greater than 200 ng/g) after oral dosing of the composition comprising at least 240 mg of a pharmaceutically acceptable salt of Compound 1 to a fed subject. In an embodiment, an immediate release composition as described herein provides median rectum colon tissue concentrations of greater than 50 ng/g (such as greater than 75 ng/g) after oral dosing of the composition comprising at least 240 mg of a pharmaceutically acceptable salt of Compound 1 to a fed subject.

Colonic biopsies may also allow for measurement of the proportion of hypoxia-inducible factor (HIF)-1α positive cells in the biopsy samples both before (baseline) and after (e.g. at day 7) treatment with an immediate release solid composition as described herein. In an embodiment, an immediate release composition as described herein provides a greater than 10% (such as greater than 20%) increase from baseline in the proportion of HIF-1a positive cells in the colon after oral dosing of the composition to a subject. In an embodiment, an immediate release composition as described herein provides a greater than 10% (such as greater than 20%) increase from baseline in the proportion of HIF-1a positive cells in the colon after oral dosing of the composition comprising at least 240 mg of Compound 1 or a pharmaceutically acceptable salt thereof to a subject. In an embodiment, an immediate release composition as described herein provides a greater than 10% (such as greater than 20%) increase from baseline in the proportion of HIF-1α positive cells in the sigmoid colon tissue after oral dosing of the composition comprising at least 240 mg of Compound 1 or a pharmaceutically acceptable salt thereof to a subject.

The chemical stability of the pharmaceutically acceptable salt of Compound 1 in certain solid compositions of the present invention has been found to be very good even after several months under accelerated storage conditions. In an embodiment, the immediate release composition provides less than 5% chemical degradation of Compound 1 by HPLC when stored at 40° C. and 75% RH for 3 months. In an embodiment, the immediate release composition provides less than 5% chemical degradation of Compound 1 by HPLC when stored at 25° C. and 60% RH for 6 months. In an embodiment, the immediate release composition provides less than 2% chemical degradation of Compound 1 by HPLC when stored at 25° C. and 60% RH for 3 months.

Conveniently, the immediate release solid pharmaceutical composition as described herein may be formulated as a tablet. The term ‘tablet’ comprises tablets of any size suitable for oral administration, including micro-tablets and mini-tablets (which would typically be in the size range 1 to 4 mm). The composition may be encapsulated within a capsule, wherein the capsule is non-functional (i.e. it is readily soluble in aqueous media, so that the immediate release properties of the composition are not significantly affected). In an embodiment, the immediate release composition is formulated as a tablet and the tablet has an average hardness of greater than 7 kp, such as greater than 7.5 kp, greater than 8 kp, greater than 8.5 kp or greater than 9 kp. In an embodiment, the immediate release composition is formulated as a tablet and the tablet has an average hardness of 7-12 kp, such as 7.5-11 kp, 8-11 kp, or 8.5-10 kp.

When the composition is formulated as a tablet, prior to compression additional extra-granular excipients are typically mixed with the granules to improve the tablet processability and properties. Therefore, in an embodiment, the tablet comprises a granulate component and an extra-granulate component. In an embodiment, the granulate component comprises a pharmaceutically acceptable salt of Compound 1 and one or more pharmaceutically acceptable excipients selected from a filler, a diluent, a binder and a disintegrant. In an embodiment, the extra-granulate component comprises one or more pharmaceutically acceptable excipients selected from a filler, a diluent, a disintegrant and a lubricant. Suitable excipients for the extra-granulate component will be readily chosen by one of skill in the art and suitable fillers/diluents and disintegrants are described above. Lubricants may be added to solid compositions to reduce friction and sticking during tablet processing. Suitable lubricants comprise magnesium stearate, calcium stearate, hydrogenated vegetable oil, stearic acid, sodium stearyl fumarate, mineral oil and polyethylene glycol. In an embodiment, the extra-granulate component comprises magnesium stearate. In an embodiment the composition comprises 0.25-1.5% w/w of lubricant (such as magnesium stearate).

In an embodiment, the immediate release solid pharmaceutical composition comprises a granulate, wherein the granulate comprises:
20-40% w/w of a pharmaceutically acceptable salt of Compound 1;
40-70% w/w of one or more diluents;
1-10% w/w of disintegrant;
1-10% w/w of binder; and
0.5-2.0% w/w of wetting agent.

In an embodiment, the immediate release solid pharmaceutical composition comprises a granulate, wherein the granulate comprises:
20-40% w/w of a pharmaceutically acceptable salt of Compound 1;
45-65% w/w of one or more diluents;
3-8% w/w of disintegrant;
3-8% w/w of binder; and
1-2% w/w of wetting agent.

In an embodiment, the immediate release solid pharmaceutical composition comprises a granulate, wherein the granulate comprises:
20-30% w/w of a pharmaceutically acceptable salt of Compound 1;
55-65% w/w of one or more diluents;
3-8% w/w of disintegrant;
3-8% w/w of binder; and
1-2% w/w of wetting agent.

In an embodiment, the immediate release solid pharmaceutical composition comprises a granulate, wherein the granulate comprises:
30-40% w/w of a pharmaceutically acceptable salt of Compound 1;
45-55% w/w of one or more diluents;
3-8% w/w of disintegrant;
3-8% w/w of binder; and
1-2% w/w of wetting agent.

In an embodiment, the immediate release solid pharmaceutical composition is formulated as a tablet and the tablet comprises a granulate component and an extra-granulate component, wherein the tablet comprises in the granulate component:
10-30% w/w of a pharmaceutically acceptable salt of Compound 1;
30-60% w/w of one or more diluents;
1-7% w/w of disintegrant;
1-7% w/w of binder; and
0.5-2.0% w/w of wetting agent;
   and the tablet comprises in the extra-granulate component:
20-30% w/w of one or more diluents;
1-5% w/w of disintegrant; and
0.25-1.5% w/w of lubricant.

In an embodiment, the immediate release solid pharmaceutical composition is formulated as a tablet and the tablet comprises a granulate component and an extra-granulate component, wherein the tablet comprises in the granulate component:
15-30% w/w of a pharmaceutically acceptable salt of Compound 1;
30-50% w/w of one or more diluents;
3-5% w/w of disintegrant;
3-5% w/w of binder; and
0.5-1.5% w/w of wetting agent;
   and the tablet comprises in the extra-granulate component:
20-30% w/w of one or more diluents;
2-4% w/w of disintegrant; and
0.5-1.5% w/w of lubricant.

In an embodiment, the immediate release solid pharmaceutical composition is formulated as a tablet and the tablet comprises a granulate component and an extra-granulate component, wherein the tablet comprises in the granulate component:
15-20% w/w of a pharmaceutically acceptable salt of Compound 1;
40-50% w/w of one or more diluents;
3-6% w/w of disintegrant;
3-6% w/w of binder; and
0.5-1.5% w/w of wetting agent;
   and the tablet comprises in the extra-granulate component:
22-27% w/w of one or more diluents;
2-4% w/w of disintegrant; and
0.5-1.5% w/w of lubricant.

In an embodiment, the immediate release solid pharmaceutical composition is formulated as a tablet and the tablet comprises a granulate component and an extra-granulate component, wherein the tablet comprises in the granulate component:
25-30% w/w of a pharmaceutically acceptable salt of Compound 1;
30-40% w/w of one or more diluents;
2-4% w/w of disintegrant;
4-6% w/w of binder; and
0.5-1.5% w/w of wetting agent;
   and the tablet comprises in the extra-granulate component:
22-27% w/w of one or more diluents;
2-4% w/w of disintegrant; and
0.5-1.5% w/w of lubricant.

Conveniently, the immediate release composition is substantially encapsulated in a water-soluble coating. The coating may be a capsule encapsulating the granulate, or it may be a coating substantially encapsulating the composition formulated as a tablet. In an embodiment, the composition is formulated as a tablet and the tablet is substantially encapsulated in a water-soluble (pH-independent—e.g. soluble in aqueous media at pH 1-8) coating. To prevent light degradation of the tablets on storage, in an embodiment, the water-soluble coating is also a UV-resistant coating. In an embodiment the coating is PVA-based.

In an embodiment, the immediate release composition is prepared by a wet granulation method. In an embodiment, the immediate release composition is obtainable by wet granulation. Wet granulation can be carried out by any known wet granulation process, including high-shear wet granulation and fluid bed granulation (see for example, Remington: The Science and Practice of Pharmacy, Edition, 22nd Edition, 2012). Conveniently, the wet granulation is carried out with high shear mixing.

### Processes for Forming Immediate Release Solid Compositions

In a second aspect, the present invention provides a process for forming an immediate release solid pharmaceutical composition according to the first aspect, the process comprising the steps of:
a) mixing a pharmaceutically acceptable salt of Compound 1 with one or more pharmaceutically acceptable excipients selected from a filler, a diluent, a binder and a disintegrant;
b) adding to the mixture from step a) a wetting agent, water and optionally a binder;
c) subjecting the mixture from step b) to wet granulation;
d) drying the wet granules from step c); and
e) milling the dried granules to a particle size diameter of <1 mm.

In an embodiment, the pharmaceutically acceptable salt of Compound 1 is a hydrochloride salt. In an embodiment, the pharmaceutically acceptable salt is a hydrochloride salt and the ratio of Compound 1 to HCl is about 1:1. In an embodiment, the hydrochloride salt is a hydrate. In a convenient embodiment, the hydrochloride salt is a monohydrate. In an alternative embodiment, the hydrochloride salt is anhydrous.

In an embodiment, the filler or the diluent in step a) are selected from one or more of lactose, pregelatinized starch, microcrystalline cellulose and silicified microcrystalline cellulose.

In an embodiment, the filler or the diluent in step a) are lactose and microcrystalline cellulose. Conveniently, the w/w ratio of microcrystalline cellulose:lactose is between 1:1 and 3:1 (such as about 2:1).

In an embodiment, the filler or the diluent in step a) are selected from lactose and pregelatinized starch, or mixtures of the two. In an embodiment, the filler or the diluent are selected from lactose monohydrate and pregelatinized starch, or mixtures of the two. Conveniently, the one or more pharmaceutically acceptable excipients comprise lactose monohydrate and pregelatinized starch. Conveniently, the w/w ratio of pregelatinized starch:lactose monohydrate is between 1:4 and 1:8 (such as about 1:6).

In an embodiment, the binder in step a) is selected from one or more of hydroxy propyl cellulose, hypromellose, povidone, starch, methylcellulose, gelatin, pregelatinized starch, and xanthan gum. In a convenient embodiment, the binder is hydroxy propyl cellulose.

In an embodiment, the disintegrant in step a) is selected from one or more of crospovidone, croscarmellose, sodium starch glycolate and low substituted hydroxypropyl cellulose. In a convenient embodiment, the disintegrant is crospovidone.

In an embodiment, the mixing in step a) is carried out under high shear. In an embodiment, the mixing in step a) is carried out in a high shear granulator. Conveniently, the high shear mixing is carried out with a mixer (impeller) speed of 100-500 rpm and a chopper speed of 1000-3000 rpm.

In an embodiment, the wetting agent in step b) has a hydrophilic-lipophilic balance (HLB) between 5 and 25. Conveniently, the wetting agent has a hydrophilic-lipophilic balance (HLB) between 8 and 20. More conveniently, the wetting agent has a hydrophilic-lipophilic balance (HLB) between 12 and 18, such as about 14 to 16.

In an embodiment the wetting agent in step b) is a non-ionic wetting agent selected from polyol esters, polyoxyethylene esters and poloxamers. In an embodiment, the polyol esters are selected from one or more of glycol esters, glycerol esters and sorbitan derivatives. In a convenient embodiment, sorbitan derivates comprise polysorbate esters (such as polysorbate 20, polysorbate 40, polysorbate 60 and polysorbate 80). Most conveniently the wetting agent is PEG-80 sorbitan monooleate (polysorbate 80).

In an embodiment, the wetting agent in step b) is dissolved in the water. Optionally the aqueous mixture in step b) also comprises a binder. Conveniently, the binder in step b) is selected from one or more of hydroxy propyl cellulose, hypromellose, povidone, starch, methylcellulose, gelatin, pregelatinized starch, and xanthan gum, such as hydroxy propyl cellulose.

In an embodiment, the aqueous mixture from step b) is applied to the mixture from step a) by spraying. Conveniently, the mixing from step a) is continued during the step b) addition of the aqueous wetting agent mixture.

Wet granulation can be carried out by any known wet granulation process, including high-shear wet granulation and fluid bed granulation (see for example, Remington: The Science and Practice of Pharmacy, Edition, 22nd Edition, 2012). In an embodiment, the wet granulation in step c) is carried out under high shear. Conveniently, the high shear mixing in step c) is carried out with a mixer (impeller) speed of 25-500 rpm and a chopper speed of 1000-3000 rpm.

In an embodiment, the drying in step d) is carried out at 20-100° C., such as 40-80° C., or conveniently 50-70° C. More conveniently, the drying in step d) is carried out at about 60° C. In an embodiment, the drying in step d) is carried out in a fluid bed dryer.

In an embodiment, after the granules have been milled in step e), they are screened through a US 20 mesh (0.841 mm sieve opening). Therefore, in an embodiment, after step e) the granules have a particle size diameter of less than 841 μm.

In an embodiment, the process also comprises a step f) of:
f) blending the milled granules from step e) with one or more pharmaceutically acceptable excipients selected from a filler, a diluent, a disintegrant and a lubricant.

In step f) the milled granulate is mixed with extra-granular excipients (selected from one or more of a filler, a diluent, a disintegrant and a lubricant). In an embodiment, the filler or the diluent in step f) are selected from one or more of lactose, pregelatinized starch, microcrystalline cellulose and silicified microcrystalline cellulose. In an embodiment, the filler or the diluent in step f) is lactose. In an embodiment, the filler or the diluent in step f) is lactose monohydrate.

In an embodiment, the disintegrant in step f) is selected from one or more of crospovidone, croscarmellose, sodium starch glycolate and low substituted hydroxypropyl cellulose. In a convenient embodiment, the disintegrant is crospovidone.

In an embodiment, the lubricant in step f) is selected from one or more of magnesium stearate, calcium stearate, hydrogenated vegetable oil, mineral oil and polyethylene glycol. In an embodiment, the lubricant is magnesium stearate.

In an embodiment, the components in steps a) to b) of the above process are added in the relative amounts described above in paragraphs [00101] to [00104].

In an embodiment, the components in steps a) to f) of the above process are added in the relative amounts described above in paragraphs [00105] to [00108].

In an embodiment, when binder is added wet in step b) to the dry mixture from step
a) comprising binder, the w/w ratio of wet binder:dry binder used is between 1:9 and 1:1, conveniently about 1:4.

When the composition is formulated within a capsule, then the process according to the second aspect of the invention typically further comprises the step g) of:
g) substantially encapsulating the granules from step f) in a UV-resistant capsule.

In an embodiment, the capsule is water-soluble (pH-independent—e.g. soluble in aqueous media at pH 1-8). In an embodiment the capsule is HPC, HPMC or PVA-based.

Alternatively, when the composition is formulated as a tablet then the process according to the second aspect of the invention typically further comprises the following steps:
g) compressing the blended granule mixture from step f) into a tablet; and
h) optionally substantially encapsulating the tablet from step g) in a UV-resistant coating.

In an embodiment, the compression in step g) gives an average tablet hardness of greater than 7 kp, such as greater than 7.5 kp, greater than 8 kp, greater than 8.5 kp or greater than 9 kp. In an embodiment, the compression in step g) gives an average tablet hardness of 7-12 kp, such as 7.5-11 kp, 8-11 kp, or 8.5-10 kp.

In an embodiment, the coating applied in step h) is soluble in aqueous media independent of the pH of the media. In an embodiment, the coating is water-soluble at between pH 1 and 8. In an embodiment the coating is PVA-based. In an embodiment, the coating is step h) is applied to a weight gain of 2-6%, such as 3-5%, or about 4%.

In an embodiment, there is provided an immediate release solid pharmaceutical composition obtainable by, obtained by or directly obtained by a process according to the second aspect of the invention.

### Delayed Release Solid Compositions

In a third aspect of the present invention there is provided a delayed-release solid pharmaceutical composition for oral administration comprising Compound 1 or a pharmaceutically acceptable salt thereof:

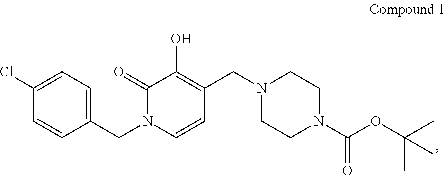 [SMILES: CC(C)(C)OC(=O)N1CCN(Cc2ccn(Cc3ccc(Cl)cc3)c(=O)c2O)CC1]

and one or more pharmaceutically acceptable excipients.

The oral delayed-release compositions comprising Compound 1 or a pharmaceutically acceptable salt thereof provide benefits in respect to targeted release of Compound 1 and biopharmaceutical performance. Surprisingly, certain delayed release solid compositions of the present invention have been found to achieve colonic tissue concentration levels of Compound 1, that are at least comparable to those achieved via oral dosing of a pharmaceutically acceptable salt of Compound 1 as either an immediate release solution, or an immediate release solid composition, without the need for any appreciable systemic exposure. Such compositions offer a number of advantages in respect to treatment of IBD, such as potentially minimising systemic side effects, e.g. those linked to elevated erythropoietin (EPO) and vascular endothelial growth factor (VEGF) levels.

The delayed-release solid pharmaceutical composition according to the present invention can be any composition suitable for oral administration, such as a tablet, a capsule, or granules or pellets, for example delivered in a sachet or capsule.

In the third aspect of the present invention, Compound 1 or a pharmaceutically acceptable salt thereof is used. In an embodiment, there is provided a delayed-release solid pharmaceutical composition for oral administration comprising Compound 1 (i.e. the free base) and one or more pharmaceutically acceptable excipients. In another embodiment, there is provided a delayed-release solid pharmaceutical composition for oral administration comprising a pharmaceutically acceptable salt of Compound 1 and one or more pharmaceutically acceptable excipients.

Suitable pharmaceutically acceptable salts include acid-addition salts of the basic piperazine nitrogen in Compound 1 and also metal salts of the weakly acidic hydroxyl group in Compound 1.

Acid-addition salts include salts with inorganic or organic acids. Inorganic acid salts include hydrochloric, hydrobromic, sulfuric and phosphoric acid salts. Organic acid salts include trifluoroacetic, acetic, formic, citric, maleic, succinic, lactic, glycolic, tartaric, methanesulfonic and p-toluenesulfonic acid salts. Conveniently, the pharmaceutically acceptable salt is an inorganic acid salt. More conveniently, the pharmaceutically acceptable salt is a hydrochloric acid salt.

Metal salts include alkali metal salts and alkali earth metal salts, such as sodium, potassium, calcium or magnesium salts. Conveniently, the pharmaceutically acceptable salt is a calcium salt.

In an embodiment, the pharmaceutically acceptable salt is a hydrochloride salt. In an embodiment, the pharmaceutically acceptable salt is a hydrochloride salt and the ratio of Compound 1 to HCl is about 1:1.

In an embodiment, the hydrochloride salt is a hydrate. In a convenient embodiment, the hydrochloride salt is a monohydrate. In a convenient embodiment, the hydrochloride salt is a crystalline monohydrate, which is characterized by X-Ray diffraction peaks at 15.1, 17.4, 19.8 and 20.0±0.2 degrees 2θ (Form A). In a convenient embodiment, the hydrochloride salt is a crystalline monohydrate, which is characterized by a melting point of 191-194° C.

In an embodiment, the hydrochloride salt is anhydrous. In a convenient embodiment, the hydrochloride salt is a crystalline anhydrous compound, which is characterized by X-Ray diffraction peaks at 9.0, 16.8 and 18.6±0.2 degrees 29 (Form B). In a convenient embodiment, the hydrochloride salt is a crystalline anhydrous compound, which is characterized by a melting point of 195-198° C.

In one embodiment, the delayed-release solid pharmaceutical composition is an erodible matrix comprising Compound 1 or a pharmaceutically acceptable salt thereof dispersed in the matrix. By erodible matrix is meant aqueous-erodible or water-swellable or aqueous soluble, in the sense of being either erodible or swellable or dissolvable in pure water or requiring the presence of an acid or base to ionize the polymeric matrix sufficiently to cause erosion or dissolution. When contacted with an aqueous environment, the erodible matrix imbibes water and forms an aqueous-swollen gel or “matrix” that Compound 1 or a pharmaceutically acceptable salt thereof can pass or diffuse through depending on its physicochemical properties. The aqueous-swollen matrix gradually erodes, swells, disintegrates or dissolves, thereby delaying the release of Compound 1 or a pharmaceutically acceptable salt thereof after oral administration. Suitable polymers for the erodible matrix are hydrogels such as synthetic polymers derived from vinyl, acrylate, methacrylate, urethane, ester and oxide monomers; or derivatives of naturally occurring polymers such as polysaccharides or proteins (including polysaccharides, gums, starches, alginates, collagen and cellulosics).

In another embodiment, the delayed-release solid pharmaceutical composition is a capsule comprising granules or pellets encapsulated within a delayed release capsule.

In an alternative embodiment, the delayed-release solid pharmaceutical composition is a readily-soluble capsule comprising granules or pellets, wherein the individual granules or pellets are coated with a delayed release coating.

In an alternative embodiment, the delayed-release solid pharmaceutical composition comprises a core and a delayed release coating substantially encapsulating the core. Conveniently, the delayed-release solid pharmaceutical composition is a tablet comprising a solid core and a delayed release coating substantially encapsulating the solid core. The term ‘tablet’ comprises tablets of any size suitable for oral administration, including micro-tablets and mini-tablets (which would typically be in the size range 1 to 4 mm).

In terms of the coating, ‘substantially’ means that the coating covers the majority of the surface of the core, such as greater than 75%, greater than 85%, or preferably greater than 95% of the surface of the core. In a convenient embodiment, the delayed release coating fully encapsulates the core.

In an embodiment, the delayed release coating dissolves at pH values greater than about 5.5. In an embodiment, the delayed release coating dissolves at pH values greater than about 6.0. In an embodiment, the delayed release coating dissolves at pH values greater than about 7.0. Conveniently, the delayed release coating dissolves at about pH 5.5.

In an embodiment, the delayed release coating comprises methyl acrylate-methacrylic acid copolymer, ethyl acrylate-methacrylic acid copolymer, hydroxy propyl methyl cellulose acetate succinate or cellulose acetate phthalate. In a convenient embodiment, the delayed release coating comprises methyl acrylate-methacrylic acid copolymer or ethyl acrylate-methacrylic acid copolymer. Conveniently, the delayed release coating is selected from Eudragit® L 100-55, Eudragit® FS30D, Eudragit® L100, Eudragit® L 12,5, Eudragit® L30 D-55, Eudragit® S100 and Eudragit® S12,5, such as Eudragit® L 100-55.

The thickness of the coating is important to ensure that the composition does not take up acid and disintegrate in the acidic environment of the stomach. In an embodiment, the delayed-release composition comprises a greater than 8% weight gain delayed release coating. In an embodiment, the delayed-release composition comprises a greater than 10% weight gain delayed release coating. In an embodiment, the delayed-release composition comprises about 12% weight gain delayed release coating. In an embodiment, the delayed-release composition comprises about 14% weight gain delayed release coating.

In an embodiment, the delayed-release composition further comprises an additional sub-coating beneath the delayed release coating. In an embodiment, the sub-coating is soluble in aqueous media independent of the pH of the media. In an embodiment, the sub-coating is water-soluble at between pH 1 and 8. In an embodiment the sub-coating is PVA-based. In an embodiment, the delayed-release composition comprises 2-6%, such as 3-5%, or about 4% weight gain of sub-coating.

Conveniently, the delayed-release composition comprises a core and the core comprises a granulate. The granulate may conveniently be a granulate as described above according to the first aspect of the invention. Therefore, all the embodiments described above for the first aspect (in relation to Compound 1 or a pharmaceutically acceptable salt thereof, the one or more pharmaceutically acceptable excipients, levels of the excipients, granule particle size distribution, bulk density, Carr's index and Hausner ratio of the composition, the extra-granulate component and tablet hardness) all apply equally to a delayed-release composition core comprising a granulate according to the third aspect of the invention.

In an embodiment, the delayed release composition according to the present invention comprises 20 to 150 mg (such as 50 to 70 mg, about 60 mg, 110 to 130 mg, or about 120 mg) of Compound 1 or a pharmaceutically acceptable salt thereof. In an embodiment, the delayed release composition according to the present invention comprises 120 to 360 mg (such as 180 to 300 mg, 200 to 280 mg, 220 to 260 mg, or about 240 mg) of Compound 1 or a pharmaceutically acceptable salt thereof. In an embodiment, the delayed release composition according to the present invention comprises 20 to 360 mg (such as about 60 mg, about 120 mg, or about 240 mg) of Compound 1 or the hydrochloride salt thereof.

In an alternative embodiment, the delayed-release composition is a multiparticulate composition comprising delayed release pellets or beads. The pellets may be formed from a granulate as described hereinabove, wherein the individual pellets are coated with a delayed release coating as described herein, or alternatively the pellets are encapsulated within a delayed release capsule. The delayed release capsule may conveniently have the same properties as the delayed release coatings described herein.

Alternatively, each pellet, or subunit, of the delayed release multiparticulate is an inert core coated with a drug layer comprising Compound 1 or a pharmaceutically acceptable salt thereof and the drug layer-coated core is then coated with a delayed release coating as described herein. In this embodiment, multiparticulates in the form of beads or pellets may be prepared by building the Compound 1 or a pharmaceutically acceptable salt thereof composition (drug plus optionally any excipients) up on a seed core by a drug-layering technique such as powder coating or by applying the Compound 1 or a pharmaceutically acceptable salt thereof composition by spraying a solution or dispersion of the Compound 1 or a pharmaceutically acceptable salt thereof in an appropriate solution/dispersion vehicle (e.g. a binder dispersion, for example HPMC) onto seed cores in a fluidized bed such as a Wurster coater or a rotary processor. The seed core can be comprised of a sugar (for example a non-pareil seed), starch or microcrystalline cellulose, conveniently microcrystalline cellulose. In an embodiment, the inert core comprises sugar spheres mesh 45/60 (250-355 microns). An example of a suitable composition and method is to spray a dispersion of the Compound 1 or a pharmaceutically acceptable salt thereof/binder (e.g. HPMC) composition in water on to the seed core. A delayed release coating is then employed to fabricate the membrane, which is applied over the Compound 1 or a pharmaceutically acceptable salt thereof layered seed cores. In an embodiment, the DR pellets formed by this layering process on a seed core comprise the free base of Compound 1.

Due to the delayed release coating the delayed-release compositions do not undergo any significant disintegration or dispersal in the acidic environment of the stomach after oral administration. Therefore, in an embodiment, the delayed-release composition undergoes less than 5% dissolution in 0.01N HCl after 30 mins at 37° C. using USP2 apparatus. In an embodiment the dissolution rate of the drug from the delayed release composition is 10% or less after 30 minutes from the beginning a dissolution test, which is carried out in accordance with a dissolution test (paddle method) described in the United States Pharmacopoeia (USP) under the conditions that 900 mL of 0.01 N hydrochloric acid is used at 37° C. and the paddle rotation speed is 75 rpm. In an embodiment the dissolution rate of the drug from the immediate release composition is 5% or less after 30 minutes from the beginning a dissolution test, which is carried out in accordance with a dissolution test (paddle method) described in the United States Pharmacopoeia (USP) under the conditions that 900 mL of 0.01 N hydrochloric acid is used at 37° C. and the paddle rotation speed is 75 rpm.

When stirred in acidic aqueous media the delayed release composition according to the present invention takes up only relatively small amounts of acid. In an embodiment, the delayed release composition increases in weight by less than 5% (such as less than 4% or less than 3%) after stirring for 2 hr in 0.1N hydrochloric acid in USP disintegration apparatus.

After oral administration, once the delayed-release composition enters the small intestines and the pH increases above approximately 5.5 the delayed release coating may start to dissolve and the composition will undergo disintegration and dispersal.

In an embodiment, the delayed release composition undergoes substantially complete disintegration in pH 6.8 aqueous media in less than 30 minutes (such as less than 15 minutes) at 37° C. using USP disintegration apparatus. In an embodiment, the delayed release composition undergoes substantially complete disintegration and dispersal in pH 6.8 aqueous media in less than 30 minutes (such as less than 15 minutes) at 37° C. using USP disintegration apparatus.

In an embodiment the delayed release composition undergoes greater than 75% dissolution within 240 minutes from the beginning of a dissolution test, which is carried out in accordance with the USP 2 dissolution test (paddle method) under the conditions that 900 mL of pH 6.8 with 2% CTAB buffer is used, the temperature is 37° C. and the paddle rotation speed is 75 rpm. In an embodiment the delayed release composition undergoes greater than 85% dissolution within 2400 minutes from the beginning of a dissolution test, which is carried out in accordance with the USP 2 dissolution test (paddle method) under the conditions that 900 mL of pH 6.8 with 2% CTAB buffer is used, the temperature is 37° C. and the paddle rotation speed is 75 rpm.

In an embodiment the delayed release composition undergoes greater than 75% dissolution within 240 minutes (such as within 150 minutes, or within 90 minutes) from the beginning of a dissolution test, which is carried out in accordance with the USP 2 dissolution test (paddle method; temperature is 37° C.; paddle rotation speed is 75 rpm) under the conditions that 300 mL of pH 6.8 buffer is used for 30 minutes, followed by 900 mL of pH 6.8 buffer with 2% CTAB.

In an embodiment the delayed release composition undergoes greater than 75% dissolution within 240 minutes (such as within 150 minutes) from the beginning of a dissolution test, which is carried out in accordance with the USP 2 dissolution test (paddle method; temperature is 37° C.; paddle rotation speed is 75 rpm) under the conditions that 900 mL of 0.01N hydrochloric acid is used for 30 minutes, followed by 300 mL of pH 6.8 buffer for 30 minutes, followed by 900 mL of pH 6.8 buffer with 2% CTAB.

The delayed release compositions according to the present invention provide only very low systemic (plasma) levels of Compound 1 after oral dosing. In an embodiment, a delayed release composition according to the present invention provides a geometric mean maximum plasma concentration (Cₘₐₓ) of Compound 1 after oral dosing of less than 10 ng/ml, such as less than 5 ng/ml. In an embodiment, a delayed release composition according to the present invention provides a geometric mean maximum plasma concentration (Cₘₐₓ) of Compound 1 of less than 10 ng/ml, such as less than 5 ng/ml or less than 3 ng/ml, after oral dosing of about 120 mg of Compound 1 or a pharmaceutically acceptable salt thereof. In an embodiment, a delayed release composition according to the present invention provides a geometric mean maximum plasma concentration (Cₘₐₓ) of Compound 1 of less than 10 ng/ml, such as less than 5 ng/ml or less than 3 ng/ml, after oral dosing of the composition comprising at least 120 mg of Compound 1 or a pharmaceutically acceptable salt thereof to a fasted subject. In an embodiment, a delayed release composition according to the present invention provides a geometric mean maximum plasma concentration (Cₘₐₓ) of Compound 1 of less than 10 ng/ml, such as less than 5 ng/ml or less than 4 ng/ml, after oral dosing of the composition comprising at least 240 mg of Compound 1 or a pharmaceutically acceptable salt thereof to a fasted subject. In an embodiment, a delayed release composition according to the present invention provides a geometric mean maximum plasma concentration (Cₘₐₓ) of Compound 1 or a pharmaceutically acceptable salt thereof after oral dosing of 0.5-7.5 ng/ml, such as 1-5 ng/ml, or 2-4 ng/ml, after oral dosing of the composition comprising 120-240 mg of Compound 1 or a pharmaceutically acceptable salt thereof to a subject.

The delayed release compositions according to the present invention advantageously provide good colon tissue exposure of Compound 1 after oral dosing. Colon tissue concentrations can be measured by biopsy as described in the Examples section. In an embodiment, a delayed release composition as described herein provides colonic tissue exposure greater than or equal to the systemic exposure of Compound 1 following oral dosing of the composition to a subject. Advantageously, the delayed release solid compositions as described herein deliver higher median levels of Compound 1 to the colon after oral dosing, than achieved with a corresponding dose formulated as an oral solution. In an embodiment, a delayed release composition as described herein provides median sigmoid colon tissue concentrations of greater than 100 ng/g (such as greater than 200 ng/g) after oral dosing of at least 120 mg of Compound 1 or a pharmaceutically acceptable salt thereof to a fasted subject. In an embodiment, a delayed release composition as described herein provides median rectum colon tissue concentrations of greater than 25 ng/g (such as greater than 40 ng/g) after oral dosing of at least 120 mg of Compound 1 or a pharmaceutically acceptable salt thereof to a fasted subject. In an embodiment, a delayed release composition as described herein provides median sigmoid colon tissue concentrations of greater than 20 ng/g (such as greater than 30 ng/g, greater than 50 ng/g, greater than 100 ng/g, or greater than 200 ng/g) after oral dosing of the composition comprising at least 240 mg of Compound 1 or a pharmaceutically acceptable salt thereof to a fasted subject. In an embodiment, a delayed release composition as described herein provides median rectum colon tissue concentrations of greater than 50 ng/g (such as greater than 75 ng/g, or greater than 100 mg/g) after oral dosing of the composition comprising at least 240 mg of Compound 1 or a pharmaceutically acceptable salt thereof to a fasted subject.

Colonic biopsies may also allow for measurement of the proportion of hypoxia-inducible factor (HIF)-1a positive cells in the biopsy samples both before (baseline) and after (e.g. at day 7) treatment with a delayed release solid composition as described herein. In an embodiment, a delayed release composition as described herein provides a greater than 10% (such as greater than 20%) increase from baseline in the proportion of HIF-1a positive cells in the colon after oral dosing of the composition to a subject. In an embodiment, a delayed release composition as described herein provides a greater than 10% (such as greater than 20%) increase from baseline in the proportion of HIF-1α positive cells in the colon after oral dosing of the composition comprising at least 240 mg of Compound 1 or a pharmaceutically acceptable salt thereof to a subject. In an embodiment, a delayed release composition as described herein provides a greater than 10% (such as greater than 20%, greater than 30%, greater than 40%, or greater than 50%) increase from baseline in the proportion of HIF-1a positive cells in the sigmoid colon tissue after oral dosing of the composition comprising at least 240 mg of Compound 1 or a pharmaceutically acceptable salt thereof to a subject.

The chemical stability of Compound 1 or a pharmaceutically acceptable salt thereof in the solid compositions of the present invention has been found to be very good even after several months under accelerated storage conditions. In an embodiment, the delayed release composition provides less than 5% chemical degradation of Compound 1 by HPLC when stored at 40° C. and 75% RH for 3 months. In an embodiment, the delayed release composition provides less than 5% chemical degradation of Compound 1 by HPLC when stored at 25° C. and 60% RH for 6 months. In an embodiment, the delayed release composition provides less than 2% chemical degradation of Compound 1 by HPLC when stored at 25° C. and 60% RH for 3 months. In an embodiment, the delayed release composition provides less than 0.5% (such as less than 0.25%) of a related impurity having a relative retention time (RRT) of 0.43-0.44 by HPLC (compared to the retention time of Compound 1) when stored at 40° C. and 75% RH for 6 months.

In an embodiment, the delayed-release composition comprises a granulate and the granulate is prepared by a wet granulation method. In an embodiment, the delayed-release composition comprises a granulate which is obtainable by wet granulation. Wet granulation can be carried out by any known wet granulation process, including high-shear wet granulation and fluid bed granulation (see for example, Remington: The Science and Practice of Pharmacy, Edition, 22nd Edition, 2012). Conveniently, the wet granulation is carried out with high shear mixing.

### Processes for Forming Delayed Release Solid Compositions

In a fourth aspect, the present invention provides a process for forming a delayed release solid pharmaceutical composition comprising a granulate according to the third aspect, the process comprising the steps of:
a) mixing Compound 1, or a pharmaceutically acceptable salt thereof, with one or more pharmaceutically acceptable excipients selected from a filler, a diluent, a binder and a disintegrant;
b) adding to the mixture from step a) a wetting agent, water and optionally a binder;
c) subjecting the mixture from step b) to wet granulation;
d) drying the wet granules from step c);
e) milling the dried granules to a particle size diameter of <1 mm;
f) optionally blending the milled granules from step e) with one or more pharmaceutically acceptable excipients selected from a filler, a diluent, a disintegrant and a lubricant; and either:
   g1) compressing the blended granule mixture from step e) or step f) into a tablet;
   h) optionally substantially encapsulating the tablet from step g) in a coating; and
   i) substantially encapsulating the composition from step h) in a delayed release coating; or
   g2) substantially encapsulating the granules from step e) or step f) in a delayed release coating or a delayed release capsule.

Steps a) to f) of the above process correspond to steps a) to f) of the process for forming the immediate release compositions. Therefore, all the embodiments described above for the second aspect in relation to steps a) to f) apply equally to the process for forming a delayed-release composition comprising a granulate according to the fourth aspect of the invention.

In an embodiment, the compression in step g1) gives an average tablet hardness of greater than 7 kp, such as greater than 7.5 kp, greater than 8 kp, greater than 8.5 kp or greater than 9 kp. In an embodiment, the compression in step g1) gives an average tablet hardness of 7-12 kp, such as 7.5-11 kp, 8-11 kp, or 8.5-10 kp.

In an embodiment, the delayed-release tablet composition further comprises an additional sub-coating beneath the delayed release coating. The sub-coating is applied to the composition in step h). In an embodiment, the sub-coating is soluble in aqueous media independent of the pH of the media. In an embodiment, the sub-coating is water-soluble at between pH 1 and 8. In an embodiment the sub-coating is PVA-based. In an embodiment, the coating in step h) is applied to the tablet to a weight gain of 2-6%, such as 3-5%, or about 4%.

In an embodiment, the delayed release capsule or the delayed release coating dissolves at pH values greater than about 5.5. In an embodiment, the delayed release capsule or the delayed release coating dissolves at pH values greater than about 6.0. In an embodiment, the delayed release capsule or the delayed release coating dissolves at pH values greater than about 7.0. Conveniently, the delayed release capsule or the delayed release coating dissolves at about pH 5.5.

In an embodiment, the delayed release capsule or the delayed release coating comprises methyl acrylate-methacrylic acid copolymer, ethyl acrylate-methacrylic acid copolymer, hydroxy propyl methyl cellulose acetate succinate or cellulose acetate phthalate.

In a convenient embodiment, the delayed release coating comprises methyl acrylate-methacrylic acid copolymer or ethyl acrylate-methacrylic acid copolymer. Conveniently, the delayed release capsule or the delayed release coating is selected from Eudragit® L 100-55, Eudragit® FS30D, Eudragit® L100, Eudragit® L 12,5, Eudragit® L30 D-55, Eudragit® S100 and Eudragit® S12,5, such as Eudragit® L 100-55.

In an embodiment, the delayed-release coating in step i) or step g2) is applied to the tablet to a weight gain of greater than 8%. In an embodiment, the delayed-release coating in step i) or step g2) is applied to the tablet to a weight gain of greater than 10%. In an embodiment, the delayed-release coating in step i) or step g2) is applied to the tablet to a weight gain of about 12%. In an embodiment, the delayed-release coating in step i) or step g2) is applied to the tablet to a weight gain of about 14%.

In an alternative embodiment of the fourth aspect, the present invention provides a process for forming a delayed release solid pharmaceutical composition according to the third aspect, wherein the delayed release composition comprises an inert core coated with a drug layer comprising Compound 1 or a pharmaceutically acceptable salt thereof and the drug layer-coated core is coated with a delayed release coating, the process comprising the steps of:
a) providing an inert core (such as sugar spheres);
b) dissolving or suspending Compound 1, or a pharmaceutically acceptable salt thereof, with one or more pharmaceutically acceptable excipients comprising a binder (such as HPMC) and a wetting agent (such as poloxamer 188) in an aqueous media;
c) coating the solution or suspension from step b) onto the inert core;
d) drying the drug layer coated core from step c);
e) optionally sub-coating the dried beads from step d) with a non-functional coating layer (such as HPMC) and drying the sub-coated beads;
f) coating the beads with a delayed release coating; and
g) drying the delayed release beads.

In an embodiment, the coating in steps c), e) and f) are carried out by spray-coating. Conveniently, the spray coating is carried out in a fluidized bed such as a Wurster coater or a rotary processor.

Conveniently, the delayed release coating applied in step f) is a delayed release coating as described herein. In an embodiment, the delayed release pellet coating is selected from Eudragit® L 100-55, Eudragit® FS30D, Eudragit® L100, Eudragit® L 12,5, Eudragit® L30 D-55, Eudragit® S100 and Eudragit® S12,5, such as Eudragit® L 100-55. More conveniently, the delayed release pellet coating is selected from Eudragit® FS30D and Eudragit® L30 D-55.

In an embodiment, the delayed release pellets or beads comprise the delayed release coating in an amount ranging from about 10% to about 40% (w/w) of the total weight of the DR pellet.

In an embodiment, there is provided a delayed release solid pharmaceutical composition obtainable by, obtained by or directly obtained by a process according to the fourth aspect of the invention.

### Therapeutic Uses

In a fifth aspect, the present invention provides an immediate release solid pharmaceutical composition according to the first aspect, or a delayed-release solid pharmaceutical composition according to the third aspect, for use in the treatment of diseases or conditions mediated alone, or in part, by PHD.

Conveniently, there is provided an immediate release solid pharmaceutical composition according to the first aspect, or a delayed-release solid pharmaceutical composition according to the third aspect, for use in the treatment of inflammatory bowel disease. Conveniently, there is provided an immediate release solid pharmaceutical composition according to the first aspect, or a delayed-release solid pharmaceutical composition according to the third aspect, for use in the treatment of ulcerative colitis or Crohn's disease.

The present invention provides a method of treating diseases or conditions mediated alone, or in part, by PHD, the method comprising administering to a subject a therapeutically effective amount of an immediate release solid pharmaceutical composition according to the first aspect, or a delayed-release solid pharmaceutical composition according to the third aspect. Conveniently, there is provided a method of treating an inflammatory bowel disease, the method comprising administering to a subject a therapeutically effective amount of an immediate release solid pharmaceutical composition according to the first aspect, or a delayed-release solid pharmaceutical composition according to the third aspect. Conveniently, there is provided a method of treating ulcerative colitis or Crohn's disease, the method comprising administering to a subject a therapeutically effective amount of an immediate release solid pharmaceutical composition according to the first aspect, or a delayed-release solid pharmaceutical composition according to the third aspect.

The present invention provides for the use of an immediate release solid pharmaceutical composition according to the first aspect, or a delayed-release solid pharmaceutical composition according to the third aspect, in the manufacture of a medicament for treating diseases or conditions mediated alone, or in part, by PHD. Conveniently, the disease or condition is inflammatory bowel disease. Conveniently, the disease or condition is ulcerative colitis or Crohn's disease.

In a convenient embodiment of any of the therapeutic uses described herein, the immediate release solid pharmaceutical composition according to the first aspect, or the delayed-release solid pharmaceutical composition according to the third aspect, is administered once or twice daily. In an embodiment, the immediate release solid pharmaceutical composition according to the first aspect, or the delayed-release solid pharmaceutical composition according to the third aspect, is administered orally once daily. In an embodiment, the immediate release solid pharmaceutical composition according to the first aspect, or the delayed-release solid pharmaceutical composition according to the third aspect, is administered orally once daily in the morning. In an embodiment, the immediate release solid pharmaceutical composition according to the first aspect, or the delayed-release solid pharmaceutical composition according to the third aspect, is administered orally twice daily.

In a convenient embodiment of any of the therapeutic uses described herein, the immediate release solid pharmaceutical composition according to the first aspect, or the delayed-release solid pharmaceutical composition according to the third aspect, is administered with food, or up to 30 minutes after a meal. In an embodiment, the immediate release solid pharmaceutical composition according to the first aspect, or the delayed-release solid pharmaceutical composition according to the third aspect, is administered orally with food, or up to 30 minutes after a meal.

In a convenient embodiment of any of the therapeutic uses described herein, the immediate release solid pharmaceutical composition according to the first aspect, or the delayed-release solid pharmaceutical composition according to the third aspect, is administered orally with food, or up to 30 minutes after a meal, once a day. In an embodiment, the immediate release solid pharmaceutical composition according to the first aspect, or the delayed-release solid pharmaceutical composition according to the third aspect, is administered orally with food, or up to 30 minutes after a meal, twice a day.

In a convenient embodiment of any of the therapeutic uses described herein, the immediate release solid pharmaceutical composition according to the first aspect, or the delayed-release solid pharmaceutical composition according to the third aspect, is administered orally wherein the total daily dose of Compound 1 or a pharmaceutically acceptable salt thereof is between 100 mg and 1000 mg, such as between 120 mg and 960 mg, between 480 mg and 960 mg, about 480 mg, or about 960 mg. In an embodiment, the immediate release solid pharmaceutical composition according to the first aspect, or the delayed-release solid pharmaceutical composition according to the third aspect, is administered orally wherein the total daily dose of Compound 1 or a pharmaceutically acceptable salt thereof is between 100 mg and 500 mg. In an embodiment, the immediate release solid pharmaceutical composition according to the first aspect, or the delayed-release solid pharmaceutical composition according to the third aspect, is administered orally wherein the total daily dose of Compound 1 or a pharmaceutically acceptable salt thereof is about 120 mg, about 240 mg, or about 480 mg.

In a further embodiment of the fifth aspect, the immediate release solid pharmaceutical composition according to the first aspect, or the delayed-release solid pharmaceutical composition according to the third aspect, may be administered in combination with one or more additional treatments. Conveniently, the composition may be administered in combination with one or more additional treatments for inflammatory bowel disease (ulcerative colitis or Crohn's disease). In an embodiment, the one or more additional treatments are selected from 5-aminosalicylates (5-ASAs) and steroids. In a more convenient embodiment, the 5-ASAs are selected from sulphasalazine, mesalazine and olsalazine. In a more convenient embodiment, the steroids are selected from prednisone and budesonide.

The following statements are not claims but describe certain aspects and embodiments of the present invention:
(1) An optimized high shear wet granulation manufacturing process for Compound 1 as disclosed herein, which process alters the physical characteristics of Compound 1 when combined with other components within the granulation, allowing Compound 1 from uncoated or coated tablets produced with this process to adequately disperse in all media.
(2) Uncoated or coated tablets produced by the process of statement 1.
(3) A coated tablet produced by statement 1, which coated tablet protects Compound 1 from exposure to light.

### EXAMPLES

### Compound 1

### Example 1: Compound 1 Solubility

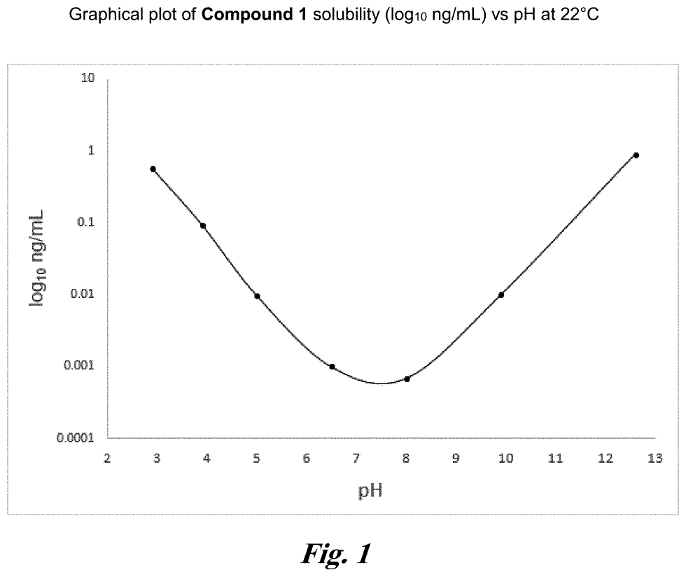

The solubility of Compound 1 at various pH's was investigated. The results are presented in Table 1 and FIG. 1, demonstrating that solubility is pH dependent.

TABLE 1
Compound 1 pH-Solubility at 22° C.

| Compound | Solution | pH | mg/ml |
|---|---|---|---|
| HCl Salt | Water (5 mg/ml load) | 2.2 | 2.07 |
| HCl Salt | Water (1 mg/ml load) | 3.1 | 0.55 |
| HCl Salt | 0.1M HCl | 1.3 | 1.81 |
| HCl Salt | pH 3 (Citrate 25 mM, l = 0.1) | 3.0 | 0.60 |
| Freebase | pH 3 (Citrate 25 mM, l = 0.1) | 2.9 | 0.57 |
| Freebase | pH 4 (Citrate 25 mM, l = 0.1) | 3.9 | 0.094 |
| Freebase | pH 5 (Citrate 25 mM, l = 0.1) | 5.0 | 0.0098 |
| Freebase | pH 6.5 (Phosphate 25 mM, l = 0.1) | 6.5 | 0.0010 |
| Freebase | pH 8 (TRIS 25 mM, l = 0.1) | 8.0 | 0.00069 |
| Freebase | pH 10 (Glycine 25 mM, l = 0.1) | 9.9 | 0.0099 |
| Freebase | 0.1M NaOH | 12.6 | 0.89 |

### Example 2: Formation of Compound 1 HCl Salt

### 2.1 Monohydrate (Form A)

Compound 1 free base (1.0 equiv.) and THF (4 vol.) were charged to a reactor and the contents were agitated for 20 minutes and then cooled to 3° C. 1 M aq. HCl solution (1.5 equiv) was slowly charged for 30 minutes while maintaining the internal temperature of 0-6° C. The mixture was agitated for 20 minutes at 0-6° C. The contents were filtered and washed with aq. THF solution (0.5 vol. of THF/1 vol. of purified water) and purified water (6 vol.). The mixture was distilled under vacuum until target volume was achieved of approximately 10.0 L/kg (with respect to the free base) while maintaining the internal temperature of less than 25° C. The slurry was adjusted to 10° C. and agitated for 1 hour. The product slurry was filtered and the wet cake was washed with pre-cooled (10° C.) purified water (3×1 vol.). The filtered cake was dried under vacuum at less than 35° C.

HPLC analysis of Example 2.1 showed a purity of 99.6% by area. LC-MS demonstrated an observed a mass of 434.2 m/z+ve ionization, consistent with the expected mass of 433.93 g/mol. High Performance Liquid Chromatography-Charged Aerosol Detection (HPLC-CAD) analysis confirmed Example 2.1 to be a mono-HCl salt.

Thermogravimetric analysis of Example 2.1 showed an initial mass loss (from onset to ca. 80° C.) of 3.4% related to loss of water (ca. 0.9 equiv). A loss of ca. 1 equivalent of water indicated Example 2.1 is a monohydrate. A second mass loss of 40.9% was observed (with an onset at ca. 160° C.) related to the material melt and subsequent decomposition. Differential thermal analysis showed a shallow, broad endothermic event (onset at ca. 53° C.) related to the loss of water. An initially sharp then broadening melt was recorded from an onset of ca. 190° C. and a peak at ca. 194° C. DSC analysis showed a broad endothermic event with onset at ca. 70° C. related to the loss of water during the first heating cycle (up to 150° C.), as well as a sharp melt endothermic event with an onset at 191° C. and peak at 194° C. during the second heating cycle (up to 300° C.). The melting point was consistent with TG/DTA.

HPLC analysis of Example 2.1 showed a purity of 99.6% by area. LC-MS demonstrated an observed a mass of 434.2 m/z+ve ionization, consistent with the expected mass of 433.93 g/mol. High Performance Liquid Chromatography-Charged Aerosol Detection (HPLC-CAD) analysis confirmed Example 2.1 to be a mono-HCl salt.

XRPD analysis was carried out on a PANalytical X'pert pro fitted with PIXcel detector, scanning the samples between 3 and 35° 2θ. The material was gently ground to release any agglomerates and loaded onto a multi-well plate with Kapton or Mylar polymer film to support the sample. The multi-well plate was then placed into the diffractometer and analyzed using Cu K radiation (α1λ=1.54060 Å; α2=1.54443 Å; β=1.39225 Å; α1:α2 ratio=0.5) running in transmission mode (step size 0.0130′ 20) using 40 kV/40 mA generator settings.

XRPD characterisation of Example 2.1 gave the peak list presented in Table 2 (referred to as Form A). Polarized Light Microscopy (PLM) showed small birefringent crystals (ca. 20-100 μm) with plate-like morphology.

TABLE 2
XRPD peak list of Example 2.1 (Form A)

| No. | Pos. [2θ] | Area [cts* 2θ] | d-spacing [Å] | Height [cts] | Rel. Int. [%] |
|---|---|---|---|---|---|
| 1 | 5.91200 | 50.07 | 14.94968 | 793.52 | 21.30 |
| 2 | 15.00140 | 93.68 | 5.90583 | 1856.06 | 49.81 |
| 3 | 15.11510 | 259.59 | 5.86166 | 2938.83 | 78.87 |
| 4 | 15.34350 | 116.84 | 5.77491 | 1322.78 | 35.50 |
| 5 | 15.60160 | 89.41 | 5.67997 | 885.73 | 23.77 |
| 6 | 15.93380 | 44.34 | 5.56228 | 702.71 | 18.86 |
| 7 | 16.23020 | 161.42 | 5.46135 | 1599.02 | 42.91 |
| 8 | 17.27050 | 114.45 | 5.13467 | 1511.64 | 40.57 |
| 9 | 17.37160 | 166.49 | 5.10501 | 2198.95 | 59.01 |
| 10 | 19.77420 | 417.34 | 4.48983 | 3674.85 | 98.62 |
| 11 | 20.04290 | 470.19 | 4.43025 | 3726.15 | 100.00 |
| 12 | 20.57610 | 241.80 | 4.31663 | 2129.11 | 57.14 |
| 13 | 20.76170 | 54.15 | 4.27845 | 715.23 | 19.19 |
| 14 | 21.58470 | 97.17 | 4.11715 | 700.03 | 18.79 |
| 15 | 23.15250 | 59.46 | 3.84178 | 673.16 | 18.07 |
| 16 | 25.75900 | 120.64 | 3.45864 | 1195.02 | 32.07 |
| 17 | 25.92860 | 178.40 | 3.43641 | 1570.90 | 42.16 |
| 18 | 26.53970 | 148.49 | 3.35866 | 840.55 | 22.56 |
| 19 | 26.85890 | 102.20 | 3.31945 | 809.91 | 21.74 |
| 20 | 30.12340 | 136.45 | 2.96675 | 831.79 | 22.32 |

### 2.2 Anhydrous (Form B)

Compound 1 HCl salt (1 eq.; Example 2.1) and MeOH (5.5 vol.) were charged to a reactor and agitated at 20-25° C. for 10 minutes. The resulting beige slurry was then heated to 40° C. resulting in a clear, orange solution after 5-10 minutes. MTBE (8.5 vol) was then added over 2 hours at a flow rate of 14.6 mL/minute. Following complete addition, the solution was slowly cooled to 2-7° C. at a rate of 0.1° C./minute. A pale beige slurry was observed and allowed to stir at this temperature for 16 hours. The resulting slurry was filtered, washed with MTBE (2×1 vol.), and dried at 40° C. under vacuum to afford a crystalline solid.

HPLC showed the material is 99.9% pure. PLM analysis showed the material to have a form of fine (ca. 5-20 μm), birefringent crystals with irregular morphology and agglomeration. TG analysis showed no solvent related mass loss, which correlates with Example 2.2 being an anhydrous form. DT analysis showed a sharp melting endotherm with an onset at ca. 193° C. and peak at ca. 195° C., followed by thermal decomposition. DSC analysis showed no significant endothermic events related to solvent loss. An intense, sharp melting endotherm was observed with an onset at ca. 196° C. and peak at ca. 198° C.

XRPD characterisation of Example 2.2 gave the peak list presented in Table 3 (referred to as Form B).

TABLE 3
XRPD peak list of Example 2.2 (Form B)

| No. | Pos. (°2θ) | Area (cts*°2θ) | d-spacing (Å) | Height (cts) | Rel. Int. (%) |
|---|---|---|---|---|---|
| 1 | 4.8011 | 86.98 | 18.4058 | 1378.52 | 50.34 |
| 2 | 9.0409 | 129.05 | 9.7817 | 1460.99 | 53.36 |
| 3 | 9.6207 | 23.67 | 9.1934 | 468.86 | 17.12 |
| 4 | 14.9523 | 49.51 | 5.9251 | 560.46 | 20.47 |
| 5 | 15.2101 | 221.09 | 5.8253 | 1752.07 | 63.99 |
| 6 | 15.6091 | 54.77 | 5.6772 | 620.02 | 22.64 |
| 7 | 16.8437 | 380.08 | 5.2638 | 2738.23 | 100.00 |
| 8 | 18.6186 | 154.99 | 4.7658 | 1754.70 | 64.08 |
| 9 | 18.7535 | 91.76 | 4.7318 | 1211.99 | 44.26 |
| 10 | 19.9168 | 121.58 | 4.4580 | 1204.39 | 43.98 |
| 11 | 20.2725 | 146.63 | 4.3806 | 1452.47 | 53.04 |
| 12 | 20.5119 | 87.99 | 4.3300 | 996.09 | 36.38 |
| 13 | 20.6854 | 72.89 | 4.2941 | 1155.22 | 42.19 |
| 14 | 21.0020 | 65.42 | 4.2301 | 648.00 | 23.66 |
| 15 | 21.7375 | 50.54 | 4.0886 | 445.05 | 16.25 |
| 16 | 22.5631 | 164.83 | 3.9408 | 1306.24 | 47.70 |
| 17 | 24.6591 | 51.68 | 3.6104 | 511.97 | 18.70 |
| 18 | 25.2339 | 76.06 | 3.5294 | 861.03 | 31.44 |
| 19 | 27.3588 | 63.32 | 3.2599 | 456.14 | 16.66 |
| 20 | 29.0781 | 44.47 | 3.0710 | 587.31 | 21.45 |

### Example 3: Excipient Compatibility with Compound 1 HCl Salt

Binary mixtures of Compound 1 HCl salt (prepared according to Example 2.1) and excipient were stored at 40° C. and 75% RH for 4 weeks and analysed at weekly intervals for appearance, moisture content (KF) and HPLC purity of Compound 1, with the excipients studied being microcrystalline cellulose, silicified microcrystalline cellulose, lactose, crospovidone, croscarmellose sodium, sodium starch glycolate, magnesium stearate, sodium stearyl fumarate, red iron oxide, blue #2, eudragit, polyvinylpyrrolidone, HPMC-AS, anhydrous citric acid and HPMC.

While Compound 1 HCl salt was stable at 40° C. and 75% RH for 4 weeks, as were the binary mixtures with most of the excipients, the mixture with citric acid liquefied after 1 week and after 4 weeks the API purity was only 34.1% by HPLC, highlighting that Compound 1 HCl salt is chemically incompatible with citric acid. A slight increase in impurity levels was also seen after 4 weeks in the binary mixtures with microcrystalline cellulose (0.51% total impurities) and silicified microcrystalline cellulose (0.69% total impurities).

### Tablet Formulation Trials

### Direct Compression Preparations

### Example 4: Formulation (F1)

Tablets containing Compound 1⋅HCl (65.17 mg; prepared according to Example 2.1) and the ingredients listed in Table 2, were prepared by blending and compression (8 mm diameter round tooling) to produce plain, white to off white, 8 mm diameter, round, biconvex tablets (F1). Physical parameters of the tablets are presented in Table 3 and show (i) a high spread in tablet weights, possibly due to flow issues, and (ii) long disintegration times possibly due to one of the formulation components (e.g. Eudragit EPO).

TABLE 2
Compound 1 Formulation (F1)

| F1 Ingredients | mg/tab | % (w/w) |
|---|---|---|
| Compound 1 · HCl | 65.17 | 27.15 |
| Microcrystalline Cellulose | 88.85 | 37.02 |
| Lactose Monohydrate SD | 65.59 | 27.33 |
| Eudragit EPO | 12.00 | 5.00 |
| Crospovidone | 4.80 | 2.00 |
| Sodium Lauryl Sulfate | 1.20 | 0.50 |
| Magnesium Stearate | 2.40 | 1.00 |
| Total | 240.00 | 100.00 |

TABLE 3
F1 Physical Parameters

| Parameter | Observation | |
|---|---|---|
| Weight Max | 253.0 | mg |
| Weight Min | 227.0 | mg |
| Weight Avg (10 tabs) | 240.0 | mg |
| Hardness | 11.2 | kp |

| | |
|---|---|
| Friability | 0.34% |
| D.T. (water) | First: 30 min |
| | Last: 45 min |

### Example 5: Formulation (F2)

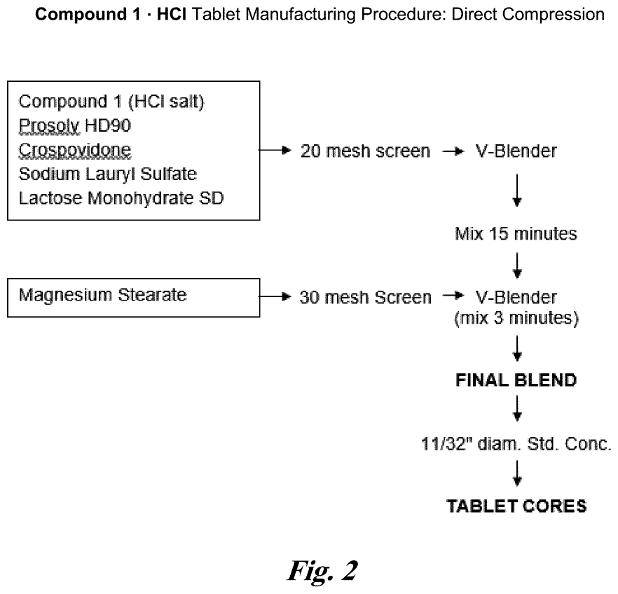

Tablets containing Compound 1⋅HCl (65.17 mg; prepared according to Example 2.1) and the ingredients listed in Table 4, were prepared using a direct compression process as described in FIG. 2.

TABLE 4
Compound 1 Formulation (F2)

| F2 Ingredients | mg/tab | % (w/w) |
|---|---|---|
| Compound 1 · HCl | 65.17 | 27.15 |
| Prosolv HD90 | 100.84 | 42.00 |
| Lactose Monohydrate | 65.59 | 27.33 |
| SD | | |
| Crospovidone | 4.80 | 2.00 |
| Sodium Lauryl Sulfate | 1.20 | 0.50 |
| Magnesium Stearate | 2.40 | 1.00 |
| Total | 240.00 | 100.00 |

The following observations were made on the F2 tablets:
(i) Tablets could not be compressed beyond a hardness of approximately 8-9 kp due to capping and lamination issues experienced at higher compression settings.
(ii) Removal of Eudragit EPO decreased the disintegration time of the tablets from over 30 minutes to less than 1 minute.
(iii) Use of Prosolv HD 90 in place of microcrystalline cellulose improved weight uniformity and decreased friability values.

However, tablet compressibility was limited to <9 kp with this formulation.

Eudragit EPO can be used to inhibit the precipitation of poorly water-soluble drugs by stabilizing the supersaturation of the drug (Bevernage et al., Mol. Pharm. (2011) 564-570; Gao et al., AAPS J. (2012) 703-713). Therefore, its presence in formulation F1 might have been expected to be beneficial to the disintegration and dispersal of the F1 tablets. However, on the basis of the apparent negative impact of Eudragit EPO on tablet disintegration (observed by comparing the F2 and F1 disintegration behavior), other potential precipitation inhibitors such as hydroxypropyl methyl cellulose (HPMC) or polyvinylpyrrolidone (PVP) were not investigated.

### Example 6: Formulations (F3, F4, F5, F6

Tablets containing Compound 1⋅HCl (prepared according to Example 2.1) and the ingredients listed in Table 5, were prepared, without issues, using a direct compression process as described in FIG. 2. The total tablet weight was increased from 240 mg (F1 & F2) to 300 mg (increased amounts of Prosolv HD90) which improved weight uniformity and reduced tablet friability. The tablets were compressible to hardness values of approximately 18 kp without encountering capping or lamination issues. Formulations F3, F4 and F5 were prepared on a larger scale to provide tablets for further examination.

TABLE 5
Compound 1 Formulations (F3, F4, F5, F6)

| Formulation | F3 | | F4 | | F5 | | F6 | |
|---|---|---|---|---|---|---|---|---|
| Batch Size (g) | 300 | | 1000 | | 1000 | | 900 | |
| Ingredients | mg/tab | % | mg/tab | % | mg/tab | % | mg/tab | % |
| Compound 1 • HCl | 65.2 | 21.7 | 67.7 | 22.6 | 67.7 | 22.6 | 67.7 | 22.6 |
| Sodium Lauryl Sulfate | 1.5 | 0.5 | 1.5 | 0.5 | 1.5 | 0.5 | 1.5 | 0.5 |
| ProsolvHD90 | 142.3 | 47.4 | 139.8 | 46.6 | 139.8 | 46.6 | 139.8 | 46.6 |
| Lactose Monohydrate SD | 82.0 | 27.3 | 82.0 | 27.3 | 82.0 | 27.3 | 82.0 | 27.3 |
| Crospovidone | 6.0 | 2.0 | 6.0 | 2.0 | 6.0 | 2.0 | 6.0 | 2.0 |
| Magnesium Stearate | 3.0 | 1.0 | 3.0 | 1.0 | 3.0 | 1.0 | 3.0 | 1.0 |
| Core Total: | 300.0 | 100 | 300.0 | 100 | 300.0 | 100 | 300.0 | 100 |

### Example 7: Sub-Coating and Enteric Coating of F4 Tablets—F4-SC1, F4-SC1-EC1

F4 tablets were sub-coated with Colorcon's Opadry Clear 03K19229 to a weight gain of 4%, to produce F4-SC1 tablets. An enteric coating was then applied to the F4-SC1 tablets with Colorcon's Acryl-Eze White enteric coating system, containing Evonik's Eudragit's L-100-55 methacrylic acid co-polymer, (which solubilizes at pH≥5.5), to a weight gain of 8%, to produce F4-SC1-EC1 tablets. The F4 core and coating components are provided in Table 6.

TABLE 6
Coating of F4 tablets (Sub-Coat SC1 & Enteric Coat EC1)

| | mg/tab | % |
|---|---|---|
| F4 Ingredients | | |
| Compound 1 · HCl | 67.7 | 22.6 |
| Sodium Lauryl Sulfate | 1.5 | 0.5 |
| ProsolvHD90 | 139.8 | 46.6 |
| Lactose Monohydrate SD | 82.0 | 27.3 |
| Crospovidone | 6.0 | 2.0 |
| Magnesium Stearate | 3.0 | 1.0 |
| Core Total: | 300.0 | 100 |
| SC1 Ingredients | | |
| Opadry Clear 03K19229 | 12.4 | 4.0 |
| Sub Coated Tablet Total: | 312.4 | 104.0 |
| EC1 Ingredients | | |
| Acryl-Eze White 93018359 | 27.0 | 8.0 |
| Enteric Coated Tablet Total: | 339.4 | 108.0 |

Disintegration time in water of the enteric coated tablets ranged from 4.5 min to 5.75 min. Enteric coated tablets placed in disintegration apparatus in 0.1 N HCl for 2 hours showed no change in appearance. Uncoated F4 tablets were examined at T=0 and after several hours light exposure under, during which time the tablets darkened and adopted a “scuffed” or mottled appearance. F4 tablets immediately after sub-coating adopt a slight straw coloring associated with the clear coat. Sub-coated tablets exposed to light became darkened and mottled in appearance. Tablets protected from light did not darken, concluding that tablets containing Compound 1 darken during exposure to light, and thus should be protected from prolonged light exposure.

### Example 8: Immediate Release Coating F5 & F6

F5 tablets were sub-coated with Colorcon's Opadry Clear 03K19229 to a weight gain of 4%, to produce F5-SC1 tablets, which were then coated with Colorcon's Opadry II 85F-18422 White immediate release PVA based film coating to a 3% weight gain, to produce F5-SC1-IRC1 tablets.

F6 tablets were sub-coated with Colorcon's Opadry Clear 03K19229 to a weight gain of 4%, to produce F6-SC1 tablets, which were then coated with Colorcon's Opadry II 85F-18422 White immediate release “PVA based” film coating to a 4% weight gain, to produce F6-SC1-IRC2 tablets. The core and coating formulations associated with these trials are provided in Table 7.

TABLE 7
Immediate Release Tablets F5-SC1-IRC1 and F6-SC1-IRC2 (1) F5
(Sub-Coat SC1 & IR Coat IRC1)
and (2) F6 (Sub-Coat SC1 & IR Coat IRC2)

| | Core Tablet | | | |
|---|---|---|---|---|
| | F5 | | F6 | |
| Batch Size (g) | 1000 | | 900 | |
| Core Ingredients | mg/tab | % | mg/tab | % |
| Compound 1 · HCl | 67.7 | 22.6 | 67.7 | 22.6 |
| Sodium Lauryl Sulfate | 1.5 | 0.5 | 1.5 | 0.5 |
| ProsolvHD90 | 139.8 | 46.6 | 139.8 | 46.6 |
| Lactose Monohydrate SD | 82.0 | 27.3 | 82.0 | 27.3 |
| Crospovidone | 6.0 | 2.0 | 6.0 | 2.0 |
| Magnesium Stearate | 3.0 | 1.0 | 3.0 | 1.0 |
| Core Total: | 300.0 | 100 | 300.0 | 100 |
| | Sub Coat | | | |
| | F5-SC1 | | F6-SC1 | |
| SC1 Ingredients | mg/tab | % | mg/tab | % |
| Opadry Clear 03K19229 | 12.4 | 4.0 | 12.4 | 4.0 |
| Sub Coated Tablet Total: | 312.4 | 104.0 | 312.4 | 104.0 |
| | Immediate Release Coat | | | |
| | F5-SC1-IRC1 | | F6-SC1-IRC2 | |
| IRC Ingredients | mg/tab | % | mg/tab | % |
| Opadry II White 85F18422 | 9.8 | 3.0 | 13.0 | 4.0 |
| IR Coated Tablet Total: | 322.2 | 103.0 | 325.4 | 104.0 |

### F5-SC1-IRC1 and F6-SC1-IRC2—Dissolution and Disintegration Studies

Dissolution of F6-SC1-IRC2 Tablets was performed in a USP dissolution apparatus, in 0.01N HCl media. Compound 1 was found to rapidly and fully dissolve (94% in 10 minutes, 98% in 15 minutes).

Dissolution of F6-SC1-IRC2 Tablets was performed in a USP dissolution apparatus, in pH 6.8 phosphate buffer. Tablet disintegration was slow with many large agglomerates remaining intact, and significant coning of material at the bottom of the vessels.

F4-SC1-EC1 tablets in pH 6.8 buffer following the acid phase demonstrated even slower and incomplete disintegration characteristics.

F6-SC1-IRC2 tablets were evaluated in the USP disintegration apparatus, in pH 6.8 phosphate buffer. Tablet disintegration was similar (slightly longer) to that observed in water.

F6-SC1-IRC2 tablets were evaluated in the USP dissolution apparatus (a better mimic of in vivo performance), in pH 6.8 phosphate buffer. Tablets exhibited extended disintegration times.

Compound 1 demonstrated rapid dissolution characteristics in acidic media (see example 1), but is totally insoluble at pH 6.8.

### Example 9: Formulations F7-F11

To address the challenges of tablet disintegration and dispersion of the tablet contents, various formulation modifications were examined, including:
Increasing disintegrant level (crospovidone levels increased from 2% to 5-7%)
Removing Sodium Lauryl Sulfate (absent from F8 & F9; 0.5% in F7, F10 & F11)
Adding citric acid to produce an acidic micro-environment within the tablet (7.5-10% citric acid in F9-F11)
Diluting the API in the formulation by increasing tablet weight from 300 mg to 400 mg (F10 & F11 400 mg tablet cores)

A series of modified core tablets (F7-F11) comprising Compound 1⋅HCl (prepared according to Example 2.1) were prepared as summarized in Table 8, via the same direct compression processes to provide plain, round, biconvex, white to off white tablets. The physical characteristics of these tablets are provided in Table 9.

TABLE 8
Compound 1 Formulations (F7-F11)

| Formulation | F7 | | F8 | | F9 | | F10 | | F11 | |
|---|---|---|---|---|---|---|---|---|---|---|
| Batch Size (g) | 200 | | 1000 | | 300 | | 300 | | 1000 | |
| Ingredients | mg/tab | % | mg/tab | % | mg/tab | % | mg/tab | % | mg/tab | % |
| Compound 1 • HCl | 65.2 | 21.7 | 65.2 | 21.7 | 65.17 | 21.7 | 65.2 | 16.3 | 65.2 | 16.3 |
| Citric Acid | — | — | — | — | 30 | 10 | 30.0 | 7.5 | 30.0 | 7.5 |
| Sodium Lauryl | 1.5 | 0.5 | — | — | — | — | 2.0 | 0.5 | 2.0 | 0.5 |
| Sulfate | | | | | | | | | | |
| ProsolvHD90 | 132.9 | 44.3 | 134.8 | 44.9 | 116.18 | 38.7 | 172.9 | 43.2 | 172.9 | 43.2 |
| Lactose | 76.4 | 25.5 | 82.0 | 27.3 | 70.65 | 23.6 | 106.0 | 26.5 | 106.0 | 26.5 |
| Monohydrate | SD | | | | | | | | | |
| Crospovidone | 21.0 | 7.0 | 15.0 | 5.0 | 15 | 5.0 | 20.0 | 5.0 | 20.0 | 5.0 |
| Magnesium | 3.0 | 1.0 | 3.0 | 1.0 | 3 | 1.0 | 4.0 | 1.0 | 4.0 | 1.0 |
| Stearate | | | | | | | | | | |
| Core Total: | 300.0 | 100 | 300.0 | 100 | 300.0 | 100 | 400.0 | 100 | 400.0 | 100 |

TABLE 9
Physical Characteristics of Formulations (F7-F11)

| Formulation | F7 | F8 | F9 | F10 | F11 |
|---|---|---|---|---|---|
| 1 Diameter | 11/32″ | 11/32″ | 11/32″ * | 10 mm | 10 mm |
| % Friability | 0.11 | 0.09 | Capping | 0.09 | 0.09 |
| Disintegration | First: 1.5 | First: 1.1 | N/A | First: 2.2 | First: 2.2 |
| Time in | | | | | |
| Water (min) | Last: 2.3 | Last: 1.7 | | Last: 2.9 | Last: 3.0 |
| Disintegration | First: 2.9 | First: 1.2 | N/A | First: 2.3 | First: 1.9 |
| Time in | | | | | |
| pH 6.8 Buffer | Last: 2.3 | Last: 2.0 | | Last: 2.8 | Last: 2.3 |
| (min) | | | | | |

* Started with 3/8″round tooling but experienced significant capping

Table 9 demonstrates the disintegration times did not significantly differ between water or pH 6.8 buffer. The 400 mg weight tablets exhibited a longer disintegration time compared to the 300 mg tablets. Addition of citric acid did not provide improvement in disintegration performance. Omission of sodium lauryl sulfate from the formulation showed no noticeable difference in disintegration performance.

### Example 10: Sub-Coating and Enteric Coating of F8 and F11 Tablets—F8-SC2, F8-SC2-EC2, F11-SC2, F11-SC2-EC2

F8 tablets were sub-coated with Colorcon's Opadry Clear 03K19229 to a weight gain of 3%, to produce F8-SC2 tablets. An enteric coating of Colorcon's Acryl-Eze White 93018359 was then applied to the F8-SC2 tablets, to a weight gain of 12%, to produce F8-SC2-EC2 tablets.

F11 tablets were sub-coated with Colorcon's Opadry Clear 03K19229 to a weight gain of 3%, to produce F11-SC2 tablets. An enteric coating was then applied to the F11-SC2 tablets Colorcon's Acryl-Eze White 93018359, to a weight gain of 12%, to produce F11-SC2-EC2 tablets

The core and coating components are provided in Table 10.

TABLE 10
Coating of F8 and F11 core tablets

| | Formulation | | | |
|---|---|---|---|---|
| | F8 | | F11 | |
| Batch Size (g) | 1000 | | 1000 | |
| Core Ingredients | mg/tab | % | mg/tab | % |
| Compound 1 · HCl | 65.2 | 21.7 | 65.2 | 16.3 |
| Citric Acid | — | — | 30.0 | 7.5 |
| Sodium Lauryl Sulfate | — | — | 2.0 | 0.5 |
| ProsolvHD90 | 134.8 | 44.9 | 172.9 | 43.2 |
| Lactose Monohydrate SD | 82.0 | 27.3 | 106.0 | 26.5 |
| Crospovidone | 15.0 | 5.0 | 20.0 | 5.0 |
| Magnesium Stearate | 3.0 | 1.0 | 4.0 | 1.0 |
| Core Total: | 300.0 | 100 | 400.0 | 100 |
| | Sub Coat SC2 | | | |
| | F8-SC2 | | F11-SC2 | |
| SC2 Ingredients | mg/tab | % | mg/tab | % |
| Opadry Clear 03K19229 | 9.3 | 3.0 | 12.3 | 3.0 |
| Sub Coated Tablet Total: | 309.3 | 103.0 | 412.3 | 103.0 |
| | Enteric Coat EC2 | | | |
| | F8-SC2-EC2 | | F11-SC2-EC2 | |
| EC2 Ingredients | mg/tab | % | mg/tab | % |
| Acryl-Eze White 93018359 | 42.0 | 12.0 | 56.0 | 12.0 |
| Enteric Coated Tablet Total: | 351.3 | 12.0 | 468.3 | 112.0 |

Enteric coated tablets, F8-SC2-EC2 and F11-SC2-EC2, were immersed in 0N HCl, in a USP disintegration apparatus. After 2 hours all of the tablets remained intact. They were removed from the apparatus and the acid uptake measured. The tablets were returned to the disintegration apparatus and placed in pH 6.8 Phosphate Buffer. All tablets disintegrated within 3.5-12 minutes (F8-SC2-EC2) or 15-20 minutes (F11-SC2-EC2). For F8-SC2-EC2 tablets, significant soft agglomerates remained that failed to disintegrate by 30 minutes. The disintegration performance observations are presented in Table 11 and Table 12.

TABLE 11
Enteric Coated Tablets F8-SC2-EC2 in 0.1N HCl and pH 6.8
Phosphate Buffer

| | | | | | |
|---|---|---|---|---|---|
| % Enteric Coating | 3 | 5 | 7 | 9 | 12 |
| % Acid Uptake | 39.0 | 12.7 | 5.1 | 4.2 | 2.7 |
| Appearance | mostly | 1/6 tabs | un- | un- | un- |
| after 2 Hrs 0.1N HCl | swollen | swollen | changed | changed | changed |
| Disintegration times | 3.5-8 | 4 | 5 | 10 | 12 |
| in pH 6.8 Buffer | | | | | |
| (mins) | | | | | |

TABLE 12
Enteric Coated Tablets F11-SC2-EC2 in 0.1N HCl and pH 6.8
Phosphate Buffer

| | | | | |
|---|---|---|---|---|
| % Enteric Coating | 6 | 8 | 10 | 12 |
| % Acid Uptake | 33.4 | 24.2 | 15.9 | 3.3 |
| Appearance | 1 tab | slightly | slightly | un- |
| after 2 Hrs 0.1N HCl | cracked, | swollen | swollen | changed |
| | swollen | | | |
| Disintegration times | 15.5 | 20 | 15 | 18 |
| in pH 6.8 Buffer | | | | |
| (mins) | | | | |
| Agglomerates after | None | Soft | Soft | None |
| 30 mins in pH 6.8 | | agglomerates | agglomerates | |
| Buffer | | remain | remain 1/6 tabs | |

An additional six F11-SC2-EC2 enteric coated tablets (12% weight gain) were acid treated for 2 hours using the DT apparatus and then added to each of the six vessels of USP dissolution apparatus set up with 900 ml of pH 6.8 Phosphate Buffer with paddles rotating at a speed of 75 rpm; vessel #1 also had 1% polysorbate 80 added to the buffer (the remaining vessels did not). All tablets disintegrated within 10 to 16 minutes. Some intact agglomerates remained and were recovered after 45 minutes. Addition of 1% Polysorbate 80 to the buffer did not improve the dispersion characteristics of the tablet.

### Example 11: Evaluation of Poloxamer 188

Compound 1⋅HCl (0.25 g) was added to each of two stirred beakers containing water (50 mL), one also containing poloxamer 188 (0.25 g). The dispersion of Compound 1 throughout the beaker was higher in the presence of Poloxamer 188.

### Example 12: Formulations F12, 13, 14

On the basis of the effect of the wetting agent Poloxamer 188 on the API dispersal in water seen in Example 11, direct compression tablets were prepared containing 5% w/w poloxamer 188.

Tablets containing Compound 1⋅HCl (65.2 mg; prepared according to Example 2.1) and the ingredients listed in Table 13, were prepared by blending and compression (11 mm shallow concave bevel edged compression tooling) as described in FIG. 2 to produce plain, white to off white, round, 11 mm diameter, shallow biconvex, bevel edge tablets (F12, F13, F14). No issues were encountered during the preparation of the blends or during tablet compression.

TABLE 13
Compound 1 Formulations F12, F13, F14 Containing Poloxamer 188

| Formulation | F12 | | F13 | | F14 | |
|---|---|---|---|---|---|---|
| Batch Size (g) | 300 | | 1000 | | 1200 | |
| Ingredients | mg/tab | % | mg/tab | % | mg/tab | % |
| Compound 1 • HCl | 65.2 | 16.3 | 65.2 | 16.3 | 65.2 | 16.3 |
| Citric Acid | 30.0 | 7.5 | 30.0 | 7.5 | | |
| Poloxamer 188 | 20.0 | 5.0 | 20.0 | 5.0 | 20.0 | 5.0 |
| ProsolvHD90 | 161.7 | 40.4 | 161.7 | 40.4 | 191.7 | 47.9 |
| Lactose Monohydrate SD | 99.1 | 24.8 | 99.1 | 24.8 | 99.1 | 24.8 |
| Crospovidone | 20.0 | 5.0 | 20.0 | 5.0 | 20.0 | 5.0 |
| Magnesium Stearate | 4.0 | 1.0 | 4.0 | 1.0 | 4.0 | 1.0 |
| Core Total: | 400.0 | 100.0 | 400.0 | 100.0 | 400.0 | 100.0 |

Measurements of tablet hardness and disintegration times in water and pH 6.8 buffer are presented in Table 14. The disintegration times were similar for all tablets and the use of water vs. pH 6.8 buffer showed no significant differences.

TABLE 14
Physical Characteristics of F12, F13 & F14 Core Tablets

| | Formulation | | |
|---|---|---|---|
| | F12 | F13 | F14 |
| Avg Hardness (kp) | 10.56 | 9.91 | 11.26 |
| Disintegration Time | First: 1.8 | First: 1.9 | First: 1.5 |
| in Water (min) | Last: 2.3 | Last: 2.7 | Last:2.2 |
| Disintegration Time | First: 1.7 | First: 1.8 | First: 1.8 |
| in pH 6.8 Buffer (min) | Last: 2.2 | Last: 2.8 | Last: 2.2 |

### Example 13: Sub-Coating and Enteric Coating of F13 and F14 Tablets—F4-SC1, F4-SC1-EC1

F13 tablets were sub-coated with Colorcon's Opadry Clear 03K19229 to a weight gain of 4%, to produce F13-SC1 tablets. An enteric coating of Colorcon's Acryl-Eze White 93018359 was then applied to the F8-SC2 tablets, to a weight gain of 12.5%, to produce F13-SC1-EC3 tablets.

F14 tablets were sub-coated with Colorcon's Opadry Clear 03K19229 to a weight gain of 4%, to produce F14-SC1 tablets. The core and coating components are provided in Table 15.

TABLE 15
Coating of F13 and F14 core tablets containing Poloxamer 188

| | Formulation | | | |
|---|---|---|---|---|
| | F13 | | F14 | |
| Batch Size (g) | 1000 | | 1200 | |
| Core Ingredients | mg/tab | % | mg/tab | % |
| Compound 1 · HCl | 65.2 | 16.3 | 65.2 | 16.3 |
| Citric Acid | 30.0 | 7.5 | | |
| Poloxamer 188 | 20.0 | 5.0 | 20.0 | 5.0 |
| ProsolvHD90 | 161.7 | 40.4 | 191.7 | 47.9 |
| Lactose Monohydrate SD | 99.1 | 24.8 | 99.1 | 24.8 |
| Crospovidone | 20.0 | 5.0 | 20.0 | 5.0 |
| Magnesium Stearate | 4.0 | 1.0 | 4.0 | 1.0 |
| Core Total: | 400.0 | 100.0 | 400.0 | 100.0 |
| | Sub Coat SC1 | | | |
| | F13-SC1 | | F14-SC1 | |
| SC1 Ingredients | mg/tab | % | mg/tab | % |
| Opadry Clear 03K19229 | 16.8 | 4.0 | 16.8 | 4.0 |
| Sub Coated Tablet Total: | 416.8 | 104.0 | 416.8 | 104.0 |

| Enteric Coat EC3 | F13-SC1-EC3 | |
|---|---|---|
| EC3 Ingredients | mg/tab | % |
| Acryl-Eze White 93018359 | 59.5 | 12.5 |
| Enteric Coated | 476.3 | 112.5 |
| Tablet Total: | | |

Enteric coated tablets, F13-SC1-EC3, were immersed in 0.1N HCl, in a USP disintegration apparatus. After 2 hours all of the tablets remained intact. They were removed from the apparatus and the acid uptake measured. The tablets were returned to the disintegration apparatus and placed in pH 6.8 Phosphate Buffer. All tablets disintegrated within 12-20 minutes. Observations of their disintegration performance are provided in Table 16.

TABLE 16
Enteric Coated Tablets F13-SCI-EC3 in 0.1N
HCI and pH 6.8 Phosphate Buffer

| | | | | |
|---|---|---|---|---|
| % Enteric Coating | 8 | 10 | 11.5 | 12.5 |
| % Acid Uptake | 29.4 | 14.1 | 3.3 | 3.0 |
| Appearance after | slightly | slightly | unchanged | unchanged |
| 2Hrs 0.1N HCI | swollen | swollen | | |
| Disintegration | 12-15 | 20 | 18 | 18 |
| times in pH | | | | |
| 6.8 Buffer (mins) | | | | |
| Agglomerates after | some | Significant | None | None |
| 30 mins in pH 6.8 | | 3/6 tabs | | |
| Buffer | | | | |

Six F13-SC1-EC3 enteric coated tablets (12.5% weight gain) were acid treated and used to evaluate tablet disintegration and dispersion using USP dissolution apparatus (pH 6.8 buffer, 900 ml, paddles, 75 rpm). Generally, it took about 20-30 minutes before all tablets appeared fully disintegrated. After straining the contents of the dissolution vessels, significant amounts of soft agglomerates were recovered.

A disintegration test was performed on the sub-coated F14-SC1 tablets that revealed the sub-coating process had increased the disintegration time to approximately 30 minutes, from 4 minutes for previous sub-coated tablets, not containing poloxamer 188. It appears therefore, that Poloxamer 188 may be extending disintegration times. Additional formulations containing poloxamer 188 were not prepared.

### Example 14: Core Tablets F15 & 16, Sub-Coated F15-SC1 & F16-SC1 and Enteric Coated F16-SC1-EC2

Core tablets F15 & 16 were prepared containing the ingredients presented in Table 17; the level of disintegrant crospovidone was increased from 5% to 10% over previous formulations. Of note, F15 & 16 are identical core formulations that differ only in scale; F15 was prepared to explore the formulation's suitability for manufacture at a larger scale required for coating purposes. The tablets produced were plain, white to off white, round, 11 mm diameter, shallow biconvex and bevel edged in appearance. The core tablets compressed well, with low weight uniformity, good tablet hardness and low tablet friability. Table 18 presents the hardness values and disintegrations times in water and pH 6.8 buffer, showing the tablets disintegrated quickly (1 minute or less) in both media.

F15 & 16 core tablets were sub-coated with Colorcon's Opadry Clear 03K19229 to a weight gain of 4%, to produce F15-SC1 & F16-SC1 tablets.

An enteric coating was then applied to the F16-SC1 tablets with Colorcon's Acryl-Eze 93018359 White enteric coating system, to a weight gain of 12%, to produce F16-SC1-EC2 tablets.

TABLE 17
Core tablets F15 & 16, Sub-Coated F15-SCI & F16-SCI
and Enteric Coated F16-SC1-EC2

| Formulation | F15 | | F16 | |
|---|---|---|---|---|
| Batch Size (g) | 300 | | 1150 | |
| Core Ingredients | mg/tab | % | mg/tab | % |
| Compound 1 • HCI (Ex. 2.1) | 65.2 | 16.3 | 65.2 | 16.3 |
| ProsolvHD90 | 290.8 | 72.7 | 290.8 | 72.7 |
| Crospovidone | 40.0 | 10.0 | 40.0 | 10.0 |
| Magnesium Stearate | 4.0 | 1.0 | 4.0 | 1.0 |
| Core Total: | 400.0 | 100.0 | 400.0 | 100 |
| Sub Coat | F15-SCI | | F16-SCI | |
| SC1 Ingredients | mg/tab | % | mg/tab | % |
| Opadry Clear 03K19229 | 16.8 | 4.0 | 16.8 | 4.0 |
| Sub Coated Tablet Total: | 416.8 | 104.0 | 416.8 | 104.0 |
| Enteric Coating | — | — | F16-SCI-EC2 | |
| EC2 Ingredients | mg/tab | % | mg/tab | % |
| Acryl-Eze White 93018359 | — | — | 57.1 | 12.0 |
| Enteric Coated Tablet Total: | — | — | 473.9 | 112.0 |

TABLE 18
Physical Characteristics
of F15 & F16 Core Tablets

| Formulation | F15 | F16 |
|---|---|---|
| Avg Hardness (kp) | 13.77 | 13.07 |
| Disintegration Time | First: 35 | First: 25 |
| in Water (sec) | Last: 48 | Last: 35 |
| Disintegration Time | First: 45 | First: 23 |
| in pH 6.8 Buffer | Last: 60 | Last: 40 |
| (sec) | | |

Enteric coated tablets F16-SC1-EC2, were immersed in 0.1N HCl, in a USP disintegration apparatus. After 2 hours all of the tablets remained intact. They were removed from the apparatus and the acid uptake measured. The tablets were returned to the disintegration apparatus and placed in pH 6.8 Phosphate Buffer. All tablets disintegrated within 8-10 minutes. The disintegration performance observations are presented in Table 19.

TABLE 19
Enteric Coated Tablets F16-SCI-EC2 in 0.1N
HCI and pH 6.8 Phosphate Buffer

| | | | | |
|---|---|---|---|---|
| % Enteric Coating | 6 | 8 | 10 | 12 |
| % Acid Uptake | 19.6 | 22.1 | 12.0 | 3.2 |
| Appearance | slightly | slightly | unchanged | unchanged |
| after 2Hrs 0.1N HCI | swollen | swollen | | |
| Disintegration times | 9-10 | 8 | 8 | 10 |
| in pH 6.8 Buffer (mins) | | | | |
| Agglomerates after | Some: | Some: | None | None |
| 30 mins in pH 6.8 Buffer | 1/6 tabs | 4/6 tabs | | |

Six tablets of the 12.5% enteric coated sample were acid treated and the tablet disintegration and dispersion properties were evaluated using USP dissolution apparatus (pH 6.8 buffer, 900 ml, paddles, 75 rpm). Generally, it took about 20 minutes for all tablets to appear fully disintegrated. However, after straining the contents of the dissolution vessels, a significant amount of soft agglomerates were recovered from all six vessels. Thus, the dispersion characteristics of the tablets remained unsatisfactory.

### Summary of Direct Compression Trials

The above examples show that the direct compression formulations did not adequately disperse Compound 1 in media in which it is not readily soluble (e.g. pH 6.8 buffer), even following experiments which included:
adding precipitation inhibitors (Example 4);
using acidic buffering agents such as citric acid (Examples 9-10);
diluting the API level in the tablets (Examples 9-10);
replacing SLS with Poloxamer 188 (Examples 12-13); and
using high levels of disintegrant (Example 14);

A direct compression process does not appear to change the physical characteristics of Compound 1⋅HCl within the tablet micro-environment, such that once the coated tablet disintegrates, the liberated hydrophobic Compound 1, in close proximity to itself, agglomerates to resist dispersion.

In conclusion, it was determined that tablets prepared by direct compression of the blended formulations containing Compound 1⋅HCl exhibited unsatisfactory disintegration and dissolution properties.

### High Shear Wet Granulation Preparations

It was surprisingly found that high shear wet granulation processes intimately mix Compound 1 hydrochloride salt with solubility-enhancing excipients, such as surfactants, disintegrants and binders, to form dense homogenous granules suitable for tableting when combined with additional extra-granular components. When a tablet produced by this process disintegrates, the granules containing Compound 1⋅HCl and other excipients are dispersed within the media and subsequently disintegrate themselves to liberate Compound 1 into the media. This two-stage disintegration provides superior dispersion throughout the media and has been found to surprisingly avoid the formation of API agglomerates.

During the direct compression trials wetting agents or surfactants were found to have negative or minimal effects on the disintegration and/or dispersal of Compound 1 hydrochloride salt: the omission of SLS from the formulation did not have a negative impact on these properties; addition of 1% Polysorbate 80 to the pH 6.8 buffer media did not improve the dispersion characteristics, so was not considered for inclusion in the direct compression formulations; and whilst poloxamer 188 was found to improve the aqueous dispersion of Compound 1⋅HCl, nevertheless, when it was incorporated in the direct compression formulations it was found to be hindering tablet disintegration. It was therefore surprising that when the Applicants attempted to use certain wetting agents in granule formulations, they unexpectedly found that certain benefits were observed.

During the wet granulation process a wetting agent may advantageously be used to improve the disintegration and dispersal of Compound 1 hydrochloride salt from the resultant composition. The wetting agent is dissolved in water or an aqueous medium and added to the shear-blended formulation to assist in granulation. It is postulated that when a wetting agent is intimately mixed with Compound 1 hydrochloride salt within a granulate composition, then the two-stage disintegration of the composition and dispersal of the API in aqueous media is promoted.

The wetting agent may be a surfactant or emulsifier. Conveniently the wetting agent is a non-ionic surfactant. Conveniently, the wetting agent has a hydrophilic-lipophilic balance (HLB) between 10 and 25 (such as between 12 and 18). Suitable examples of non-ionic wetting agents include polyol esters, polyoxyethylene esters and poloxamers. Examples of polyol esters include glycol esters, glycerol esters and sorbitan derivatives. Sorbitan derivates comprise polysorbates (such as polysorbate 20, polysorbate 40, polysorbate 60 and polysorbate 80). Most conveniently the wetting agent is polysorbate 80.

### Example 15: Core Tablets 17-21

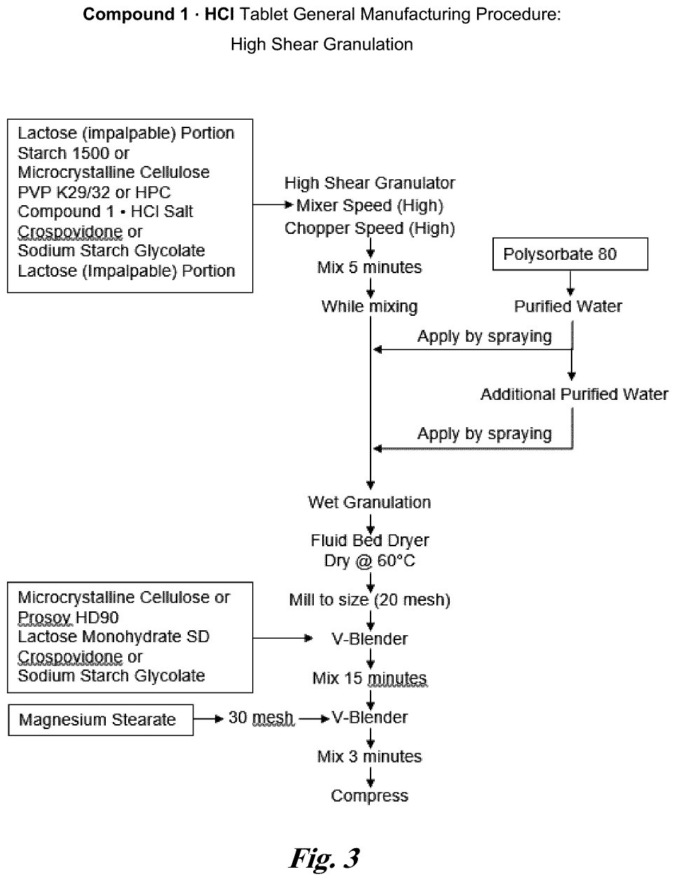

Five small-scale (batch size 250 g) high shear granulation formulations, F17-F21, were prepared according to the core tablet formulations provided in Table 20 and the process described in FIG. 3. Despite no benefit of Polysorbate 80 in direct compression tablets, Polysorbate 80 was introduced as a surfactant intended to assist in wetting Compound 1 HCl in aqueous media in the granule formulations. All formulations were successfully granulated and blended to produce free-flowing granulations suitable for tablet compression. The granulation process improved the flow of the final blend. Formulations F18, F19, F20 & F21 compressed well, while F17 could not be compressed due to insufficient hardness, to produce plain, white to off white, round, 11 mm diameter, shallow biconvex, bevel edge tablet. The weight uniformity of the tablets was good.

Hardness values and disintegration times, in water and pH 6.8 buffer, of formulations F18-F21 are provided in Table 21. Disintegration times increased, as compared to the direct compression formulations, and were similar in water and pH 6.8 buffer.

TABLE 20
High Shear Granulation Formulations F17-F21

| | Formulation | | | | | | | | | |
|---|---|---|---|---|---|---|---|---|---|---|
| | F17 | | F18 | | F19 | | F20 | | F21 | |
| Ingredients | mg/tab | % | mg/tab | % | mg/tab | % | mg/tab | % | mg/tab | % |
| Lactose Monohydrate | 165.0 | 41.3 | 145.0 | 36.3 | 145.0 | 36.3 | 145.0 | 36.3 | 96.4 | 24.1 |
| Starch 1500 | 55.0 | 13.8 | 47.8 | 12.0 | 47.8 | 12.0 | 47.8 | 12.0 | | |
| Microcrystalline Cellulose | | | | | | | | | 96.4 | 24.1 |
| PVP K29/32 | 12.0 | 3.0 | 20.0 | 5.0 | 20.0 | 5.0 | 20.0 | 5.0 | 20.0 | 5.0 |
| Compound 1 • HCl (Ex. 2.1) | 65.2 | 16.3 | 65.2 | 16.3 | 65.2 | 16.3 | 65.2 | 16.3 | 65.2 | 16.3 |
| Crospovidone | | | 12.0 | 3.0 | 12.0 | 3.0 | 12.0 | 3.0 | 12.0 | 3.0 |
| Sodium Starch Glycolate | 24.0 | 6.0 | | | | | | | | |
| Polysorbate 80 | 4.0 | 1.0 | 4.0 | 1.0 | 4.0 | 1.0 | 4.0 | 1.0 | 4.0 | 1.0 |
| Microcrystalline Cellulose | 46.8 | 11.7 | | | | | | | | |
| ProsolvHD90 | | | 90.0 | 22.5 | 90.0 | 22.5 | 90.0 | 22.5 | 90.0 | 22.5 |
| Sodium Starch Glycolate | 24.0 | 6.0 | | | | | | | | |
| Crospovidone | | | 12.0 | 3.0 | 12.0 | 3.0 | 12.0 | 3.0 | 12.0 | 3.0 |
| Magnesium Stearate | 4.0 | 1.0 | 4.0 | 1.0 | 4.0 | 1.0 | 4.0 | 1.0 | 4.0 | 1.0 |
| Core Total: | 400.0 | 100 | 400.0 | 100 | 400.0 | 100 | 400.0 | 100 | 400.0 | 100 |

TABLE 21
F18-F21 Tablet Characteristics

| | | | | |
|---|---|---|---|---|
| Formulation | F18 | F19 | F20 | F21 |
| Avg Hardness (kp) | 9.42 | 8.10 | 8.20 | 8.51 |
| Disintegration Time in Water | First: 8.7 | First: 7.8 | First: 8.7 | First: 6.3 |
| (min) | Last: 9.7 | Last: 8.9 | Last: 10.1 | Last: 6.9 |
| Disintegration Time in pH | First: 7.9 | First: 7.4 | First: 8.3 | First: 5.7 |
| 6.8 Buffer (min) | Last: 9.0 | Last: 8.5 | Last 8.8 | Last: 6.3 |

### Example 16: Core Tablets F22. F23 and F24

Formulations F22, F23 and F24 were prepared as detailed in Table 22 and the process described in FIG. 3. These formulations incorporated the following modifications: using intra-granular microcrystalline cellulose (at approximately twice the level of intra-granular lactose monohydrate); removal of Starch 1500 from the formulation; and using HPC in place of PVP K29/32 as the primary binder. An Opadry Clear 03K19229 sub coat was applied to F23, to produce F23-SC1, which was then enterically coated with Acryl-Eze White 93018359, to produce F23-SC1-EC2. An immediate release Opadry II White 85F18422 coat was applied to F24 to a weight gain of 4%, to produce F24-IRC2.

TABLE 22
Formulations F22, F23 and F24

| Formulation | F22 | | F23 | | F24 | |
|---|---|---|---|---|---|---|
| Batch Size (g): | 250 | | 1100 | | 1000 | |
| Ingredients | mg/tab | % | mg/tab | % | mg/tab | % |
| Compound 1 • | 65.2 | 16.3 | 66.3 | 16.6 | 66.3 | 16.6 |
| HCI (Ex. 2.1) | | | | | | |
| Microcrystalline | 131.9 | 33.0 | 130.8 | 32.7 | 130.8 | 32.7 |
| Cellulose | | | | | | |
| Lactose | 65.9 | 16.5 | 65.9 | 16.5 | 65.9 | 16.5 |
| Monohydrate | | | | | | |
| Crospovidone | 12.0 | 3.0 | 12.0 | 3.0 | 12.0 | 3.0 |
| Hydroxy Propyl | 15.0 | 3.8 | 15.0 | 3.8 | 15.0 | 3.8 |
| Cellulose | | | | | | |
| EXF | | | | | | |
| Polysorbate 80 | 4.0 | 1.0 | 4.0 | 1.0 | 4.0 | 1.0 |
| ProsolvHD90 | 90.0 | 22.5 | 90.0 | 22.5 | 90.0 | 22.5 |
| Crospovidone | 12.0 | 3.0 | 12.0 | 3.0 | 12.0 | 3.0 |
| Magnesium Stearate | 4.0 | 1.0 | 4.0 | 1.0 | 4.0 | 1.0 |
| Core Total: | 400.0 | 100 | 400.0 | 100 | 400.0 | 100 |
| Sub Coat | | | F23-SC1 | | | |
| SC1 Ingredients | | | mg/tab | % | | |
| Opadry Clear | | | 16.8 | 4.0 | | |
| 03K19229 | | | | | | |
| Sub Coated | | | 416.8 | 104.0 | | |
| Tablet Total: | | | | | | |
| Immediate | | | | | F24-IRC2 | |
| Release Coat | | | | | | |
| IRC2 Ingredients | | | | | mg/tab | % |
| Opadry II VVhite | | | | | 16.8 | 4.0 |
| 85F18422 | | | | | | |
| IR Coated : | | | | | 416.8 | 104.0 |
| Tablet Total | | | | | | |
| Enteric Coat EC2 | F23-SC1-EC2 | | | | | |
| EC2 Ingredients | mg/tab | % | | | | |
| Acryl-Eze White | 57.1 | 12.0 | | | | |
| 93018359 | | | | | | |
| Enteric Coated | 473.9 | 12.0 | | | | |
| Tablet Total: | | | | | | |

F23 and F24 were compressed at similar hardness levels to produce plain, white to off-white, round, 11 mm Diam., shallow biconvex, bevel edge tablets. F22 tablets were compressed at three hardness levels to produce plain, white to off white, round, 11 mm diameter, shallow biconvex, bevel edge tablets.

A summary of the physical blend characteristics of the high shear wet granulation formulations F22, F23 and F24 is provided in Table 23. For comparison, a summary of the physical characteristics for the process formulations F4, F5, F6, F8, F11, F14 and F16 which were prepared by direct compression are also included in Table 23.

The bulk densities of the wet granulated formulations (F22-F24) were significantly higher than that of the direct compression blends. As a result, the calculated Carr's Index values and Hausner ratios are significantly lower indicating a free-flowing material. The high shear granulation produced spherical granules with a relatively coarser distribution in comparison to the direct compression blends (33.4-46.1% particles sized 177-420 μm for F22-F24, c.f. 11.4-38.0% for F4-F16; 17.5-19.9% particles sized 125-177 μm for F22-F24, c.f. 40.9-59.9% for F4-F16).

TABLE 23
Physical Blend Characteristics of Formulations F22, F23 and F24 prepared by High Shear Wet Granulation
compared with Formulations F4, F5, F6, F8, F11, F14 and F16 prepared by Direct Compression

| Tests | F22 | F23 | F24 | F4 | F5 | F6 | F8 | F11 | F14 | F16 |
|---|---|---|---|---|---|---|---|---|---|---|
| Bulk Density | 0.543 | 0.538 | 0.532 | 0.410 | 0.417 | 0.395 | 0.385 | 0.421 | 0.427 | 0.371 |
| (g/ml) | | | | | | | | | | |
| Tapped Density | 0.667 | 0.679 | 0.676 | 0.676 | 0.694 | 0.675 | 0.645 | 0.690 | 0.667 | 0.623 |
| (g/ml) | | | | | | | | | | |
| Carr's Index (%) | 18.6 | 20.8 | 21.3 | 39.4 | 39.9 | 41.5 | 40.3 | 39.0 | 36.0 | 40.5 |
| Hausner Ratio: | 1.23 | 1.26 | 1.27 | 1.65 | 1.66 | 1.71 | 1.68 | 1.64 | 1.56 | 1.68 |
| US Mesh Size | | | | | | | | | | |
| (max. particle | | | | | | | | | | |
| size in μm) | % RETAINED | | | | | | | | | |
| 20 (841) | 0.0 | 0.0 | 0.0 | 0.0 | 0.0 | 0.0 | 0.0 | 0.0 | 0.0 | 0.0 |
| 30 (595) | 1.2 | 5.7 | 6.9 | 0.2 | 0.8 | 0.5 | 0.4 | 0.2 | 2.0 | 0.5 |
| 40 (420) | 16.7 | 14.9 | 8.5 | 0.2 | 0.9 | 0.8 | 0.6 | 2.5 | 2.4 | 1.2 |
| 60 (250) | 27.9 | 17.8 | 16.9 | 13.8 | 14.3 | 15.2 | 11.1 | 9.4 | 12.3 | 5.0 |
| 80 (177) | 18.2 | 15.6 | 19.2 | 15.9 | 19.7 | 22.8 | 20.3 | 9.7 | 12.8 | 6.4 |
| 100 (149) | 10.0 | 11.8 | 11.8 | 42.6 | 43.5 | 43.6 | 45.0 | 33.1 | 33.9 | 52.5 |
| 120 (125) | 7.5 | 7.4 | 8.1 | 7.3 | 2.8 | 1.9 | 4.7 | 8.3 | 7.0 | 7.4 |
| 170 (88) | 9.4 | 14.9 | 14.4 | 14.4 | 11.7 | 10.2 | 14.6 | 29.6 | 23.0 | 20.5 |
| 200 (74) | 3.4 | 3.6 | 5.4 | 2.4 | 2.6 | 2.0 | 1.3 | 3.4 | 4.8 | 4.5 |
| 230 (63) | 1.9 | 3.0 | 3.4 | 0.5 | 0.8 | 0.7 | 0.5 | 1.0 | 0.6 | 0.5 |
| Pan | 3.8 | 5.3 | 5.2 | 1.8 | 2.8 | 1.3 | 1.5 | 1.7 | 1.2 | 1.5 |

Physical Characteristics of F22, F23, F24 Tablets are presented in Table 24. Tablet friability was similar at all hardness values. Disintegration times varied significantly, increasing as hardness increased.

TABLE 24
Physical Characteristics of F22, F23, F24 Tablets

| | | F22 | | | |
|---|---|---|---|---|---|
| | Low | Target | High | | |
| | Hardness | Hardness | Hardness | F23 | F24 |
| Avg Hardness | 4.79 | 8.37 | 11.92 | 0.02 | 0 |
| (kp) | | | | | |
| % Friability | 0.05 | 0.01 | 0.03 | | |
| Disintegration | First: 12 | First: 87 | First: 370 | First: 3.3 | First: 2.0 |
| Time | | | | | |
| in Water | Last: 12 | Last: 117 | Last: 440 | Last: 5.0 | Last: 2.4 |
| (sec) | | | | | |
| Disintegration | First: 12 | First: 58 | First: 316 | First: 2.3 | First: 1.5 |
| Time in pH | | | | | |
| 6.8 Buffer | Last: 22 | Last: 70 | Last: 360 | Last: 3.3 | Last: 1.7 |
| (sec) | | | | | |

Disintegration studies were performed on F24-IRC2 tablets in pH 6.8 buffer and all tablets were found to disintegrate and disperse in under 4 minutes.

Disintegration studies were performed on F23-SC1-EC2 tablets in 0.1N HCl and pH 6.8 buffer. After 2 hrs in 0.1N HCl all the tablets were intact and gave an average acid uptake value of 2.9%. After returning the acid treated tablets to the USP disintegration apparatus and placing in pH 6.8 phosphate buffer media, all tablets disintegrated within approximately 10.5 minutes with no agglomerates remaining.

In a parallel disintegration study on F23-SC1-EC2 tablets wherein the pH 6.8 buffer also contained 2% CTAB it was observed that it took over 1 hour and 5 minutes before all tablets fully disintegrated. No agglomerates were recovered and the tablet contents appeared to be well dispersed. Therefore, CTAB significantly slowed the disintegration of the enteric coated tablets.

Conclusion from Wet Granulation Vs Direct Compression Preparations

Although a direct compression manufacturing process for Compound 1 hydrochloride salt is physically viable, and an uncoated direct compression tablet can be developed that disintegrates very rapidly, the generally unaltered API liberated from coated tablets produced with this process does not adequately disperse in non-acidic media where API solubility is extremely low, even when a non-functional film coat is used. An optimized high shear wet granulation manufacturing process is viable for Compound 1 hydrochloride salt and effectively alters the physical characteristics of the API when combined with other components within the granulation, allowing the API from uncoated or coated tablets produced with this process to adequately disperse in all media. A coating is required to protect the API from exposure to light.

### Example 17: Immediate Release (IR) Tablets

The 60 mg IR tablets prepared according to Example 17 were used in the Phase 1a clinical study described in Example 26.

60 mg IR tablets present as white to off-white, round film-coated tablets. The strength of the active dosage form is 60 mg (calculated as the anhydrous free base form of Compound 1). The composition of the Tablets and a representative batch for Compound 1 IR Tablets is provided in Table 25. The typical batch blend size is 5.5 kg. The theoretical batch size is 13,750 tablets.

TABLE 25
Composition and Batch Formula for Compound 1 IR Tablets

| | | Quantity per | Quantity per |
|---|---|---|---|
| Ingredient | Function | tablet (mg) | Batch (g) |
| Intra-granular | | | |
| Compound 1-HCI (Ex. 2.1) | Active | 67.20 | 924.0 |
| Lactose Monohydrate | Filler/Diluent | 65.47 | 900.3 |
| Microcrystalline Cellulose | Filler/Diluent | 130.33 | 1792.0 |
| Hydroxypropyl Cellulose | Binder | 15.00 | 206.3 |
| Crospovidone | Disintegrant | 12.00 | 165.0 |
| Polysorbate 80 | Wetting Agent | 4.00 | 55.0 |
| Purified Water | Granulation Aid | N/A | N/A |
| Extra-granular | | | |
| Silicified Microcrystalline | Filler/Diluent | 90.00 | 1237.5 |
| Cellulose | | | |
| Crospovidone | Disintegrant | 12.00 | 165.0 |
| Magnesium Stearate | Lubricant | 4.00 | 55.00 |
| Core Tablet | Weight | 400.0 | 5500.0 |
| Opadry II 85F18422 VVhite | Film Coat | 16.00 | 220.0 |
| Purified Water | Solvent | N/A | N/A |
| Film Coated Tablet Weight | | 416.0 | 5720.0 |

NOTE: Water is employed as a granulating agent and to prepare the coating suspension. Water evaporates during drying and the coating process.

The various components used in the formulation may be acquired from standard formulation excipient suppliers, such as: Lactose Monohydrate (Foremost #310); Microcrystalline Cellulose (Avicel PH-102); Hydroxypropyl Cellulose (Klucel EXF Pharm); Crospovidone (Kollidon CL); Silicified Microcrystalline Cellulose (Prosolv SMCC HD 90); Crospovidone (Kollidon CL); Magnesium Stearate (Ligamed MF-2-V).

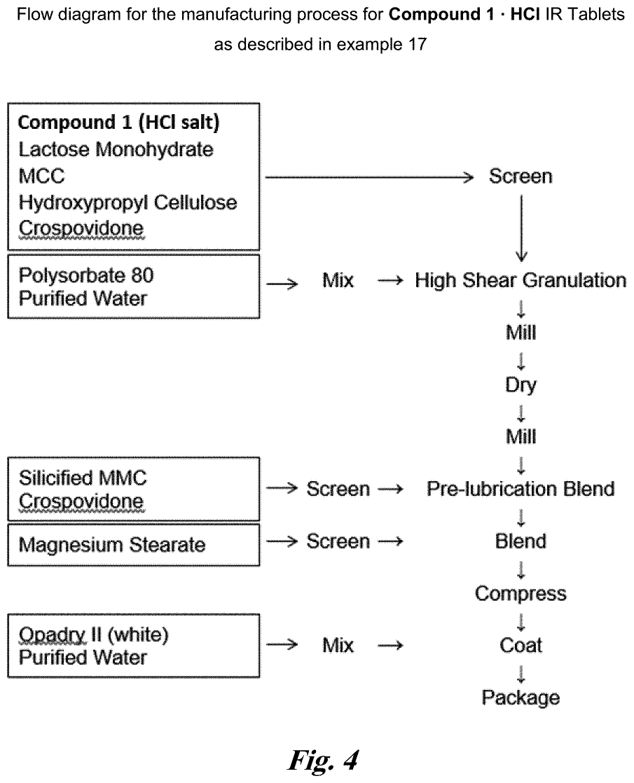

60 mg IR Tablets were prepared via a high-shear wet granulation process. A summary of the process is provided below; a flow diagram for the manufacturing process is provided in FIG. 4.
1. Weigh and dispense materials.
2. Screen or pass the following materials through:
   a. Lactose monohydrate
   b. Compound 1-HCl
   c. Crospovidone (intra-granular)
   d. Hydroxypropyl cellulose
   e. Microcrystalline cellulose
3. Premix the screened materials from Step 2 in a suitably sized high-shear granulator.
4. Dissolve Polysorbate 80 in purified water.
5. Granulate the blend from Step 3 using granulating liquid from Step 4 in suitably sized high-shear granulator.
6. Screen or pass the wet granules through Comil.
7. Dry the wet granules in suitably sized fluid bed dryer.
8. Screen or pass the dry granules through Comil.
9. Transfer the milled granules from Step 8 to a suitably sized blender.
10. Screen or pass the following materials through Comil:
   a. Silicified microcrystalline cellulose
   b. Crospovidone (extra-granular)
11. Blend the material from Step 9 with screened materials from Step 10.
12. Screen magnesium stearate through a suitably sized mesh.
13. Lubricate blend from Step 11 with screened magnesium stearate from Step 12 in a suitably sized blender.
14. Compress the blend from Step 13.
15. Dedust and metal check the tablets.
16. Prepare coating solution with Opadry II (85F18422) White.
17. Coat the tablets with Step 16 in suitably-sized pan.

The in-process controls applied at the compression stage (step 14) include individual tablet weight (370-430 mg); individual tablet hardness (7-11 kilopond) and average weight of 10 tablets (3.800-4.200 g).

### Example 18: DR Tablets

The 60 mg DR tablets prepared according to Example 18 were used in the Phase 1a clinical study described in Example 26.

60 mg DR tablets present as white to off-white, round film-coated tablets. The strength of the active dosage form is 60 mg (calculated as the anhydrous free base of Compound 1). The composition of the Tablets and a representative batch for Compound 1 DR Tablets is provided in Table 26. The typical batch blend size is 5.5 kg. The theoretical batch size is 13,750 tablets.

TABLE 26
Composition and Batch Formula for Compound 1 DR Tablets

| | | Quantity per | Quantity per |
|---|---|---|---|
| Ingredient | Function | Tablet (mg) | Batch (g) |
| Intra-granular | | | |
| Compound 1-HCI (Ex. 2.1) | Active | 67.20 | 924.0 |
| Lactose Monohydrate | Filler/Diluent | 65.47 | 900.3 |
| Microcrystalline Cellulose | Filler/Diluent | 130.33 | 1792.0 |
| Hydroxypropyl Cellulose | Binder | 15.00 | 206.3 |
| Crospovidone | Disintegrant | 12.00 | 165.0 |
| Polysorbate 80 | Wetting Agent | 4.00 | 55.0 |
| Purified Water | Granulation Aid | N/A | N/A |
| Extra-granular | | | |
| Silicified Microcrystalline | Filler/Diluent | 90.00 | 1237.5 |
| Cellulose | | | |
| Crospovidone | Disintegrant | 12.00 | 165.0 |
| Magnesium Stearate | Lubricant | 4.00 | 55.00 |
| Core Tablet Weight | | 400.0 | 5500.0 |
| Opadry 03K19229 Clear | Film Coat | 16.0 | 220.0 |
| Purified Water | Solvent | N/A | N/A |
| Total Sub-Coated Tablet | Weight | 416.0 | 5720.0 |
| Acryl-EZE 93018359 White | Functional Coat | 50.0 | 687.5 |
| Purified Water | Solvent | N/A | N/A |
| Total Enteric Coated | | 466.0 | 6407.5 |
| Tablet Weight | | | |

NOTE: Water is employed as a granulating agent and to prepare the coating suspension. Water evaporates during drying and the coating process.

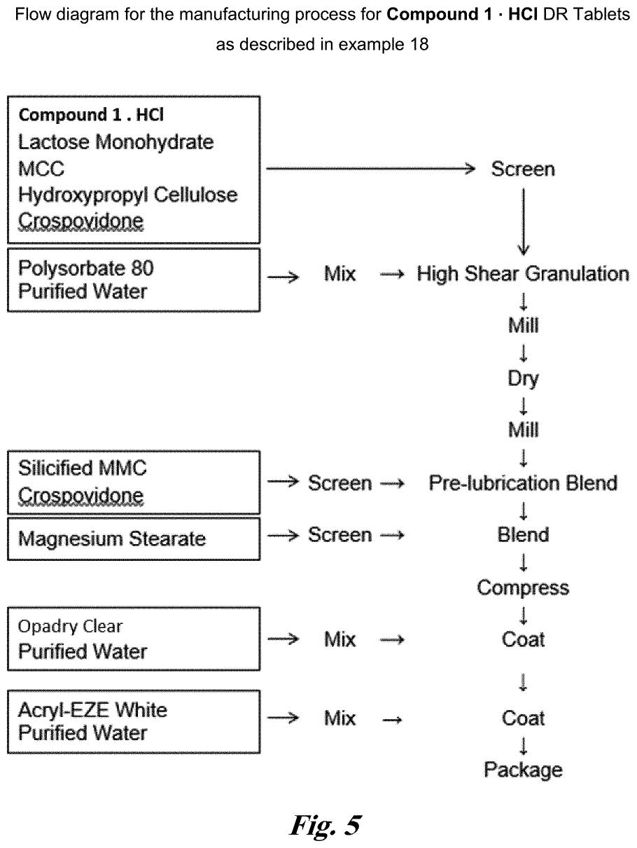

60 mg DR Tablets were prepared via a high-shear wet granulation process. A summary of the process is provided below; a flow diagram for the manufacturing process is provided in FIG. 5.
1. Weigh and dispense materials.
2. Screen or pass the following materials through Comil:
   a. Lactose monohydrate
   b. Compound 1-HCl
   c. Crospovidone (Intra-granular)
   d. Hydroxypropyl cellulose
   e. Microcrystalline cellulose
3. Premix the screened materials from Step 2 in a suitably sized high-shear granulator.
4. Dissolve Polysorbate 80 in purified water.
5. Granulate the blend from Step 3 using granulating liquid from Step 4 in suitably sized high-shear granulator.
6. Screen or pass the wet granules through Comil.
7. Dry the wet granules in suitably sized Fluid Bed Dryer.
8. Screen or pass the dry granules through Comil.
9. Transfer the milled granules from Step 8 into a suitably sized blender.
10. Screen or pass the following materials through Comil:
   a. Silicified microcrystalline cellulose
   b. Crospovidone (Extra-granular)
11. Blend the material from Step 9 with screened materials from Step 10.
12. Screen magnesium stearate through a suitably sized mesh.
13. Lubricate blend from Step 11 with screened magnesium stearate from Step 12 in a suitably sized blender.
14. Compress the blend from Step 13.
15. Dedust and metal check the tablets.
16. Prepare coating solution with Opadry (03K19229) Clear.
17. Coat the tablets with Step 16 in suitably sized pan.
18. Prepare coating solution with Acryl-EZE (93018359) White.
19. Coat the sub-coated tablets with solution from Step 18 in a suitably sized pan.

The in-process controls applied at the compression stage (step 14) include individual tablet weight (370-430 mg); individual tablet hardness (7-11 kilopond) and average weight of 10 tablets (3.800-4.200 g).

### Example 19: Dissolution Rates of IR and DR Tablets

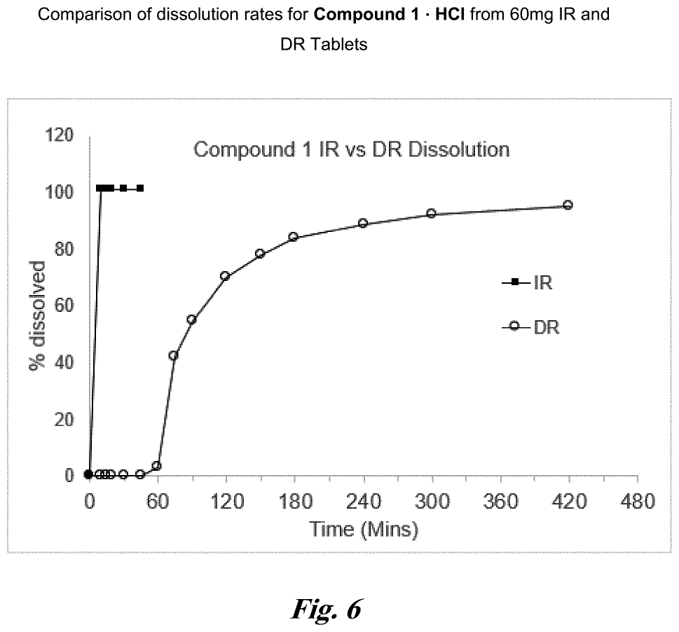

The dissolution rates of the IR and DR Tablets comprising Compound 1⋅HCl, prepared as described in Examples 17 and 18 herein, were determined as detailed in Tables 27 and 28, respectively. The results are presented in Table 29 and shown graphically in FIG. 6.

TABLE 27
Dissolution Parameters for 60 mg IR Tablets

| Dissolution Parameter | Value |
|---|---|
| Apparatus | United States Pharmacopeia Apparatus 2 - Paddles |
| Medium | 0.01N HCI |
| Media Volume | 900 mL |
| Temperature | 37 ± 0.5° C. |
| Rotation Speed | 75 RPM |
| Time Points | 10, 20, 30, and 45 minutes |

TABLE 28
Dissolution Parameters for 60mg DR Tablets

| Dissolution | Value |
|---|---|
| Parameter | |
| Apparatus | United States Pharmacopeia Apparatus 2 - Paddles |
| Medium | Stage 1- 0.01N HCI |
| | Stage 2- 50 mM Phosphate Buffer, pH 6.8 |
| | Stage 3- 50 mM Phosphate Buffer, pH 6.8 with 2% CTAB |
| Media Volume | Stage 1- 900 mL |
| | Stage 2- 300 mL |
| | Stage 3- 900 mL |
| Temperature | 37 ± 0.5° C. |
| Rotation Speed | 75 RPM |
| Time Points | Stage 1- 0.5 hours |
| | Stage 2- 0.5 hours |
| | Stage 3- 0.25, 0.5, 1, 1.5, 2, 3, 4, 6, and 8 hours |

(CTAB: cetyltrimethylammonium bromide)

TABLE 29
Dissolution of IR and DR Tablets

| time (min) | IR | DR |
|---|---|---|
| 0 | 0 | 0 |
| 10 | 101% | 0 |
| 15 | 101% | 0 |
| 20 | 101% | 0 |
| 30 | 101% | 0% |
| 45 | 101% | 0% |
| 60 | | 3% |
| 75 | | 42% |
| 90 | | 55% |
| 120 | | 70% |
| 150 | | 78% |
| 180 | | 84% |
| 240 | | 89% |
| 300 | | 92% |
| 420 | | 95% |
| 540 | | 96% |

### Example 20: Further High Shear Wet Granulation Tablet Formulations—60 mg Tablets

Investigations were carried out to explore alternative high shear wet granulation compositions. Pregelatinized starch (Starch 1500) and spray dried lactose monohydrate (Flowlac 100) were used as diluents. Optimization studies to deliver IR and DR tablets containing 60 mg of parent compound per tablet led to the formulations detailed in Table 30 (formulations F25 and F26).

TABLE 30
60mg IR (F25) and DR (F26) formulations

| Tablet Formulation | | F25 (IR) | | F26 (DR) | |
|---|---|---|---|---|---|
| Ingredients | | mg/tab | %w/w | mg/tab | %w/w |
| Intra-granular | Compound 1 HCI Salt (Ex. 2.1) | 68.6 | 17.1 | 68.6 | 17.1 |
| | Starch 1500 | 20.0 | 5.0 | 20.0 | 5.0 |
| | Lactose Monohydrate Impalpable | 151.4 | 37.9 | 151.4 | 37.9 |
| | Crospovidone | 20.0 | 5.0 | 20.0 | 5.0 |
| | Hydroxy Propyl Cellulose EXF (added wet) | 4.0 | 1.0 | 4.0 | 1.0 |
| | Hydroxy Propyl Cellulose EXF (added dry) | 16.0 | 4.0 | 16.0 | 4.0 |
| | Polysorbate 80 | 4.0 | 1.0 | 4.0 | 1.0 |
| Extra- | Lactose Monohydrate SD | 100.0 | 25.0 | 100.0 | 25.0 |
| granular | Crospovidone | 12.0 | 3.0 | 12.0 | 3.0 |
| | Magnesium Stearate | 4.0 | 1.0 | 4.0 | 1.0 |
| | Core Total: | 400.0 | 100.0 | 400.0 | 100.0 |

| | Film Coat | | — |
|---|---|---|---|
| Ingredients | mg/tab | % w/w | |
| Opadry II White 85F18422 | 16.0 | 4.0 | |
| IR Coated Tablet Total: | 416.0 | 104.0 | |

| | — | Sub-Coat | |
|---|---|---|---|
| Ingredients | | mg/tab | % w/w |
| Opadry Clear 03K19229 | | 16.0 | 4.0 |
| Sub Coated Tablet Total: | | 416.0 | 104.0 |
| | | Enteric Coat | |
| Ingredients | | mg/tab | % w/w |
| Acryl-Eze White 93018359 | | 50.0 | 12.0 |
| DR Tablet Total: | | 466.0 | 112.0 |

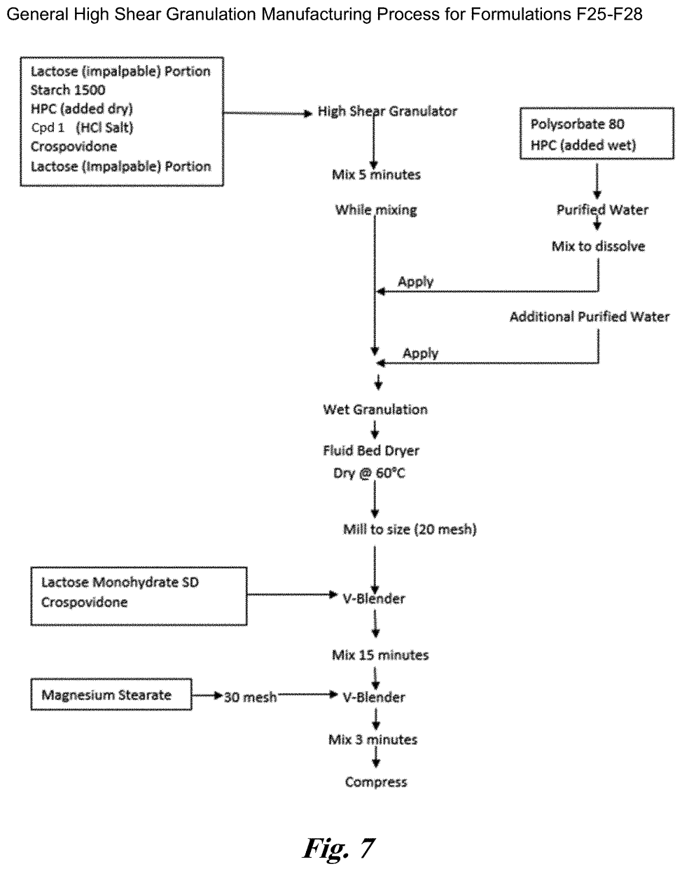

The cores of formulations F25 and F26 were prepared according to a high shear wet granulation process as set out in FIG. 7.

The F25/F26 core composition had the physical characteristics as set out in Table 31.

TABLE 31
F25/F26 blends physical characteristics

| Tests | F25/F26 |
|---|---|
| Bulk Density (g/m1) | 0.595 |
| Tapped Density (g/m1) | 0.735 |
| Carr's Index (%) | 19.1 |
| Hausner Ratio: | 1.24 |
| US Mesh Size (max. | |
| particle size in μm) | % RETAINED |
| 20 (841) | 0.4 |
| 30 (595) | 20.8 |
| 40 (420) | 23.8 |
| 60 (250) | 21.4 |
| 80 (177) | 10.5 |
| 100 (149) | 6.7 |
| 120 (125) | 3.1 |
| 170 (88) | 6.3 |
| 200 (74) | 3.4 |
| 230 (63) | 0.8 |
| Pan | 3.0 |

F25/F26 blended formulations were compressed to produce plain, white to off-white, round, 11 mm Diam., shallow biconvex, bevel edge tablets.

Physical Characteristics of F25 tablets are presented in Table 32. Core tablets were coated using Colorcon's Opadry II 85F18422 White to a weight gain of 4% in an O'Hara Labcoat II fully-perforated coating pan (12″ insert) using the following processing parameters:
Quantity of tablet cores: 800 g/pan load
Conditions:
   Spray nozzle: 1.0 mm
   Gun Angle: 8 o'clock
   Gun Distance: Approx. 4″
   Atomization Air: 15 psi
   Pattern Air: 20 psi
   Air Volume: 130 CFM
   Pan Speed: 20 RPM
   Spray Rate: Approx. 8.4-9.0 g/min
   Inlet Air Temp: 58.5-60.8° C.
   Exhaust Air Temp: 44.6-48.8° C.
   Coating Dispersion: 20% solids, 4% tablet weight gain applied.

TABLE 32
Physical Characteristics of F25 Tablets

| | F25- | F25 - |
|---|---|---|
| | uncoated | coated |
| Avg Hardness (kp) | 7.97 | |
| % Friability | 0.05 | |
| Disintegration Time in Water (min) | First: 1.50 | |
| | Last: 4.17 | |
| Disintegration Time in pH 6.8 Buffer (min) | | First: 1.45 |
| | | Last: 2.00 |

The physical characteristics of the resulting reformulated tablet cores were very good and similar to those of the original high shear formulation containing microcrystalline cellulose (F22, F23, F24). However, the maximum achievable hardness values were slightly lower.

### Example 21: Alternative High Shear Wet Granulation Tablet Formulations—120 mg Tablets

A higher strength 120 mg tablet was developed based on the 60 mg formulations from Example 20, using the same high shear granulation process while keeping the size of the 120 mg strength tablet as small as possible. Table 33 lists the excipient levels used for the IR (F27) and DR (F28) formulations.

TABLE 3
120 mg IR (F27) and DR (F28) formulations

| Tablet Formulation | | F27 (IR) | | F28 (DR) | |
|---|---|---|---|---|---|
| Ingredients | | mg/tab | % w/w | mg/tab | % w/w |
| Intra- | Compound 1 HCl Salt | 137.1 | 27.4 | 137.1 | 27.4 |
| granular | (Ex. 2.1) | | | | |
| | Starch 1500 | 25.0 | 5.0 | 25.0 | 5.0 |
| | Lactose Monohydrate | 147.9 | 29.6 | 147.9 | 29.6 |
| | Impalpable | | | | |
| | Crospovidone | 15.0 | 3.0 | 15.0 | 3.0 |
| | Hydroxy Propyl | 5.0 | 1.0 | 5.0 | 1.0 |
| | Cellulose | | | | |
| | EXF (added wet) | | | | |
| | Hydroxy Propyl | 20.0 | 4.0 | 20.0 | 4.0 |
| | Cellulose | | | | |
| | EXF (added dry) | | | | |
| | Polysorbate 80 | 5.0 | 1.0 | 5.0 | 1.0 |
| Extra- | Lactose | 125.0 | 25.0 | 125.0 | 25.0 |
| | Monohydrate SD | | | | |
| granular | Crospovidone | 15.0 | 3.0 | 15.0 | 3.0 |
| | Magnesium Stearate | 5.0 | 1.0 | 5.0 | 1.0 |
| | Core Total: | 500.0 | 100 | 500.0 | 100 |

| | Film Coat | | — |
|---|---|---|---|
| Ingredients | mg/tab | %w/w | |
| Opadry II White 85F18422 | 20.0 | 4.0 | |
| IR Coated Tablet Total: | 520.0 | 104.0 | |

| | — | Sub-Coat | |
|---|---|---|---|
| Ingredients | | mg/tab | % w/w |
| Opadry Clear 03K19229 | | 20.0 | 4.0 |
| Sub Coated Tablet Total: | | 520.0 | 104.0 |
| | | Enteric Coat | |
| Ingredients | | mg/tab | % w/w |
| Acryl-Eze White 93018359 | | 73.0 | 14.0 |
| DR Tablet Total: | | 593.0 | 114.0 |

The core blends of formulations F27 and F28 were prepared according to a high shear wet granulation process as set out in FIG. 7. The amount of granulating solution containing dissolved HPC (wet portion) and Polysorbate 80 was applied in its entirety during mixing and incremental amounts of water were added to complete the granulation. A granulating solution rate of addition of 55 g/min was used for F27 and a rate of approximately 65 g/min was used for F28.

The F27 and F28 core compositions had the physical characteristics as set out in Table 34.

TABLE 34
F27 & F28 blends physical characteristics

| Tests | F27 | F28 |
|---|---|---|
| Bulk Density (g/ml) | 0.588 | 0.595 |
| Tapped Density (g/ml) | 0.714 | 0.735 |
| Carr′s Index (%) | 17.7 | 19.1 |
| Hausner Ratio: | 1.21 | 1.24 |
| US Mesh Size (max. | | |
| particle size in μm) | % RETAINED | |
| 20 (841) | 0.8 | 0.0 |
| 30 (595) | 20.8 | 13.6 |
| 40 (420) | 16.4 | 13.2 |
| 60 (250) | 24.0 | 20.0 |
| 80 (177) | 13.6 | 14.8 |
| 100 (149) | 8.0 | 10.4 |
| 120 (125) | 3.6 | 5.6 |
| 170 (88) | 7.2 | 10.8 |
| 200 (74) | 2.8 | 5.2 |
| 230 (63) | 0.8 | 1.2 |
| Pan | 1.6 | 3.6 |

F27 and F28 blended formulations were compressed to produce plain, white to off-white, caplet-shaped, biconvex, plain, 17.4 mm×6.7 mm tablets. Physical Characteristics of F27 & F28 tablets are presented in Table 35. Both formulations compressed very well. Weight uniformity was excellent, tablet hardness values remained consistent throughout the runs and friability was very low. The core tablets had disintegration times ranging from under 1 minute to approximately 1 minute and 45 seconds.

TABLE 35
Physical Characteristics of F27 & F28 Tablets

| | F27 | F28 |
|---|---|---|
| Avg Hardness (kp) | 8.81 | 9.42 |
| % Friability | 0.08 | 0.13 |
| Disintegration Time in Water (min) | First: 0.78 | First: 0.92 |
| | Last: 1.08 | Last: 1.75 |

F27 IR tablets were coated using Colorcon's Opadry II 85F18422 White to a weight gain of 4% in an O'Hara Labcoat II fully-perforated coating pan (12″ insert) using the following processing parameters:
Quantity of tablet cores: 800 g/pan load
Conditions:
   Spray nozzle: 1.0 mm
   Gun Angle: 8 o'clock
   Gun Distance: Approx. 4″
   Atomization Air: 15 psi
   Pattern Air: 20 psi
   Air Volume: 130 CFM
   Pan Speed: 20 RPM
   Spray Rate: Approx. 8.8-10.0 g/min
   Inlet Air Temp: 59.0-60.0° C.
   Exhaust Air Temp: 44.6-47.6° C.
   Coating Dispersion: 20% solids, 4% tablet weight gain applied.

Disintegration testing was performed on these F27 IR tablets in pH 6.8 buffer and all tablets were found to disintegrate and disperse from 1 min to 1 min and 13 secs.

F28 DR tablets were prepared by sub-coating core tablets using Colorcon's Opadry Clear 03K19229 to a weight gain of 4% in an O'Hara Labcoat II fully-perforated coating pan (12″ insert) using the following processing parameters:
Quantity of tablet cores: 800 g/pan load
Conditions:
   Spray nozzle: 1.0 mm
   Gun Angle: 8 o'clock
   Gun Distance: Approx. 4″
   Atomization Air: 15 psi
   Pattern Air: 20 psi
   Air Volume: 130 CFM
   Pan Speed: 20 RPM
   Spray Rate: Approx. 8.4-9.1 g/min
   Inlet Air Temp: 57.0-62.0° C.
   Exhaust Air Temp: 44.6-45.3° C.
   Coating Dispersion: 10% solids, 4% tablet weight gain applied.

Sub-coated tablets as prepared above were enteric-coated using Colorcon's Acryl-Eze 93018359 White to a weight gain of 14% in an O'Hara Labcoat II fully-perforated coating pan (12″ insert) using the following processing parameters:
Quantity of tablet cores: 800 g/pan load
Conditions:
   Spray nozzle: 1.0 mm
   Gun Angle: 8 o'clock
   Gun Distance: Approx. 4″
   Atomization Air: 15 psi
   Pattern Air: 20 psi
   Air Volume: 130 CFM
   Pan Speed: 20 RPM
   Spray Rate: Approx. 7.5-9.8 g/min
   Inlet Air Temp: 42.5-50.0° C.
   Exhaust Air Temp: 35.4-37.0° C.
   Coating Dispersion: 20% solids, 14.0% tablet weight gain applied.

Following two hours immersion in 0.1N HCl in the USP disintegration apparatus and calculation of the acid uptake value, the acid treated tablets were returned to the USP disintegration apparatus and placed in pH 6.8 Phosphate Buffer media and observations were made related to their disintegration performance.

The F28 DR tablets showed an average acid uptake of 3.1% and were intact with unchanged appearance after the acid treatment. Upon exposure to pH 6.8 buffer the coating dissolved after approximately 10.5 minutes and all the tablets had fully disintegrated after 11.5 minutes with no agglomerates remaining.

### IR/DR Tablet Stability

### Example 22: Stability Data on Tablet Formulations

IR Tablet formulations prepared by high shear wet granulation were subjected to stability testing under various storage conditions and the purity of Compound 1 by HPLC was assessed initially and then after 1, 3 and 6 months as outlined in Table 36.

TABLE 36
Compound 1 HPLC stability assays for IR tablet formulations

| | | HPLC Assay (total related substances) | | |
|---|---|---|---|---|
| | | F24-IRC2 (A)* | F24-IRC2 (B)* | F25 |
| 2-8° C. | Initial | 98.3 (0.3) | | |
| | 1M | 97.9 (0.3) | | |
| | 3M | 96.9 (0.3) | | |
| | 6M | 97.7 (0.3) | | |
| 25° C./60% | Initial | 98.2 (0.2) | 102.3 (0.12) | 102.0 (0.10) |
| RH | 1M | 98.3 (0.3) | 102.9 (0.18) | 100.2 (0.19) |
| | 3M | 98.1 (0.4) | 101.7 (0.34) | |
| | 6M | 98.4 (0.5) | 98.3 (0.38) | |
| 30° C./65% | Initial | 98.2 (0.2) | | 102.0 (0.10) |
| RH | 1M | — | | |
| | 3M | — | | |
| | 6M | 97.9 (0.7) | | |
| 40° C./75% | Initial | 98.2 (0.2) | 102.3 (0.12) | 102.0 (0.10) |
| RH | 1M | 98.0 (0.7) | 102.2 (0.53) | 99.8 (0.32) |
| | 3M | 96.5 (1.4) | 100.5 (1.16) | |
| | 6M | 96.0 (2.3) | 96.7 (1.88) | |

*assessed at different test sites

DR Tablet formulations prepared by high shear wet granulation (F23-SC1-EC2) were also subjected to stability testing under various storage conditions and the purity of Compound 1 by HPLC was assessed initially and then after 1, 3 and 6 months as outlined in Table 37.

The F16-SC1-EC2 tablets had been prepared by direct compression, without water addition to the processing. Despite the F23-SC1-EC2 tablets having had water added during the wet granulation processing, this did not result in increased degradation being seen in the six-month stability study; in fact slightly more degradation was observed for the direct compression tablets than was seen with the wet granulation tablets. As can be seen from Table 37, after 6 months at 25° C./60% RH and at 40° C./75% RH, the levels of total related impurities seen with the direct compression tablets were 0.91% and 2.70% respectively. None of the IR or DR tablets prepared by high shear wet granulation had such high levels of related impurities after 6 months under comparable conditions.

It would therefore appear that, surprisingly, the chemical stability of Compound 1 on storage is slightly better when formulated in wet granulation compositions according to the present invention, as opposed to comparable compositions prepared by direct compression.

TABLE 37
Compound 1 HPLC stability assays for DR tablet formulations

| | | HPLC Assay (total related substances) | | |
|---|---|---|---|---|
| | | F23-SC1- | F23-SC1- | F16-SC1-EC2 |
| | | EC2 (A)* | EC2 (B)* | (direct compression) |
| 2-8° C. | Initial | 97.6 (0.3) | | |
| | 1M | 97.1 (0.3) | | |
| | 3M | 96.7 (0.3) | | |
| | 6M | 97.7 (0.4) | | |
| 25° C./ | Initial | 98.2 (0.2) | 101.1 (0.11) | — |
| 60% RH | 1M | 97.6 (0.3) | 102.6 (0.18) | — |
| | 3M | 97.5 (0.4) | 100.6 (0.31) | — |
| | 6M | 97.6 (0.6^{a}) | 97.8 (0.46) | 98.9 (0.91^{a}) |
| 30° C./ | Initial | 98.2 (0.2) | | |
| 65% RH | 1M | — | | |
| | 3M | — | | |
| | 6M | 97.5 (0.9) | | |
| 40° C./ | Initial | 98.2 (0.2) | 101.1 (0.11) | — |
| 75% RH | 1M | 97.2 (0.8) | 100.8 (0.44) | — |
| | 3M | 95.9 (1.6) | 99.4 (1.23) | — |
| | 6M | 95.4 (2.6^{b}) | 95.6 (1.86) | 94.4 (2.70^{b}) |

*assessed at different test sites
^{a}When tested at same test site, F23-SC1-EC2 and F16-SC1-EC2 had 0.16% and 0.6% respectively of a related substance with RRT of 0.43-0.44 by HPLC c.f. Compound 1
^{b}When tested at same test site, F23-SC1-EC2 and F16-SC1-EC2 had 0.20% and 0.72% respectively of a related substance with RRT of 0.43-0.44 by HPLC c.f. Compound 1

### Clinical Trials

### Example 23: A Phase 1a Randomized, Double-Blind, Placebo-Controlled, Single Ascending Dose Clinical Study to Assess the Safety, Tolerability, Pharmacokinetics, and Pharmacodynamic Effects of Compound 1 (Solution) in Healthy Male Volunteers

Compound 1 was administered as the HCl-salt (prepared analogously to Example 2.1). Compound 1 was administered orally in a pH adjusted water solution (pH=2.5), also containing hydroxypropyl-β-cyclodextrin (HPβCD) as a solubilizing agent, as detailed in Table 38:

TABLE 38
Compound 1 Oral solution

| Component | Function | Amount |
|---|---|---|
| Compound 1-HCl | Active | 0.4-4.8 mg/mL |
| HPβCD | Solubilizing agent | 100 mg/mL |
| Hydrochloric acid, 1N | Adjust pH | pH 2.5 |
| Sterile water | Vehicle, irrigation | 150 mL |

Placebo dosing solutions were identical to active, except without Compound 1. Subjects received a single oral solution of 50 mL or 100 mL; all subjects received a total volume of 150 mL of liquid. Since HPβCD concentration remained constant, subjects in Cohort 5 received twice the amount of HPβCD relative to subjects in Cohorts 1 to 4. Dosing, including consumption of rinse water, was completed within 3 minutes, administered in the morning after an overnight fast, and maintained for up to 4 hours after dosing.

The primary objective of the study was to assess the safety and tolerability of ascending dose levels of Compound 1 after single oral dose administration. The safety endpoints included AE incidence, clinically significant changes in vital signs, ECG parameters, clinical laboratory tests and physical examination.

The secondary objective was to characterize the single-dose pharmacokinetic (PK) parameters of Compound 1 after ascending doses. The following PK parameters were determined from the plasma concentrations of Compound 1 using standard methods of non-compartmental analysis:
Cₘₐₓ maximum plasma drug concentration
Tₘₐₓ time to maximum plasma drug concentration
t_{1/2} half-life
AUCₗₐₛₜ area under the concentration-time curve from time 0 to last quantifiable concentration
AUC₁₂ area under the concentration-time curve from time 0 to 12 hours
AUC₀₋ᵢₙₜ area under the concentration-time curve extrapolated to infinity
λ_{z} elimination rate constant
CL/F apparent clearance
V/F apparent volume of distribution

40 healthy male subjects were randomized into 5 cohorts of 8 subjects each. Subjects within each cohort were assigned to randomized treatment; 6 assigned to Compound 1 and 2 assigned to placebo. Subjects were infection free (including HIV, hepatitis B or hepatitis C), no history of chronic disease or cancer, alcohol dependence, drug addiction or nicotine use. Each cohort consisted of white, black or African American, and Asian male subjects although the majority were white (47.5%). Subject demographics are summarized in Table 39.

TABLE 39
Study Subject Demographics

| Demographic | Total (N = 40) |
|---|---|
| Age (years)^{a} | |
| Mean (SD) | 36.2 (8.83) |
| Median | 37.0 |
| Range | 20-48 |
| Race, n (%) | |
| White | 19 (47.5) |
| Black or African American | 11 (27.5) |
| Asian | 8 (20.0) |
| Other | 2 (5.0) |
| Body Mass Index (kg/m²) | |
| Mean (SD) | 25.61 (2.693) |
| Median | 25.75 |
| Range | 20.2-30.3 |

Cohorts were studied sequentially starting with the lowest dose. The decision to enroll the cohort at the next dose level was reviewed by a safety monitoring team and based on the safety and PK data from previous dose cohorts. Progression to the next higher dose only occurred if the previous dose level was deemed safe and well tolerated. The treatments administered are summarized in Table 40.

TABLE 40
Compound 1 and Placebo Administration

| | Compound | Compound | Vol | Vol | Tot |
|---|---|---|---|---|---|
| | 1 | 1 | Dosing | Rinsing | Vol |
| Cohort | Dose | Conc | Solution ^{a} | Water ^{b} | Administered |
| 1 | 20 mg | 0.4 mg/mL | 50 mL | 100 mL | 150 mL |
| 2 | 60 mg | 1.2 mg/mL | 50 mL | 100 mL | 150 mL |
| 3 | 120 mg | 2.4 mg/mL | 50 mL | 100 mL | 150 mL |
| 4 | 240 mg | 4.8 mg/mL | 50 mL | 100 mL | 150 mL |
| 5 | 240 mg | 2.4 mg/mL | 100 mL | 50 mL | 150 mL |

^{a}For Cohorts 1 to 4, one dosing container with 50 mL of dosing solution was administered. For Cohort 5, two dosing containers, each with 50 mL of dosing solution, were administered.
^{b}For Cohorts 1 to 4, the dosing container was rinsed with a total of 100 mL of water. For Cohort 5, each dosing container was rinsed with 25 mL of water.

All subjects underwent a complete physical examination and their medical history and demographic information collected. The body systems evaluated included general appearance, skin, lymphatic, head and neck, EENT (eyes, ears, nose, and throat), chest and lungs, cardiovascular, abdomen, extremities, musculoskeletal and neuromuscular. Vital signs were measured. Blood pressure, pulse, respiratory rate and oral temperature; were taken. Blood and urine samples were collected at screening, pre-baseline, baseline, during the treatment period, and Day 8 follow-up visit; the following clinical laboratory evaluations were conducted.

Clinical Laboratory Assessments included hematology (complete blood count (CBC), hemoglobin, hematocrit, red blood cell (RBC), mean corpuscular cell volume, mean corpuscular hemoglobin, mean corpuscular hemoglobin concentration, red cell distribution width, mean platelet volume, white blood cell count with differential (neutrophils, immature granulocytes, lymphocytes, monocytes, eosinophils, basophils), platelets, and automated reticulocyte count); chemistry (sodium, potassium, bicarbonate, chloride, calcium, phosphorus, fasting glucose, creatinine, blood urea nitrogen, creatinine phosphokinase, uric acid, albumin, total protein, total bilirubin, alkaline phosphatase, ALT, AST, lactate dehydrogenase, total cholesterol and triglycerides); and urinalysis (bilirubin, blood glucose, ketones, pH, protein, specific gravity and microscopic examination).

Blood samples were collected at baseline, day 1 (0.5, 1, 2, 3, 4, 6, 8, 12, 16, 24 hours post dose), day 2 (30, 36 and 48 hours post dose) and day 8 follow-up visit. Analysis of plasma samples was performed using a validated Liquid Chromatography—Mass Spectrometry and Liquid Chromatography—Tandem Mass Spectrometry (LC-MS/MS) method.

40 subjects received at least 1 dose. Ten subjects who received placebo across all cohorts were excluded from the PK analyses. Thus, only 30 subjects (75%) comprised the PK Population.

Four subjects from Cohort 4 experienced emesis within 1.5 hours post-dose, which likely affected PK results. Therefore, PK summaries were created with and without the subjects who experienced emesis. The 240 mg dose level was repeated in Cohort 5 at a modified concentration−twice the volume (100 mL instead of 50 mL) with half the concentration used in Cohort 4 (2.4 mg/mL instead of 4.8 mg/mL). The dose for Cohort 5 contained twice the amount of HPβCD relative to Cohort 4.

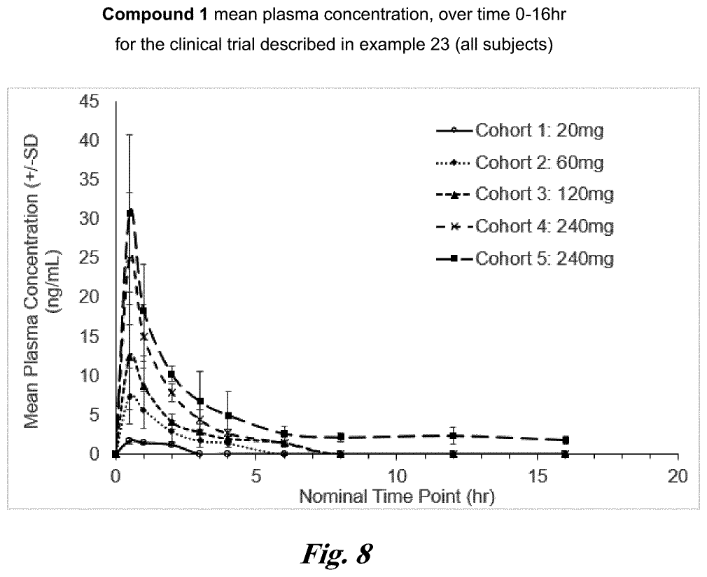

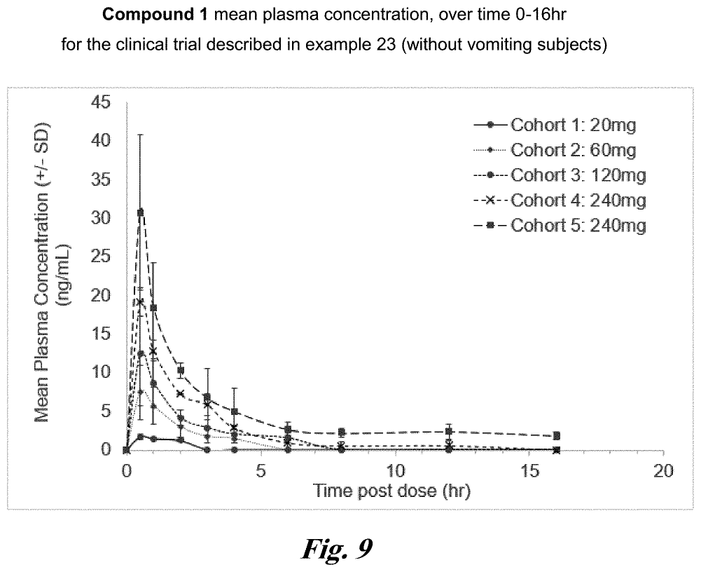

PK profiles (Compound 1 mean plasma concentration, over time 0-16 hours), are presented in Table 41, and FIGS. 8 and 9 for all subjects and subjects who did not experience vomiting (ie the 4 subjects in Cohort 4). The profiles show Compound 1 was rapidly absorbed, with Tₘₐₓ of less than 1 hour. The peak concentrations, Cₘₐₓ and AUC parameters increased with increasing dose and were higher for drug administered in a larger volume (i.e., Cohort 5, 240 mg in 100 mL vehicle) compared with the other cohorts. Elimination parameters (t_{1/2} CL/F) suggested rapid clearance from plasma and relatively large volume of distribution.

TABLE 41
Summary of Key Mean Pharmacokinetic parameters (SD)

| | | Cₘₐₓ | Tₘₐₓ | AUCₗₐₛₜ |
|---|---|---|---|---|
| Cohort | Dose | (ng/mL) | (h) | (h · ng/mL) |
| Cohort 1 | 20 mg | 2.06 (0.75) | 0.5 | 10.70 (21.87) |
| Cohort 2 | 60 mg | 7.71 (3.52) | 0.5 | 14.15 (8.87) |
| Cohort 3 | 120 mg | 12.42 (7.48) | 0.5 | 32.32 (23.82) |
| Cohort 4 | 240 mg | 25.02 (9.20) | 0.5 | 41.29 (9.01) |
| Cohort 5 | 240 mg | 30.73 (11.02) | 0.5 | 79.51 (28.85) |

Hummel Power analysis revealed linear dose proportionality for Cₘₐₓ, both for all subjects and for the subset excluding the four cohort 4 emesis subjects. The slope value was close to 1 and the 90% confidence interval (Cl) for the slope was contained within the critical interval (ie, 0.72-1.28). AUCₗₐₛₜ values were slightly above linearity with a slope of 1.167 for all subjects and 1.231 for subjects without emesis. The upper bound of the 90% Cl for the slope in both cases was above the critical range.

Primary Endpoint Conclusion—Safety and Tolerability: All doses evaluated (20-240 mg) in 50 mL or 100 mL vehicle were determined to be safe and well tolerated. No deaths or SAEs occurred and all of the TEAEs reported were mild in severity. No clinically significant laboratory abnormalities occurred.

Secondary Endpoint Conclusion—Pharmacokinetics: Following a single oral solution formulation dose of Compound 1, the plasma concentration of Compound 1 increased rapidly (Tₘₐₓ of 30 minutes), i.e. Compound 1 was rapid absorbed. Cₘₐₓ and AUC increased in close to dose proportional manner over the entire dose range of Compound 1.

### Example 24: A Phase 1a Randomized, Double-Blind, Placebo-Controlled, Multiple Ascending Dose Clinical Study to Assess the Safety, Tolerability, Pharmacokinetics, and Pharmacodynamic Effects of Compound 1 (Solution) in Healthy Male and Female Volunteers

Compound 1 was administered as the HCl-salt (prepared analogously to Example 2.1). Compound 1 was administered orally in 100 mL of a 10% hydroxypropyl-beta-cyclodextrin (HPβCD) solution, once daily for 8 days. Placebo consisted of a HPβCD solution administered at a volume and dose schedule corresponding with that of Compound 1. See Table 42.

TABLE 42
Planned doses and dose levels

| | Subjects | Placebo | Total | | Vol* | Compound 1 |
|---|---|---|---|---|---|---|
| Cohort | (n) | (n) | (n) | Dose | administered | Conc |
| 1 | 6 | 2 | 8 | 60 mg | 100 mL | 0.6M |
| 2 | 6 | 2 | 8 | 120 mg | 100 mL | 1.2M |
| 3 | 6 | 2 | 8 | 240 mg | 100 mL | 2.4M |
| 4 | 6 | 2 | 8 | 120 mg | 100 mL | 1.2M |
| 5 | 6 | 2 | 8 | 60 mg | 100 mL | 0.6M |

*Vol of 10% HPβCD solution

The primary objective of the study was to assess the safety and tolerability of ascending doses of Compound 1 after multiple oral doses administration. The safety endpoints included TEAE incidence and severity, clinically significant changes in vital signs, ECG parameters, clinical laboratory tests and physical examination.

The secondary objective was to characterize the multiple dose pharmacokinetic (PK) parameters of Compound 1, after ascending doses. Secondary endpoints included determination of PK parameters in plasma and urine, fecal excretion and assessment of colonic biopsy samples.

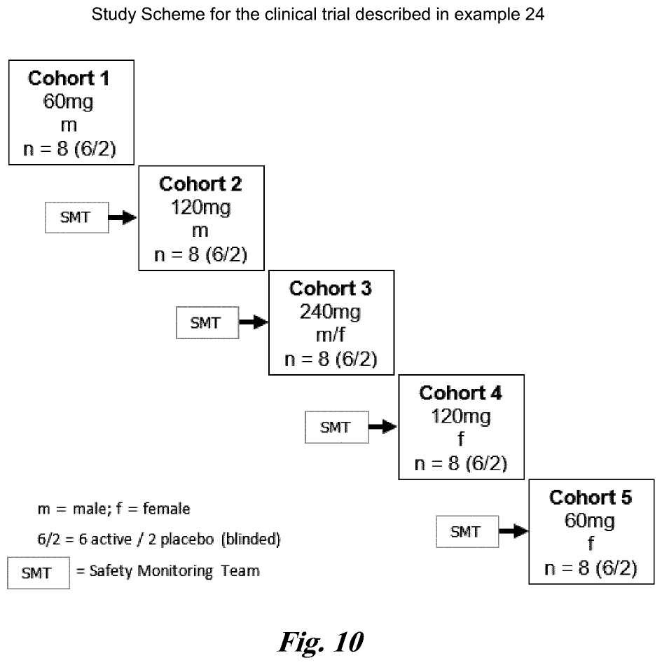

40 healthy subjects were randomized into 5 dose cohorts of 8 subjects each. Subjects received Compound 1 (n=6) or placebo (n=2), administered once daily for 8 days. The first 3 dose cohorts were in an ascending-dose design. Prior to each dose escalation, a safety monitoring team determined if dose escalation was appropriate. The planned dose levels are given in table 42 The planned study scheme is presented as a flow chart in FIG. 10.

Thus, the study planned to enroll up to 40 subjects (up to 30 assigned to Compound 1 and up to 10 assigned to placebo), with allowance for replacement subjects. The study actually enrolled 42 subjects, consisting of 32 subjects treated with Compound 1 (including 2 replacement subjects) and 10 subjects treated with placebo. Overall, 15 male and 17 female subjects were treated with Compound 1, and 5 male and 5 female subjects were treated with placebo.

Cohorts 1 and 2 evaluated male subjects exclusively; Cohort 3 (240 mg) evaluated both male and female subjects. Upon completion of Cohort 3, Cohorts 4 and 5 were added to evaluate the 120 mg and 60 mg doses in females. As with Cohorts 1-3, Cohorts 4 and 5 were randomized to receive Compound 1 (n=6) or placebo (n=2).

Subjects were healthy males or females, age 18 to 55, with a Body Mass Index (BMI) between 18 to 33 kg/m². Subjects were infection free (including HIV, hepatitis B or hepatitis C), no history of chronic disease or cancer, alcohol dependence, drug addiction or nicotine use, and did not display any significant colorectal symptoms or findings. The actual subject demographics and baseline characteristics are summarized in table 43.

TABLE 43
Subject Demographics

| Cohort: | Placebo | | 1 | | 2 | | 3 | | 4 | | 5 | | All | |
|---|---|---|---|---|---|---|---|---|---|---|---|---|---|---|
| n: | 10 | | 6 | | 6 | | 8 | | 6 | | 6 | | 42 | |
| Age Mean (SD) | 49.6 | (6.43) | 42.0 | (9.51) | 39.2 | | 47.5 | (8.37) | 53.3 | (1.97) | 52.8 | (2.71) | 47.6 | (8.33) |
| Age Range | 32-54 | | 27-53 | | 27-50 | | 28-54 | | 50-55 | | 49-55 | | 27-55 | |
| Gender n (%) - M | 5 | (50.0) | 6 | (100.0) | 6 | (100.0) | 3 | (37.5) | 0 | | 0 | | 20 | (47.6) |
| Gender n (%) - F | 5 | (50.0) | 0 | | 0 | | 5 | (62.5) | 6 | (100.0) | 6 | (100.0) | 22 | (52.4) |
| Race, n (%) - W | 7 | (70.0) | 4 | (66.7) | 3 | (50.0) | 7 | (87.5) | 5 | (83.3) | 4 | (66.7) | 30 | (71.4) |

| | | | | | | | | | | | | |
|---|---|---|---|---|---|---|---|---|---|---|---|---|
| Race, n (%) - B | 2 | (20.0) | 2 | (33.3) | 1 | (16.7) | 0 | 0 | 1 | (16.7) | 6 | (14.3) |

| | | | | | | | | | | | | |
|---|---|---|---|---|---|---|---|---|---|---|---|---|
| Race, n (%) - A | 1 | (10) | 0 | 2 | (33.3) | 1 | (12.5) | 1 | (16.7) | 0 | 5 | (11.9) |

| | | | | | | | | | |
|---|---|---|---|---|---|---|---|---|---|
| Race, n (%) - O | 0 | 0 | 0 | 0 | 0 | 1 | (16.7) | 1 | (2.4) |

| | | | | | | | |
|---|---|---|---|---|---|---|---|
| BMI (kg/m²) | 25.2 | 26.16 | 25.74 | 24.63 | 25.71 | 25.19 | 25.3 |
| Mean (SD) | (3.62) | (1.02) | (1.85) | (1.92) | (3.56) | (4.25) | (1.96) |
| BMI Range | 20-33 | 25-28 | 23-28 | 21-27 | 22-31 | 19-31 | 19-33 |

W = white; B = Black or African American; A = Asian; O = Other

The study included the following periods:

Screening (up to 30 days prior to dosing)

Treatment Period (Days 1-8)

Follow-Up telephone call 1 week after last dose administration (Day 15).

Subjects fasted for a minimum of 8 hours prior to obtaining blood samples. During the treatment period, subjects fasted from at least 8 hours prior to dosing in the morning. After dosing, subjects are served a standard breakfast after the 2-hour blood sample has been collected (on Day 1 and 7), a standard lunch approximately 4 hours post-dose, a dinner approximately 10 hours post-dose, and a light snack 12-14 hours post-dose.

Safety and tolerability were assessed by review of vital signs, laboratory results (serum chemistry, hematology and urinalysis), ECG, physical examination findings, AEs, and PK assessment following the Day 1 and Day 7 doses. All cohorts indicated Compound 1 was generally well tolerated at doses up to 120 mg. The 240 mg dose was less well tolerated, primarily due to episodes of dizziness and nausea. One event of vomiting led to early trial discontinuation in the 240 mg dose group.

Blood samples were collected at the following time points relative to dose administration on Day 1 and Day 7: pre-dose; 0.5, 1, 2, 3, 4, 6, 8, 12, 16 and 24 hours post dose. Analysis and concentration determinations were performed using a liquid chromatography-mass spectrometry and liquid chromatography-tandem mass spectrometry (LC/MS/MS) method.

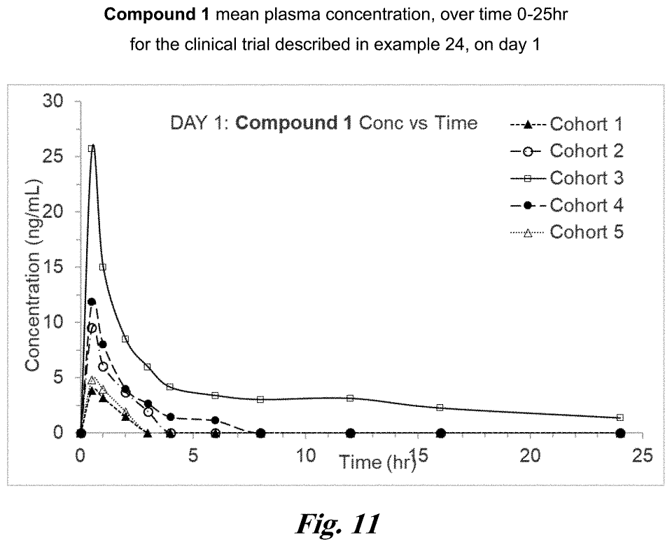

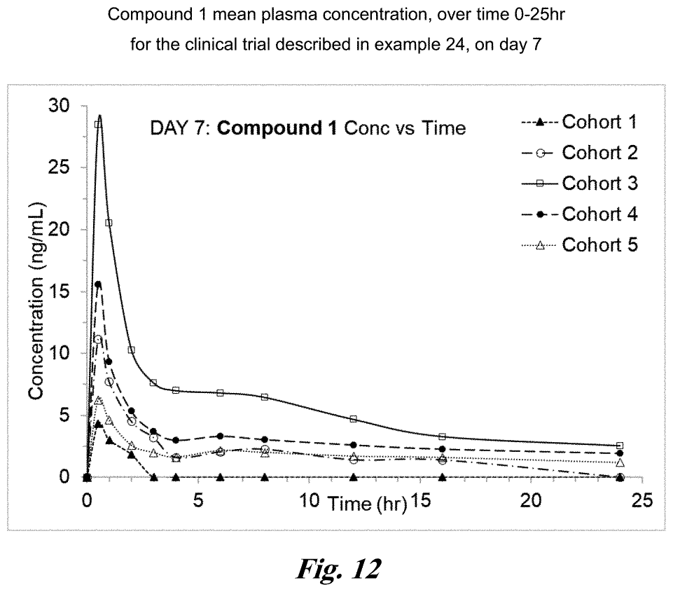

Mean (+/−SD) plasma concentrations of Compound 1, at the noted timepoints, on day 1 and day 7 are presented in Tables 44 and 45. FIGS. 11 and 12 show the mean plasma concentration-time profiles of Compound 1 on day 1 (single dose) and day 7 (multiple dose) respectively.

TABLE 44
Compound 1 concentration (ng/mL) on Day 1
Conc (SD)

| DAY 1 | Cohort 1 | Cohort 2 | Cohort 3 | Cohort 4 | Cohort 5 |
|---|---|---|---|---|---|
| Timepoint | (N = 6) | (N = 6) | (N = 8) | (N = 6) | (N = 6) |
| Pre-dose | 0 | 0 | 0 | 0 | 0 |
| Hour 0.5 | 3.875 (0.58) | 9.542 (1.66) | 25.738 (12.84) | 11.897 (3.97) | 4.828 (0.78) |
| Hour 1 | 3.160 (1.09) | 6.077 (1.06) | 14.975 (6.28) | 8.027 (3.23) | 3.942 (0.78) |
| Hour 2 | 1.472 (0.84) | 3.708 (0.59) | 8.510 (4.46) | 4.005 (1.88) | 1.932 (0.66) |
| Hour 3 | 0 | 1.948 (0.47) | 5.966 (3.43) | 2.680 (1.45) | 0 |
| Hour 4 | 0 | 0 | 4.125 (2.77) | 1.477 (1.08) | 0 |
| Hour 6 | 0 | 0 | 3.390 (2.62) | 1.140 (1.57) | 0 |
| Hour 8 | 0 | 0 | 3.024 (2.17) | 0 | 0 |
| Hour 12 | 0 | 0 | 3.131 (3.72) | 0 | 0 |
| Hour 16 | 0 | 0 | 2.255 (1.99) | 0 | 0 |
| Hour 24 | 0 | 0 | 1.351 (1.26) | 0 | 0 |

TABLE 45
Compound 1 concentration (ng/mL) on Day 7
Conc (SD)

| DAY 7 | Cohort 1 | Cohort 2 | Cohort 3 | Cohort 4 | Cohort 5 |
|---|---|---|---|---|---|
| Timepoint | (N = 6) | (N = 6) | (N = 8) | (N = 6) | (N = 6) |
| Pre-dose | 0 | 0 | 3.016 (1.79) | 1.912 (1.75) | 0 |
| Hour 0.5 | 4.325 (1.30) | 11.115 (3.21) | 28.460 (14.83) | 15.562 (7.47) | 6.237 (2.21) |
| Hour 1 | 3.023 (0.51) | 7.703 (1.02) | 20.520 (8.48) | 9.342 (3.67) | 4.600 (1.25) |
| Hour 2 | 1.855 (0.53) | 4.502 (0.98) | 10.280 (3.33) | 5.318 (2.25) | 2.570 (1.02) |
| Hour 3 | 0 | 3.190 (1.22) | 7.610 (3.88) | 3.688 (1.67) | 1.990 (1.63) |
| Hour 4 | 0 | 1.598 (0.99) | 6.988 (5.37) | 2.975 (1.90) | 1.618 (1.43) |
| Hour 6 | 0 | 2.045 (1.77) | 6.802 (6.36) | 3.298 (1.68) | 2.148 (2.21) |
| Hour 8 | 0 | 2.258 (1.83) | 6.444 (3.94) | 3.030 (1.54) | 2.002 (1.82) |
| Hour 12 | 0 | 1.405 (1.21) | 4.662 (1.91) | 2.592 (1.42) | 1.702 (1.65) |
| Hour 16 | 0 | 1.393 (1.32) | 3.274 (1.58) | 2.260 (1.63) | 1.592 (1.34) |
| Hour 24 | 0 | 0 | 2.534 (1.62) | 1.917 (1.66) | 1.180 (1.06) |

Mean PK parameters for Day 1 are presented in Table 46; mean PK parameters for Day 7 are presented in Table 47.

TABLE 46
Mean (SD) PK Parameters Day 1

| | | Cₘₐₓ | Tₘₐₓ | AUC_{(0-τ)} |
|---|---|---|---|---|
| Cohort | n | (ng/mL) | (h)^{a} | (ng · h/mL) |
| 1 | 6 | 4.11 (0.57) | 0.5 (0.5-1.0) | 5.32^{b} (2.23) |
| 2 | 6 | 9.54 (1.66) | 0.5 (0.5-1.0) | 27.95 (11.01) |
| 3 | 8^{C} | 25.74 (12.84) | 0.5 (0.5-1.0) | 91.54 (52.53) |
| 4 | 6 | 12.00 (4.02) | 0.5 (0.5-1.0) | 38.12 (7.94) |
| 5 | 6 | 5.06 (0.71) | 0.5 (0.5-1.0) | 28.70 (19.98) |

TABLE 47
Mean (SD) PK Parameters Day 7

| Cohort | n | Cₘₐₓ (ng/mL) | | Tₘₐₓ (h)^{a} | | AUC₍₀₋ₜ₎ (ng · h/mL) | | AR_{Cmax} | | AR_{AUC} | |
|---|---|---|---|---|---|---|---|---|---|---|---|
| 1 | 6 | 4.33 | (1.30) | 0.5 | (0.5-0.5) | 17.0 | (8.91) | 1.0 | (0.20) | 2.1 | (1.05) |
| 2 | 6 | 11.39 | (2.72) | 0.5 | (0.5-0.5) | 48.2 | (24.82) | 1.2 | (0.14) | 1.8 | (0.82) |
| 3 | 5 | 28.76 | (14.40) | 0.5 | (0.5-0.5) | 138.7 | (62.16) | 1.0 | (0.31) | 1.4 | (0.24) |
| 4 | 6 | 15.56 | (7.47) | 0.5 | (0.5-0.5) | 75.8 | (37.40) | 1.3 | (0.20) | 2.3 | (1.66) |
| 5 | 6 | 6.31 | (2.07) | 0.5 | (0.5-0.5) | 53.2 | (30.18) | 1.3 | (0.43) | 3.0 | (2.69) |

^{a}Tₘₐₓ is presented as median (range).
^{b}AUCₗₐₛₜ is presented as AUC₍₀₋ₜ₎ could not be estimated for several subjects.
^{c}Two additional subjects enrolled in this cohort as replacement for subjects who prematurely
withdrew
SD: standard deviation
Cₘₐₓ: maximum plasma drug concentration
Tₘₐₓ: time to reach Cmax
AUCₗₐₛₜ: area under concentration-time curve from time zero to last quantifiable concentration
AR_{AUC}: accumulation ratios of AUC0
ARCₘₐₓ: accumulation ratio of Cₘₐₓ

Fecal samples were collected, when possible, over 24 hours (0-24 hours) post administration on Day 1 and Day 7; the absolute amount of Compound 1 measured (mg) is shown Table 48.

TABLE 48
Mean observed amount of Compound 1 (mg) in fecal samples on Days 1 and 7

| | | Cohort 1 | | Cohort 2 | | Cohort 3 | | Cohort 4 | | Cohort 5 | |
|---|---|---|---|---|---|---|---|---|---|---|---|
| | | (N = 6) | | (N = 6) | | (N = 8) | | (N = 6) | | (N = 6) | |
| DAY 1 | N | 3 | | 4 | | 4 | | 5 | | 2 | |
| | Mean (SD) | 11.8 | (7.4) | 5.9 | (9.5) | 46.5 | (29.0) | 14.4 | (12.9) | 13.0 | (5.3) |
| DAY 7 | N | 3 | | 4 | | 5 | | 6 | | 4 | |
| | Mean (SD) | 22.0 | (27.7) | 37.5 | (23.3) | 125.3 | (59.9) | 33.9 | (20.7) | 11.6 | (3.7) |

Following ascending doses, mean fecal concentrations of Compound 1 increased in a dose-dependent manner and ranged across cohorts from approximately 40-225 μg/g on Day 1, and 95-506 μg/g on Day 7. Mean recovery of Compound 1 in feces across cohorts ranged from approximately 5-22% on Day 1 and 19-52% on Day 7.

Urine samples were collected at the following time points relative to dose administration on Day 1 and Day 7: pre-dose; 0-4, 4-8, and 8-24 hours post dose.

Colonic mucosal biopsy samples were acquired. Compound 1 tissue levels are presented in Table 49 and shown to increase in a dose-dependent manner.

TABLE 49
Median (Range) Concentrations of Compound 1 in Colon Tissue

| | Dose | | Compound 1 |
|---|---|---|---|
| Cohort | (mg) | N | Concentration (ng/g) |
| 1 | 60 | 6 | NA |
| 2 | 120 | 6 | 10.75 (0-1150) |
| 3 | 240 | 5 | 23.50 (0-301) |
| 4 | 120 | 6 | 127.47 (0-3330) |
| 5 | 60 | 6 | 102.30 (0-1360) |

NA: not available as 5 of 6 subjects had concentrations of Compound 1 below limit of quantitation.
N = 2, 1, 1, 1, and 2 subjects in Cohort 2, 3, 4, 5 and 6, respectively, for whom Compound 1concentrations were below the limit of quantitation

Colonic mucosal biopsy samples were also assessed for hypoxia inducible factor (HIF)-inducible gene expression. For Tier 1 association tests, two significant gene were detected (HMOX1, CA9). For Tier 2 analysis, 8 significant genes were detected (NDRG1, LCN2, BNIP3, TFF3, AKR1C3, BIRC3, TMEM45A and HILPDA). These genes are associated with HIF-1α and/or hepcidin antimicrobial peptide (HAMP) suggesting the prolyl hydroxylase domain (PHD) target is engaged by Compound 1.

Manual assessment of the proportion of HIF-1α positive cells was conducted in 10 regions of interest, selected in whole tissue sections. (Note, the study was amended to add this immunohistochemistry analysis; thus data is only available for cohorts 4 and 5 (120 mg and 60 mg), cohorts 1-3 lacked this type of biopsy).

Table 50 presents the proportions of HIF-1α positive cells evaluated at baseline and Day 8, and the change from baseline to Day 8 of the proportion of HIF-1α positive cells. The mean proportion of HIF-1α positive cells ranged from 1% to 80% across all experimental groups. The proportion of HIF-1α positive cells increased from baseline to Day 8 across all groups and a numerically higher increase was seen for the combined group (“Pooled”) (35.2+28.8 [mean±SD]) compared to the placebo group (13.7±20.9 [mean±SD]).

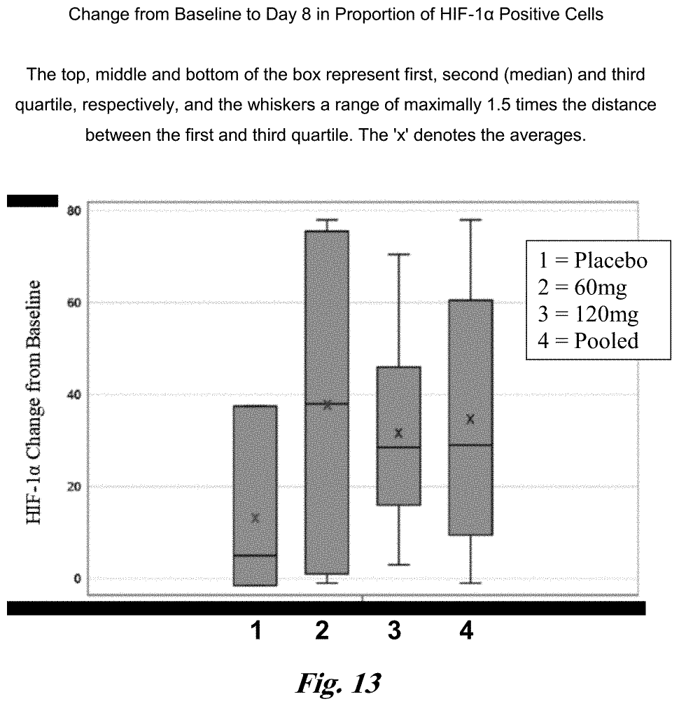

FIG. 13 shows the box plot of the proportions of HIF-1α positive cells evaluated at baseline and Day 8.

TABLE 50
Descriptive Statistic for Manually Assessed Proportions of HIF-1α Positive Cells
in HIF-1 α Stained Sections

| | | Placebo | 60 mg | 120 mg | Pooled | Total |
|---|---|---|---|---|---|---|
| Baseline | N | 3 | 6 | 6 | 12 | 15 |
| | Mean | 19.0 | 6.3 | 12.0 | 9.1 | 11.1 |
| | Std.Dev | 22.2 | 3.9 | 13.7 | 10.1 | 12.9 |
| | Median | 11.5 | 5.8 | 6.3 | 5.8 | 6.5 |
| | Min, Max | (1.5, 44.0) | (1.0, 11.0) | (2.5, 38.0) | (1.0, 38.0) | (1.0, 44.0) |
| Day 8 | N | 4 | 6 | 6 | 12 | 16 |
| | Mean | 44.5 | 44.5 | 44.1 | 44.3 | 44.3 |
| | Std.Dev | 28.9 | 32.6 | 24.2 | 27.4 | 26.8 |
| | Median | 44.0 | 46.5 | 45.3 | 45.3 | 44.8 |
| | Min, Max | (10.0, 80.0) | (4.0, 79.0) | (5.5, 79.0) | (4.0, 79.0) | (4.0, 80.0) |
| CFB | N | 3 | 6 | 6 | 12 | 15 |
| | Mean | 13.7 | 38.3 | 32.1 | 35.2 | 30.9 |
| | Std.Dev | 20.9 | 35.2 | 23.8 | 28.8 | 28.2 |
| | Median | 5.0 | 38.0 | 28.5 | 29.0 | 25.5 |
| | Min, Max | (−1.5, 37.5) | (−1.0, 78.0) | (3.0, 70.5) | (−1.0, 78.0) | (−1.5, 78.0) |

CFB: change from baseline; CI: confidence interval; HIF-1α: hypoxia inducible factor 1 alpha; Min: minimum; Max: maximum; N: sample size; Std Dev: standard deviation

Serum samples were collected for PD analysis and revealed no Compound 1-related effects on circulating EPO and VEGF levels.

### Example 25: A Phase 1b, Randomized, Double-Blind, Placebo-Controlled, Clinical Study to Evaluate the Safety, Tolerability, Pharmacokinetics and Pharmacodynamics of Compound 1 (Solution) in Adult Patients with Active Ulcerative Colitis

Compound 1 is administered as the HCl-salt, thus:

“120 mg dose” refers to 130 mg Compound 1-HCl salt—prepared analogously to Example 2.1 (equivalent to 120 mg free Compound 1.)
“60 mg dose” refers to 65 mg Compound 1-HCl salt—prepared analogously to Example 2.1 (equivalent to 60 mg free Compound 1.)

The objectives and endpoints of the study are as follows:
Primary Objective: Evaluate the safety and tolerability of Compound 1 daily dosing for 28 days
Secondary Objective: Evaluate the pharmacokinetics (PK) of Compound 1
Exploratory Objective: Explore target engagement and pharmacodynamic response; clinical and histologic activity; and evaluate the relationship between safety, disease activity, exposure parameters and pharmacogenetics
Primary Endpoints: Incidence of treatment-emergent adverse events (TEAEs) and changes from baseline in laboratory, vital sign and electrocardiogram parameters
Secondary Endpoint: PK parameters
Exploratory Endpoint: Changes in biomarkers of target engagement, pharmacodynamics, clinical activity scores, UC-100 Mayo Clinic Score and Robarts Histopathology Index; safety, disease activity and exposure parameters; and proportion of patients with histologic remission and/or mucosal healing at Day 28

In clinical trials, disease activity has traditionally been assessed by evaluating signs and symptoms of the disease, rather than the inflammatory process itself. Endoscopic appearance and histology have recently been validated as clinical trial outcome measures that provide for more direct assessment of disease activity and mucosal healing. Indeed, histology is an important prognostic factor and treatment target, providing insight into underlying histologic disease activity. The composite UC-100 score calculated as: (1+16× Mayo Clinic stool frequency subscore [0 to 3]+6× Mayo Clinic endoscopic subscore [0 to 3]+1× Robarts histopathology index score [0 to 33]), ranges from 1 (no disease activity) to 100 (severe disease activity). Validation of the composite UC-100 Index allows clinical trials the ability to reliably measure the combination of symptoms, endoscopic appearance and histologic activity.

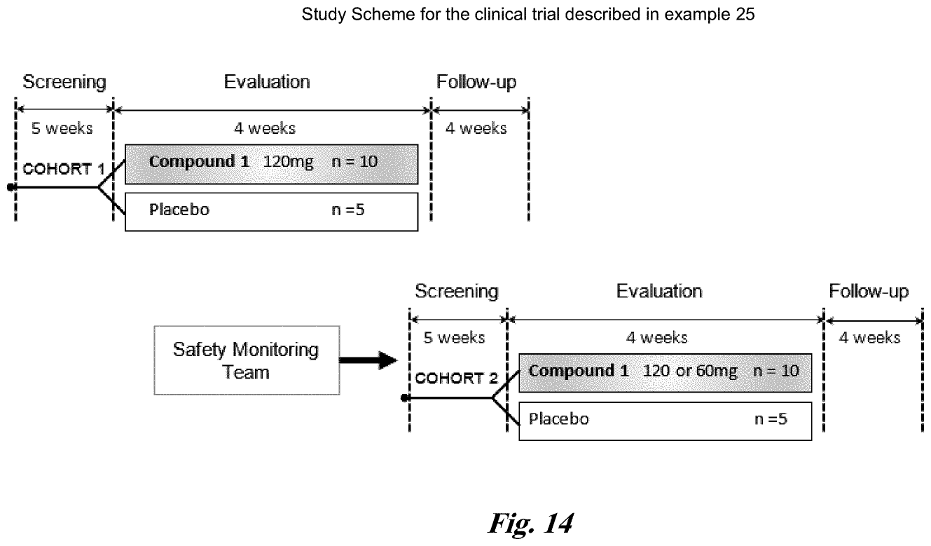

The study employs a two-cohort design, shown in schematic form in FIG. 14.

Cohort 1 evaluates Compound 1 120 mg, relative to placebo.
Cohort 2 studies additional patients at the 120 mg or 60 mg dose levels. A safety monitoring team determines whether and when to initiate Cohort 2 following review of Cohort 1 data, and if so, the 120 mg or 60 mg dose to be evaluated. The total number of patients is approximately 30. Each cohort (Cohort 1 and 2) randomizes approximately 15 patients, with approximately 10 patients randomized to Compound 1 and 5 patients to placebo.

Compound 1, or placebo, is administered one daily (QD), every day for 28 days, as an oral solution. Each dose is reconstituted with approximately 100 mL water, followed by an approximately 100 mL water “rinse” from the bottle contain Compound 1. Each dose also contains 10 g hydroxypropyl-beta-cyclodextrin (HPβCD). Placebo dosing solutions are identical to active, except without Compound 1.

The total duration of the study per patients is 13 weeks, including a 5-week screening period (7-35 days), the 4-week treatment period and a 4-week follow-up period.

Dose are administered orally in the morning at approximately the same time each day, with patients fasting for at least 6 hours before and 2 hours after each dose. The first dose is administered on Day 1. A blood sample is taken 8 hours post-dose. Patients are assessed on Days 7, 14, 21 and 28, followed by a Follow-up (FU) visit four weeks later.

Patients are age 18-75, male or female with a BMI 18-35 kg/m². Patients do not have Crohn's disease or indeterminate colitis, pouchitis, evidence of Clostridium difficile infection, a current malignancy or previous history of cancer or a history of alcohol or substance abuse. Patients exhibit the following disease criteria:
1. Positive ulcerative colitis diagnosis.
2. Complete Mayo clinic score of 3-12, with an endoscopic subscore 1.
3. The presence of blood in the stool.
4. Colonic biopsies with evidence of histologic activity defined by a Robarts Histopathology Index 4 with neutrophils in the epithelium (subscore 1).
5. Currently receiving treatment with oral 5-aminosalicylates (5-ASA) for ulcerative colitis or prednisone or equivalent, budesonide mmx or azathioprine or 6-mercaptopurine.
6. Evidence of UC extending 15 cm from the anal verge.

The efficacy endpoints are:
Complete Mayo Clinic Score: The sum of the Stool Frequency subscore, Rectal Bleeding subscore, Physician Global Assessment, and the Endoscopy subscore. Each subscore ranges from 0 to 3 points and the Complete Mayo score ranges from 0 to 12 points.
Improvement in Endoscopic Appearance: Mayo Endoscopy subscore of 0 or 1, or 0 if baseline Mayo Endoscopy subscore=1.
Mucosal Healing: Mayo Endoscopy subscore of 0 or 1, or 0 if baseline Mayo Endoscopy subscore=1, and Robarts Histopathology Index (RHI) Score≤3 with lamina propria neutrophils subscore=0 and neutrophils in epithelium subscore=0.
Histologic Remission: RHI s 3 with lamina propria neutrophils subscore=0 and neutrophils in epithelium subscore=0.
UC-100 Score: The composite index (1+16× Mayo Clinic Stool Frequency Subscore [0 to 3]+6× Mayo Clinic Endoscopic Subscore [0 to 3]+1× Robarts Histopathology Index Score [0 to 33]).
Robarts Histopathology Index [RHI] Score: Consists of four items: chronic inflammatory infiltrate, lamina propria neutrophils, neutrophils in epithelium, and erosion or ulceration. The RHI ranges from 0-33.

Safety is assessed by physical examination, vital signs (pulse, respiratory rate, temperature and blood pressure), an electrocardiogram (ECG) and laboratory assessments.

Blood samples (˜3 mL) are collected on the Day 1 and 28 visits, 0.5, 1, 2, 3, 4, 6, and 8, 12, and 24 hours post-dose. Stool samples, when available, are collected on Study Days 14 and 28.

Blood, stool and colonic biopsy samples evaluate the effect of Compound 1 on a range of potential target engagement and PD biomarkers.

Throughout the study, patients record stool frequency and rectal bleeding symptoms. The Rectal Bleeding and Stool Frequency components of the Mayo Score are calculated based on the most recent 3 days with data prior to the Visit, excluding the day of and the day after a flexible sigmoidoscopy/colonoscopy, and the day(s) of bowel preparation. These 3 days must occur within the 7 days prior to the Visit.

Flexible sigmoidoscopy evaluates the endoscopic appearance of the colonic mucosa. At Baseline and Day 28 (and Day 14 if applicable), 6 biopsies are collected from the rectum and sigmoid and evaluated for PK and histologic, immunohistochemical, and gene expression signals of biological activity.

Clinical Laboratory Assessments include hematology (white blood cell count, red blood cell, hemoglobin, hematocrit, platelet count); clinical chemistries (alanine aminotransferase, albumin, aspartate aminotransferase, alkaline phosphatase, bicarbonate, bilirubin, blood urea nitrogen, calcium, creatinine, chloride gamma-glutamyl transferase, glucose, L-lactate dehydrogenase, potassium, total protein, sodium; and urinalysis (dipstick, including macroscopic appearance, bilirubin, blood, color, glucose, ketones, leukocyte esterase, nitrite, pH, protein, specific gravity, urobilinogen).

### Example 26: A Phase 1a, Randomized, Double-Blind, Placebo-Controlled, Multiple Dose Study to Assess the Safety, Tolerability, Pharmacokinetics, and Pharmacodynamic Effects of Tablet Formulations of Compound 1 in Healthy Male and Female Volunteers

The primary objective of the study is to assess the safety and tolerability of tablet formulations of Compound 1⋅HCl after multiple oral doses. The safety endpoints include incidence of treatment-emergent adverse events (TEAEs) and changes in laboratory, vital sign and ECG parameters.

The secondary objective is to evaluate the pharmacodynamic response of tablet formulations after multiple oral doses, with an endpoint of change in of target engagement biomarkers (e.g., CAIX). Target engagement is assessed by measuring HIF-1 related gene expression and protein abundance in colonic tissue, stool and blood.

An exploratory objective of the study is to evaluate the effect of pharmacogenetics (PGx) on pharmacokinetic (PK) parameters of tablet formulations as compared to oral solution. Pharmacokinetics assessments include evaluation of blood, stool and colonic biopsy samples.

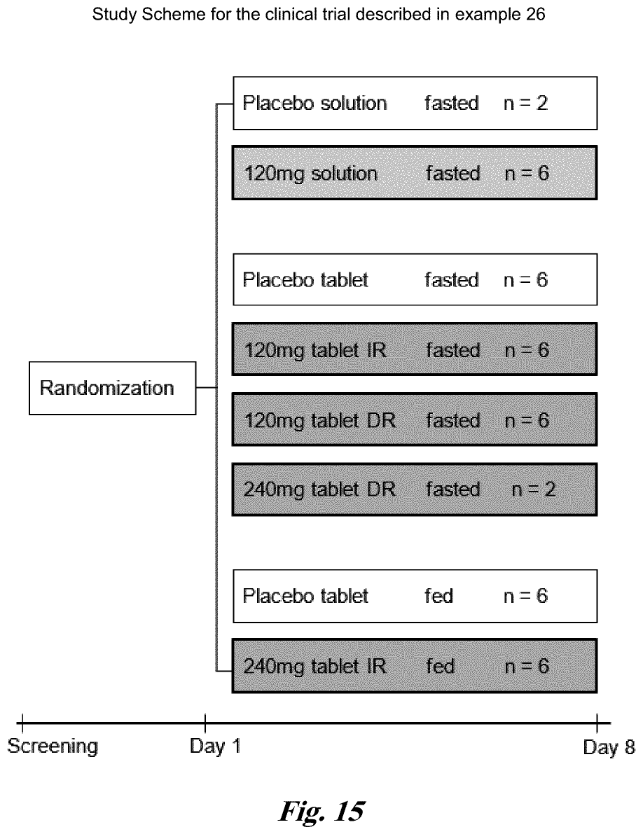

A schematic of the study design is presented in FIG. 15. 40 subjects are randomized in the study, stratified by gender with each stratum comprising half of the study population. Thirty subjects are randomized to Compound 1 and 10 to placebo. Up to 8 replacement subjects may be added (up to 2 in the solution groups and up to 6 in the tablet groups; each replacement is the same gender and assigned to the same treatment group as the subject being replaced). Subjects are randomized to 8 groups, according to Table 51.

TABLE 51
Study Interventions

| Cohort | 1 | 2 | 3 | 4 | 5 | 6 | 7 | 8 |
|---|---|---|---|---|---|---|---|---|
| Treatment | Placebo | Cmpd 1 | Placebo | Cmpd 1 | Cmpd 1 | Cmpd 1 | Placebo | Cmpd 1 |
| Dose | | 120 mg | | 120 mg | 120 mg | 240 mg | | 240 mg |
| Dosage | SoIn | SoIn | Tab | Tab IR | Tab DR | Tab DR | Tablets | Tab IR |
| Form | | | | | | | | |
| Fed/Fasted | Fasted | Fasted | Fasted | Fasted | Fasted | Fed | Fed | Fasted |
| n (planned) | 2 | 6 | 6 | 6 | 6 | 6 | 6 | 2 |

The study duration is up to 65 days, and includes the following periods:

Screening (2-28 days)

Treatment period of 8 days (treatments administered once daily for 7 days)

End of study visit, 10 days post-last dose [Day 17]

Follow-up phone call, 28 days post-last dose [Day 35]

Subjects are healthy males or females, age 18-65 with a Body Mass Index (BMI) 18-33 kg/m². Subjects were infection free (including HIV, hepatitis B or hepatitis C), no history of chronic disease or cancer, alcohol dependence, drug addiction or nicotine use, and did not display any significant colorectal symptoms or findings.

Subject are dosed orally in the morning, at the same time each day, once daily for 7 days. Doses are administered as follows:
Solution: Compound 1 is administered in 100 mL of a water 10% hydroxypropyl-beta-cyclodextrin (HPβCD) solution. Each solution dose is mixed with 100 mL purified water, followed by a 100 mL water “rinse” taken orally from the same bottle.
Tablet: Compound 1—IR tablets prepared according to Example 17; DR tablets prepared according to Example 18. 60 mg tablets IR, DR or matching placebo formulations are similar and indistinguishable in terms of appearance, odor and taste. Subjects randomized to receive 120 mg, are administered two 60 mg tablets; subjects randomized to receive 240 mg, are administered four 60 mg tablets.
Fasting subjects, fast for at least 10 hours before and 2 hours after each dose.
Fed subjects are dosed on a fed stomach 30 minutes after the start of a standardized meal. Subjects are required to consume the meal in 30 minutes or less.

Physical Examinations include vital signs (pulse rate, respiratory rate, temperature and blood pressure) and electrocardiogram (ECG).

A flexible sigmoidoscopy is performed prior to dosing and on Day 7 to evaluate the endoscopic appearance of the colonic mucosa and obtain biopsies. During the sigmoidoscopy, 6 biopsies are collected in each of the two segments (e.g., Rectum and Sigmoid) for approximately 12 biopsies. Biopsies assess PK, immunohistochemical and gene expression signals (e.g., CAIX) of biological activity

Blood samples are collected on Days 1 and 7, at pre-dose and 0.25, 0.5, 1, 2, 3, 4, 6, and 8, 12, and 24 hours post-dose.

Stool samples, when available, are collected on Days 5 and 7.

Results from Example 26 Clinical Trial

### Example 27: Pharmacokinetics of Formulations of Compound 1 in Healthy Subjects

IR and DR tablet dosing (120 mg or 240 mg dose) was compared to solution dosing in a 10% hydroxypropyl-beta-cyclodextrin (HPβCD) solution (120 mg dose) according to the protocol described in Example 26 above. This was a Phase 1a, randomized, double-blind, placebo-controlled, multiple dose study that evaluated the safety, tolerability, pharmacokinetics (PK), and pharmacodynamics of tablet formulations of Compound 1 in male and female healthy adult subjects, and compared PK parameters of Compound 1 tablet formulations to Compound 1 oral solution and fasted to fed subjects. The composition and method of manufacture of the IR and DR tablet compositions used in this study are disclosed in Examples 17 and 18 above.

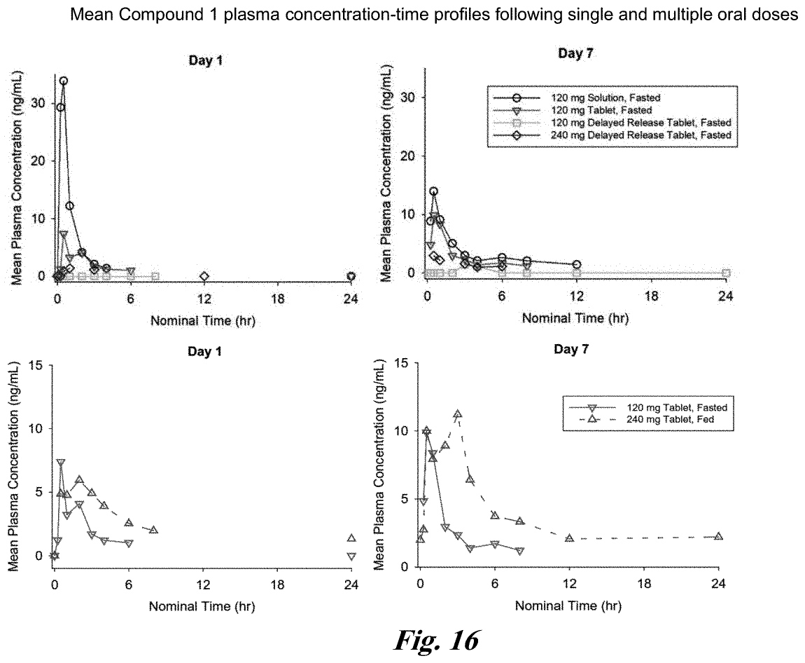

Mean plasma concentration-time profiles of Compound 1 on Day 1 and Day 7 are shown in FIG. 16.

On Day 1, Compound 1 was rapidly absorbed from all fasting treatments with maximum plasma concentrations occurring at a median Tₘₐₓ of 0.500 to 1.00 hours post-dose, but delayed to a median Tₘₐₓ of 2.00 hours following administration of the IR tablet at a dose of 240 mg, fed.

Tₘₐₓ values of the DR tablet were relatively longer than the other tablet treatment groups on Day 7. On Day 1, except for the 120 and 240 mg delayed release tablet formulations, all Compound 1 treated subjects displayed a quantifiable concentration by 0.5 hours post-dose. Five of 6 and 3 of 6 subjects receiving the 120 and 240 mg delayed release tablet formulations, respectively, displayed quantifiable concentrations below the lower limit of quantitation at all time points. There was considerable inter-subject variability in plasma concentrations up to 200% in all treatment groups.

On Day 7, all subjects had quantifiable concentrations throughout most of the sampling period except for two subjects in each of the delayed release tablet formulation groups.

Overall, mean Compound 1 concentrations on Day 7 were slightly higher than on Day 1 for all treatment groups, except for the 120 mg solution.

Summary statistics for Compound 1 PK parameters in plasma for Day 1 and Day 7 are presented in Tables 52 and 53, respectively.

TABLE 52
Arithmetic Mean and % CV Pharmacokinetic Parameters in Plasma for Compound 1 on Day 1

| | 120 mg | | | 120 mg | | | 120 mg | | | 240 mg | | | 240 mg | | |
|---|---|---|---|---|---|---|---|---|---|---|---|---|---|---|---|
| | Solution, | | | IR Tablet, | | | DR Tablet, | | | DR Tablet, | | | IR Tablet, | | |
| | Fasted | | | Fasted | | | Fasted^{a} | | | Fasted^{a} | | | Fed | | |
| | n | Mean | % CV | n | Mean | % CV | n | Mean | % CV | n | Mean | % CV | n | Mean | % CV |
| T_{max (h)} | 7 | 0.500 | | 6 | 0.500 | | 1 | NC^{c} | | 3 | 1.00 | | 7 | 2.00 | |
| | | (0.250, 1.00)^{b} | | | (0.250, 4.00)^{b} | | | | | | (0.500, 2.00)^{b} | | | (0.500, 4.00)^{b} | |
| Cₘₐₓ | 7 | 34.5 | 179 | 6 | 8.85 | 76.8 | 6 | 0.428 | 245 | 6 | 1.80 | 135 | 7 | 9.20 | 61.6 |
| (ng/mL) | | | | | | | | | | | | | | | |
| AUCₗₐₛₜ | 7 | 39.3 | 115 | 6 | 16.9 | 75.7 | 6 | 9.71 | 245 | 6 | 10.9 | 163 | 7 | 48.9 | 66.0 |
| (ng * h/mL) | | | | | | | | | | | | | | | |
| AUC_{inf} | 6 | 45.6 | 105 | 1 | NC^{c} | NC^{c} | 0 | NC | NC | 0 | NC | NC | 6 | 64.3 | 68.8 |
| (ng * h/mL) | | | | | | | | | | | | | | | |
| Half-life(h) | 6 | 1.13 | 44.3 | 1 | NC^{c} | NC^{c} | 0 | NC | NC | 0 | NC | NC | 6 | 4.20 | 54.5 |

Abbreviations: AUC_{inf}, area under the concentration-time curve from zero to infinity; AUCₗₐₛₜ, area under the concentration-time curve from zero to the last quantifiable concentration time point; Cₘₐₓ, maximum plasma drug concentration; CV, coefficient of variation; NC, not calculated; Tₘₐₓ, time to reach Cₘₐₓ.
^{a}Includes subjects who had Compound 1 concentrations below the limit of quantitation at all time points (5 subjects in the 120 mg Delayed Release Tablet and 3 in the 240 mg Delayed Release Tablet treatment groups); for these subjects, Cₘₐₓ and AUCₗₐₛₜ were set = 0.00 and Tₘₐₓ was not calculated.
^{b}Median (range)
^{c}PK parameter could only be determined in one subject and thus, not included in summary statistics.

TABLE 53
Arithmetic Mean and % CV Pharmacokinetic Parameters in Plasma for Compound 1 on Day 7

| | 120 mg | | | 120 mg | | | 120 mg | | | 240 mg | | | 240 mg | | |
|---|---|---|---|---|---|---|---|---|---|---|---|---|---|---|---|
| | Solution, | | | IR Tablet, | | | DR Tablet, | | | DR Tablet, | | | IR Tablet, | | |
| | Fasted | | | Fasted | | | Fasted^{a} | | | Fasted^{a} | | | Fed | | |
| | n | Mean | % CV | n | Mean | % CV | n | Mean | % CV | n | Mean | % CV | n | Mean | % CV |
| Tₘₐₓ (h) | 7 | 0.500 | | 6 | 0.500 | | 4 | 3.00 | | 4 | 3.50 | | 6 | 0.500 | |
| | | (0.250, 1.00)^{b} | | | (0.250, 0.500)^{b} | | | (2.00, 4.00)^{b} | | | (0.500, 6.00)^{b} | | | (0.500, 3.00)^{b} | |
| Cₘₐₓ (ng/mL) | 7 | 14.4 | 17.1 | 6 | 10.6 | 86.8 | 6 | 2.11 | 141 | 6 | 3.62 | 129 | 6 | 18.4 | 73.9 |
| AUCₗₐₛₜ | 7 | 42.4 | 40.6 | 6 | 31.5 | 72.7 | 6 | 15.2 | 157 | 6 | 16.9 | 125 | 6 | 99.9 | 73.8 |
| (ng * h/mL) | | | | | | | | | | | | | | | |
| Cₘₐₓ/AUCₗₐₛₜ | | 0.34 | | | 0.34 | | | 0.14 | | | 0.21 | | | 0.18 | |
| Half-life(h) | 7 | 2.45 | 124 | 5 | 3.54 | 93.8 | 1 | NC^{c} | NC^{c} | 2 | NC^{c} | NC^{c} | 6 | 2.67 | 31.9 |
| AR_{Cmax} | 7 | 1.26 | 62.7 | 6 | 1.54 | 92.1 | 1 | NC^{c} | NC^{c} | 2 | NC^{c} | NC^{c} | 6 | 1.81 | 34.7 |
| AR_{AUC0-24} | 7 | 1.57 | 43.3 | 6 | 3.31 | 103 | 1 | NC^{c} | NC^{c} | 2 | NC^{c} | NC^{c} | 6 | 1.99 | 57.9 |

Abbreviations: AR_{AUC0-24} = accumulation rato of AUC from time 0 to 24 hours; ARCₘₐₓ = accumulation ratio of Cₘₐₓ; AUCₗₐₛₜ = area under the concentration-time curve from zero to the last quantifiable concentration time point; Cₘₐₓ = maximum plasma drug concentration; CV = coefficient of variation; NC = not calculated; Tₘₐₓ = time to reach Cₘₐₓ.
^{a}Includes subjects who had Compound 1 concentrations below the limit of quantitation at all time points (2 in the 120 mg Delayed Release Tablet and 2 in the 240 mg DR) mg) Delayed Release Tablet treatment groups; for these subjects, Cₘₐₓ and AUCₗₐₛₜ were set = 0.00 and Tₘₐₓ was not calculated.
^{b}Median (range)
^{c}Could be determined in less than 3 subjects and thus, not included in summary statistics.

On Day 1, half-life (t_{1/2}) and exposure (AUC_{inf}) could not be estimated accurately due to the lack of evaluable data points available from most subjects except for those receiving the 120 mg solution fasting and 240 mg tablet fed treatments. The derived values for these parameters should be interpreted with caution.

Mean Cₘₐₓ and AUCₗₐₛₜ for the fasted 120 mg solution were slightly higher than the fasted 120 mg IR tablet on Day 1 (34.5 ng/mL and 39.3 ng*h/mL vs. 8.85 ng/mL and 16.9 ng*h/mL, respectively), but comparable on Day 7 (14.4 ng/mL and 42.4 ng*h/mL and 10.6 ng/mL and 31.5 ng*h/mL). On Day 1, as previously stated, 5 of 6 and 3 of 6 subjects receiving the 120 and 240 mg delayed release tablet formulations had no detectable levels of Compound 1 in plasma and most PK parameters could not be accurately determined. The same was true on Day 7, where two of six subjects receiving the 120 and 240 delayed release tablet formulations had no detectable levels of Compound 1. Except for a slight delay in Tₘₐₓ following administration of food with the 240 mg tablet on Day 1, the Cₘₐₓ and AUCₗₐₛₜ, following dose normalization, were similar to that following administration of 120 mg tablet fasting.

Exposure of the solution was slightly higher than the tablet following a single dose but were comparable on Day 7. Following the first dose on Day 1, Compound 1 was rapidly absorbed from all fasting treatments with median Tₘₐₓ of 0.500-1.00 h but delayed to 2.00 h after a meal. However, upon once daily dosing for 7 days, Compound 1 was rapidly absorbed with a median Tₘₐₓ of 0.500 h for all treatments except for the 120 mg and 240 mg delayed release tablet formulations where the median Tₘₐₓ values were 3.00 and 3.50 h, respectively. The majority of subjects receiving the delayed release tablets had no detectable levels of Compound 1 at any time point on Day 1; however, upon multiple dosing, more subjects had plasma concentrations above the lower limit of quantitation on Day 7.

### Example 28: Colonic Tissue Concentrations of Tablet Formulations of Compound 1 in Healthy Subjects

IR and DR tablet dosing (120 mg or 240 mg dose) was compared to solution dosing in terms of colon tissue (sigmoid and rectum) concentrations of Compound 1 according to the protocol described in Example 26 above.

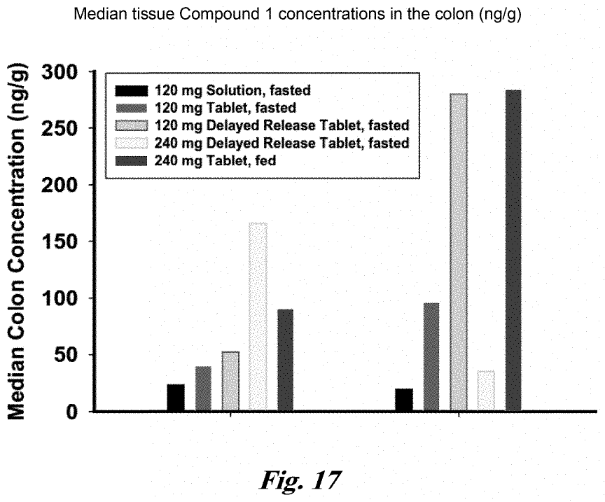

A summary of mean and median tissue Compound 1 concentrations in the colon on Day 7 is provided in Table 54 and FIG. 17.

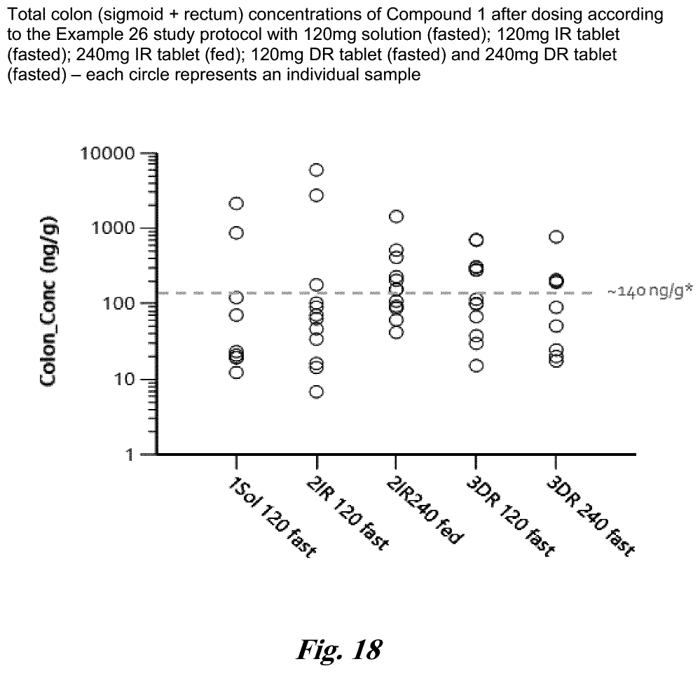

FIG. 18 shows the total drug colonic concentrations across both sections (sigmoid+rectum) for the different study subject cohorts.

TABLE 54
Tissue concentrations of Compound 1 in the Colon (ng/g), Day 7

| | Rectum | | | | | Sigmoid | | | |
|---|---|---|---|---|---|---|---|---|---|
| Formulation | n | Mean | % CV | Median (Range) | | Mean | % CV | Median (Range) | |
| 120 mg | 7 | 420 | 189 | 23.2 | (0-2130) | 151 | 211 | 19.3 | (0-868) |
| Solution | | | | | | | | | |
| Fasted | | | | | | | | | |
| 120 mg IR | 6 | 484 | 227 | 40.2 | (14.3-2730) | 1060 | 224 | 96.2 | (6.83-5920) |
| Tablet Fasted | | | | | | | | | |
| 120 mg DR | 6 | 90.1 | 123 | 52.5 | (0-306) | 298 | 73.5 | 280 | (100-706) |
| Tablet Fasted | | | | | | | | | |
| 240 mg DR | 6 | 127 | 72.7 | 166 | (0-207) | 161 | 187 | 35.3 | (16.7-770) |
| Tablet Fasted | | | | | | | | | |
| 240 mg IR | 6 | 119 | 65.4 | 90.1 | (41.6-229) | 463 | 108 | 284 | (108-1430) |
| Tablet Fed | | | | | | | | | |

Abbreviation: CV = coefficient of variation.

The median Compound 1 concentrations in the rectum were higher for the delayed release tablets as compared to the immediate release tablet and solution. However, while the median concentration of Compound 1 in the sigmoid was also higher for the 120 mg delayed release tablet than for the immediate release tablet and solution, it was less for the 240 mg delayed release tablet than for the immediate release tablet; however, inter-subject variability was notably high across all formulations (CV % ranged from 65.4% to 227%).

Despite distinct systemic PK profiles, the immediate release and delayed release tablets delivered similar median levels of Compound 1 to the colon and more than the solution dose.

### Example 29: Safety of Formulations of Compound 1 in Healthy Subjects

IR and DR tablet dosing (120 mg or 240 mg dose) was compared to solution dosing in terms of adverse events reported according to the study protocol described in Example 26 above.

Preliminary safety results in this study suggest that Compound 1 formulations up to 120 mg solution, 240 mg tablet, and 240 mg DR tablet are generally well tolerated in healthy subjects (Table 55). Furthermore, no consistent or dose-related effects of Compound 1 on EPO or VEGF from baseline to Day 7 were observed when compared to placebo.

TABLE 55
Adverse Events for Example 26 study cohorts

| | Cohort | | | | | | | |
|---|---|---|---|---|---|---|---|---|
| | | 120 mg | 240 mg | 120 mg | 240 mg | | | |
| | 120 mg | IR | IR | DR | DR | Placebo | Placebo | Placebo |
| | soln - | tablet - | tablet - | tablet - | tablet - | soln - | tablet - | tablet - |
| | fasted | fasted | fed | fasted | fasted | fasted | fed | fasted |
| n | 7 | 6 | 7 | 6 | 6 | 2 | 2 | 6 |
| Headache | 1 (14%) | 0 | 1 (14%) | 0 | 1 (17%) | 0 | 1 (50%) | 0 |
| Nausea | 0 | 1 (17%) | 0 | 1 (17%) | 1 (17%) | 0 | 0 | 0 |
| Diarrhoea | 1 (14%) | 1 (17%) | 0 | 0 | 0 | 1 (50%) | 0 | 1 (17%) |
| Dizziness | 0 | 1 (17%) | 0 | 0 | 0 | 0 | 0 | 0 |
| Liver | 0 | 0 | 1 (14%) | 0 | 0 | 0 | 0 | 0 |
| function | | | | | | | | |
| abnormal | | | | | | | | |
| Liver | 1 (14%) | 0 | 0 | 0 | 0 | 0 | 0 | 0 |
| function | | | | | | | | |
| increased | | | | | | | | |
| Rash | 0 | 1 (17%) | 0 | 0 | 0 | 0 | 0 | 0 |
| Upper resp. | 0 | 1 (17%) | 0 | 0 | 0 | 0 | 0 | 0 |
| tract | | | | | | | | |
| infection | | | | | | | | |
| Viral | 0 | 0 | 0 | 1 (17%) | 0 | 0 | 0 | 0 |
| infection | | | | | | | | |
| Vomiting | 0 | 0 | 0 | 0 | 1 (17%) | 0 | 0 | 0 |
| Number (%) | 2 (29%) | 3 (50%) | 2 (29%) | 1 (17%) | 1 (17%) | 1 (50%) | 1 (50%) | 1 (17%) |
| with AE | | | | | | | | |

1. An immediate release solid pharmaceutical composition for oral administration comprising a granulate, wherein the granulate comprises a pharmaceutically acceptable salt of Compound 1: 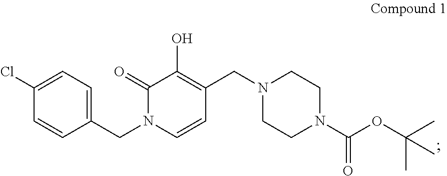 [SMILES: CC(C)(C)OC(=O)N1CCN(Cc2ccn(Cc3ccc(Cl)cc3)c(=O)c2O)CC1]
and one or more pharmaceutically acceptable excipients.

2. The composition according to claim 1, wherein the pharmaceutically acceptable salt is a hydrochloride salt.

3. The composition according to claim 2, wherein the ratio of Compound 1 to HCl is about 1:1.

4. The composition according to claim 2, wherein the hydrochloride salt is a monohydrate.

5. The composition according to claim 2, wherein the hydrochloride salt is anhydrous.

6. The composition according to claim 1, wherein the granulate comprises a mixture of the hydrochloride salt as a crystalline monohydrate and the hydrochloride salt as an amorphous compound.

7. The composition according claim 1, wherein the granulate comprises a mixture of the hydrochloride salt as a crystalline monohydrate and the hydrochloride salt as a crystalline anhydrous compound.

8-24. (canceled)

25. The composition according to claim 1, comprising 10-30% w/w of the pharmaceutically acceptable salt of Compound 1.

26. The composition according to claim 1, comprising 20 to 150 mg of the pharmaceutically acceptable salt of Compound 1.

27. The composition according to claim 1, wherein the one or more pharmaceutically acceptable excipients do not comprise a buffering agent.

28-30. (canceled)

31. The composition according to claim 1, wherein the composition undergoes substantially complete disintegration and dispersal in pH 6.8 aqueous media in less than 4 minutes at 37° C. using USP2 disintegration apparatus.

32. The composition according to claim 1, wherein the composition provides a geometric mean maximum plasma concentration (Cₘₐₓ) of Compound 1 after oral dosing of at least 5 ng/ml.

33. The composition according to claim 1, wherein the composition provides colonic tissue exposure greater than or equal to the systemic exposure of Compound 1.

34. (canceled)

35. The composition according to claim 1, formulated as a tablet.

36. (canceled)

37. The composition according to claim 35, wherein the tablet comprises a granulate component and an extra-granulate component.

38. The composition according to claim 37, wherein the granulate component comprises a pharmaceutically acceptable salt of Compound 1 and one or more pharmaceutically acceptable excipients selected from a filler, a diluent, a binder and a disintegrant.

39. The composition according to claim 37, wherein the extra-granulate component comprises one or more pharmaceutically acceptable excipients selected from a filler, a diluent, a disintegrant and a lubricant.

40. The composition according to claim 35, wherein the tablet is substantially encapsulated in a UV-resistant coating.

41. (canceled)

42. (canceled)

43. A process for forming an immediate release solid pharmaceutical composition according to claim 1, the process comprising the steps of
a. mixing a pharmaceutically acceptable salt of Compound 1 with one or more pharmaceutically acceptable excipients selected from a filler, a diluent, a binder and a disintegrant;
b. adding to the mixture from step a) a wetting agent, water and optionally a binder;
c. subjecting the mixture from step b) to wet granulation;
d. drying the wet granules from step c);
e. milling the dried granules to a particles size diameter of <1 mm;
f. optionally blending the milled granules from step e) with one or more pharmaceutically acceptable excipients selected from a filler, a diluent, a disintegrant and a lubricant;
g. optionally compressing the blended granule mixture from step f) into a tablet; and
h. optionally substantially encapsulating the granules from step f) or the tablet from step g) in a UV-resistant coating.

44-59. (canceled)

60. A delayed-release solid pharmaceutical composition for oral administration comprising Compound 1 or a pharmaceutically acceptable salt thereof: 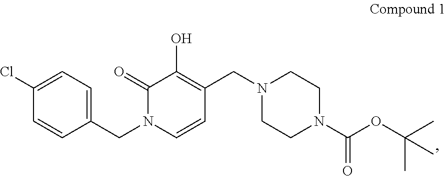 [SMILES: CC(C)(C)OC(=O)N1CCN(Cc2ccn(Cc3ccc(Cl)cc3)c(=O)c2O)CC1]
and one or more pharmaceutically acceptable excipients.

61. The composition according to claim 60, wherein the composition comprises a pharmaceutically acceptable salt of Compound 1.

62. The composition according to claim 61, wherein the pharmaceutically acceptable salt is a hydrochloride salt.

63. The composition according to claim 62, wherein the ratio of Compound 1 to HCl is about 1:1.

64. The composition according to claim 62, wherein the hydrochloride salt is a monohydrate.

65. The composition according to claim 62, wherein the hydrochloride salt is anhydrous.

66. The composition according to claim 60, wherein the composition comprises a core and a delayed release coating substantially encapsulating the core.

67. The composition according to claim 66, wherein the delayed release coating dissolves at pH values greater than about 5.5.

68-70. (canceled)

71. The composition according to claim 66, wherein the composition further comprises an additional sub-coating beneath the delayed release coating.

72. The composition according to claim 66, wherein the core comprises a granulate.

73-84. (canceled)

85. The composition according to claim 60, comprising 10-30% w/w of Compound 1 or a pharmaceutically acceptable salt thereof.

86. The composition according to claim 60, comprising 20 to 150 mg of Compound 1 or a pharmaceutically acceptable salt thereof.

87. The composition according to claim 60, wherein the one or more pharmaceutically acceptable excipients do not comprise a buffering agent.

88. The composition according to claim 60, wherein the composition undergoes less than 5% dissolution in 0.01N HCl after 30 mins at 37° C. using USP2 apparatus.

89. The composition according to claim 60, wherein the composition provides colonic tissue exposure greater than or equal to the systemic exposure of Compound 1.

90. The composition according to claim 60, formulated as a tablet.

91. (canceled)

92. The composition according to claim 90, wherein the tablet core comprises a granulate component and an extra-granulate component.

93. The composition according to claim 92, wherein the granulate component comprises Compound 1, or a pharmaceutically acceptable salt thereof, and one or more pharmaceutically acceptable excipients selected from a filler, a diluent, a binder and a disintegrant.

94. The composition according to claim 92, wherein the extra-granulate component comprises one or more pharmaceutically acceptable excipients selected from a filler, a diluent, a disintegrant and a lubricant.

95-97. (canceled)

98. The composition according to claim 60, wherein the composition undergoes substantially complete disintegration and dispersal in pH 6.8 aqueous media in less than 30 minutes at 37° C. using USP2 apparatus.

99. A process for forming a delayed-release solid pharmaceutical composition according to claim 72, the process comprising the steps
a) mixing Compound 1, or a pharmaceutically acceptable salt thereof, with one or more pharmaceutically acceptable excipients selected from a filler, a diluent, a binder and a disintegrant;
b) adding to the mixture from step a) a wetting agent, water and optionally a binder;
c) subjecting the mixture from step b) to wet granulation;
d) drying the wet granules from step c);
e) milling the dried granules to a particle size diameter of <1 mm;
f) optionally blending the milled granules from step e) with one or more pharmaceutically acceptable excipients selected from a filler, a diluent, a disintegrant and a lubricant; and either:
g1) compressing the blended granule mixture from step e) or step f) into a tablet;
h) optionally substantially encapsulating the tablet from step g) in a coating; and
i) substantially encapsulating the composition from step h) in a delayed release coating; or
g2) substantially encapsulating the granules from step e) or step f) in a delayed release coating or a delayed release capsule.

100-103. (canceled)

104. A method of treating diseases or conditions mediated alone, or in part, by PHD, the method comprising administering to a subject a therapeutically effective amount of an immediate release solid pharmaceutical composition according to claim 1.

105. The method according to claim 104, wherein the disease or condition is an inflammatory bowel disease.

106-111. (canceled)

112. A method of treating diseases or conditions mediated alone, or in part, by PHD, the method comprising administering to a subject a therapeutically effective amount of a delayed-release solid pharmaceutical composition according to claim 60.

113. The method according to claim 112, wherein the disease or condition is an inflammatory bowel disease.
